(19) United States
(12) Patent Application Publication
(10) Pub. No.: US 2024/0402159 A1
(43) Pub. Date: Dec. 5, 2024

(54) METHOD FOR DETECTING ANALYTES WITH METAL-ORGANIC FRAMEWORKS

(71) Applicant: UNIVERSITY OF UTAH RESEARCH FOUNDATION, Salt Lake City, UT (US)

(72) Inventors: Ling Zang, Salt Lake City, UT (US); Rana Dalapati, Salt Lake City, UT (US)

(21) Appl. No.: 18/680,856

(22) Filed: May 31, 2024

Related U.S. Application Data
(60) Provisional application No. 63/470,564, filed on Jun. 2, 2023.

(51) Int. Cl. G01N 33/53; G01N 33/543

(52) U.S. Cl. CPC G01N 33/5308; G01N 33/54326

(57) ABSTRACT

Described are PDI-based and UiO-66-based cationic metal-organic frameworks methods for detecting perfluoroalkyl and polyfluoroalkyl substances.

### CROSS-REFERENCE TO RELATED APPLICATION(S)

This application claims priority to U.S. Provisional Patent Application No. 63/470,564, filed on Jun. 2, 2023, the entire contents of which are fully incorporated herein by reference.

### TECHNICAL FIELD

The present disclosure describes PDI-based and UiO-66-based metal-organic frameworks (MOFs) for the detection of perfluoroalkyl and polyfluoroalkyl substances.

### BACKGROUND

Perfluoroalkyl and polyfluoroalkyl substances (PFAS) are a large group of laboratory-made fluorinated hydrocarbons (FHC) used to manufacture hydrophobic polymer coatings and products. Fluoropolymer coatings using PFAS are present in a variety of products, including non-stick cooking surfaces, furniture, aqueous film forming foam (AFFF) for fire extinguishing, adhesives, food packaging, clothing, and the insulation of electrical wire. The most used PFAS are perfluorooctanoic acid (PFOA) and perfluorooctane sulfonic acid (PFOS). During industrial production and widespread use of PFAS materials, it can easily seep into the soil, sludge, ground water, and air. Several PFAS, including PFOA and PFOS, do not break down in normal environmental conditions.

Due to the non-degradable nature of PFAS, dangerous and high levels can accumulate over time. Early epidemiological studies have identified links between PFOA exposure and high cholesterol, pregnancy-induced hypertension, thyroid illness, kidney and testicular cancer. The research has also disclosed that PFAS may affect the growth and learning behavior of newborns, reduce vaccine-induced immunity in children, affect the body's natural hormone production and immune system, and increase the risk of some cancers. Due to the high stability and persistence in the environment, PFAS compounds have a predisposition to bioaccumulate. The uptake of PFAS into plants and fish can occur within contaminated areas, and subsequently move into the food chain, potentially affecting wildlife and humans who consume these organisms. Thus, toxic PFAS compounds are bioaccumulated and then biomagnified up the food chain. Additionally, PFAS accumulation has been found in the blood of people and animals in various countries all over the world, which is a concern considering potential adverse health effects of PFAS. Therefore, the identification of toxic PFAS in contaminated water and removal of PFAS is becomes increasingly essential.

Currently, the standard detection techniques for PFAS primarily rely on chromatography coupled with mass spectrometry. The EPA approved official analytical techniques (“Method 537” and “Method 533”) to study different PFAS concentrations in drinking water. There is also the method of solid-phase extraction (SPE)-enabled liquid chromatography-tandem mass spectrometry (LC-MS/MS). Although the LC-MS method is accurate, it requires highly-trained laboratory personnel, complex instrumentation, and tedious sample preparation and data collection. In recent years, numerous efforts have been attempted to design a new PFAS detection platform that is not mass spectrometry dependent, is cost effective, has a fast response time, and is easy to use for onsite PFAS detection. Optical sensor materials have some intrinsic advantages, like fast response time, easy operation, cost effectiveness, naked-eye identification, and the potential to build hand-held sensor devices for onsite detection. There have been several fluorescent sensors recently developed to detect PFAS. Unfortunately, most of these reported fluorescence-based sensors currently lack sufficient selectivity, making it hard to distinguish among multiple PFAS. Considering the real field applications wherein mixture of PFASs may exist, it is essential to develop an easy-to-use, cost-effective fluorescence detection platform that can discriminatively detect against different PFAS. Selective detection of PFAS is also critical for assessing the transport of different types of PFAS in the environment and monitoring the effectiveness of abatement processes onsite.

Fluorescent metal-organic frameworks (MOFs) have evolved as a next-generation functional material because of their ultra-high porosity, large surface area, structural diversity and tunability. Based on these unique properties, a large number of MOF-based sensors have been developed in recent years for a broad range of applications in the field of fluorescence sensing. These applications include detection of volatile organic compounds, explosives, biomolecules, heavy metals, and many more. Very recently, three known zirconium porphyrinic MOFs (PCNs, i.e., PCN-222, PCN-223, and PCN-224) have been explored for PFAS detection using sensor arrays. The method used by others relies on a fluorescence “turn-off” response to detect PFAS. However, this approach can result in false “turn-off” signals due to unwanted contamination. In contrast, using fluorescent “turn-on” probes that switch their fluorescence from “off” (fluorescence quenching) to “on” (or fluorescence activating) not only amplifies the target signals but also reduces the background signal, resulting in a lower detection limit and improved sensitivity.

Therefore, what is needed are novel fluorescent MOF-based fluorescence activating probes and corresponding methods for the selective detection and removal of PFAS with said MOF-based fluorescence activating probes.

### SUMMARY

One embodiment described herein is a composition comprising at least one metal-organic framework (MOF), or salt thereof, the metal-organic framework comprising: a plurality of metal-containing secondary building units (SBU) linked together by one or more bridging ligands, wherein said bridging ligands, or salts thereof, have a formula (I):

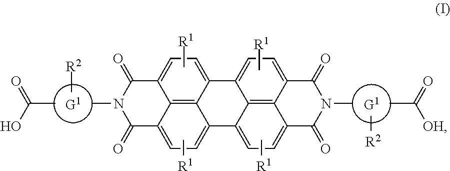

wherein
R¹, at each occurrence, is independently hydrogen, C₁₋₈alkyl, halo, —O(R^{1a}), —N(R^{x})ₘ, or —S(O)₂OH;
R², at each occurrence, is independently hydrogen, C₁₋₈alkyl, —O(R^{1a}), or C₁₋₈alkyl-OH and wherein every R² is not hydrogen;
G¹, at each occurrence, is independently phenyl, a 6- to 12-membered aryl, or a 5- to 12-membered heteroaryl, wherein the 5- to 12-membered heteroaryl is optionally substituted at a heteroatom with hydrogen or C₁₋₈alkyl;
R^{1a}, at each occurrence, is independently hydrogen or C₁₋₈alkyl, or phenyl;
R^{x}, at each occurrence, is independently hydrogen or C₁₋₃alkyl; and
m=2-3.
   In one aspect, G¹, at each occurrence, is phenyl. In another aspect, the bridging ligands, or salts thereof, have a formula (Ia):

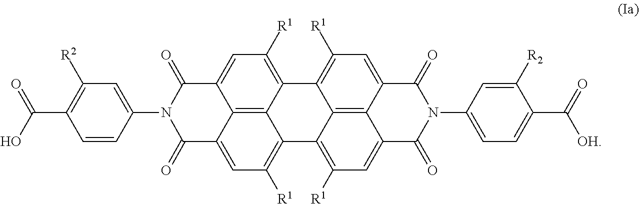

In another aspect, R¹, at each occurrence, is halo. In another aspect, R¹, at each occurrence, is chloro. In another aspect, R², at each occurrence, is C₁₋₈alkyl. In another aspect, R², at each occurrence is C₁alkyl. In another aspect, the bridging ligands, or salts thereof, are selected from:

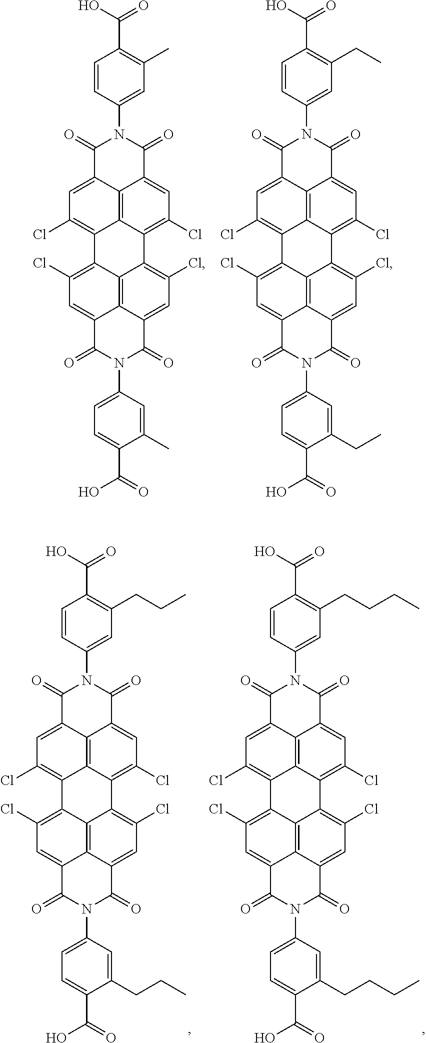

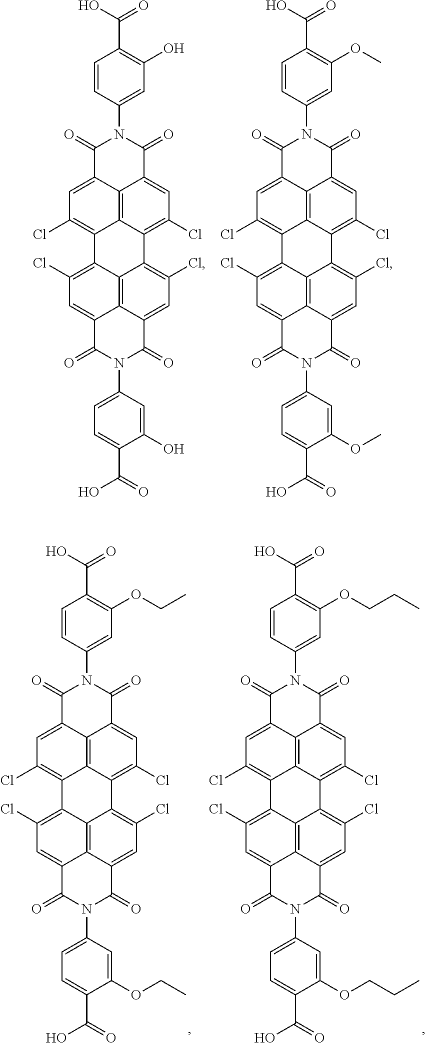

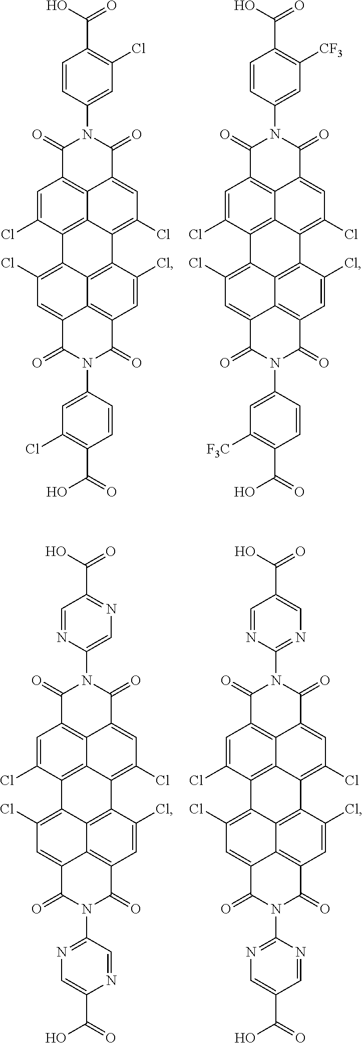

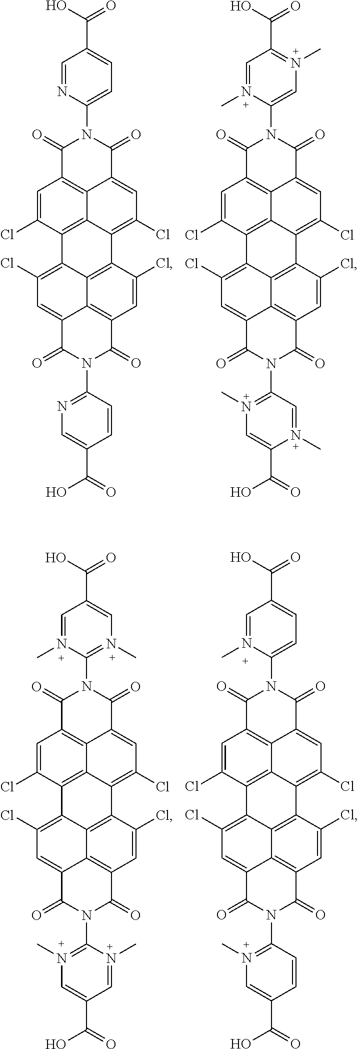

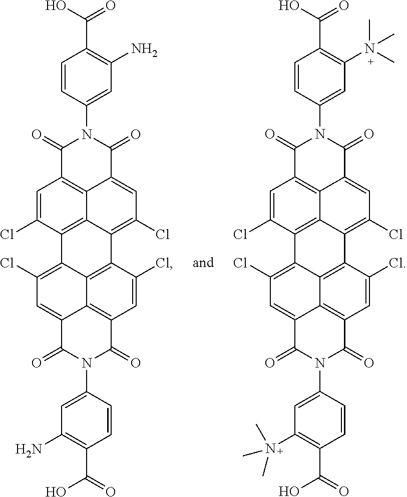

In another aspect, the secondary building units comprise one or more metal atoms and one or more carboxylates. In another aspect, the composition further comprises one or more modulators. In another aspect, the modulators are trifluoroacetic acid. In another aspect, the metal atoms comprise zirconium(IV) or hafnium(IV).

Another embodiment described herein is a method for detecting or removing perfluoroalkyl or polyfluoroalkyl substances in a sample, the method comprising: contacting the sample with a composition comprising at least one metal-organic framework (MOF), or salt thereof, having a plurality of metal-containing secondary building units (SBU) linked together by one or more bridging ligands, wherein said bridging ligands, or salts thereof, have a formula (I):

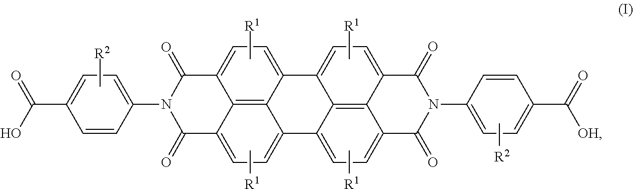

wherein
R¹, at each occurrence, is independently hydrogen, C₁₋₈alkyl, or halo;
R², at each occurrence, is independently hydrogen, C₁₋₈alkyl, —O(R^{1a}), or C₁₋₈alkyl-OH; and
R^{1a}, at each occurrence, is independently hydrogen or C₁₋₈alkyl; and detecting a change in fluorescence intensity. In one aspect, the bridging ligands, or salts thereof, have a formula (Ia):

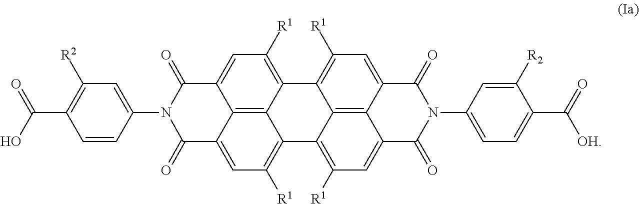

In another aspect, R¹, at each occurrence, is halo. In another aspect, R¹, at each occurrence, is chloro. In another aspect, R², at each occurrence, is C₁₋₈alkyl. In another aspect, R², at each occurrence, is C₁alkyl. In another aspect, R², at each occurrence, is hydrogen. In another aspect, the bridging ligands, or salts thereof, are selected from:

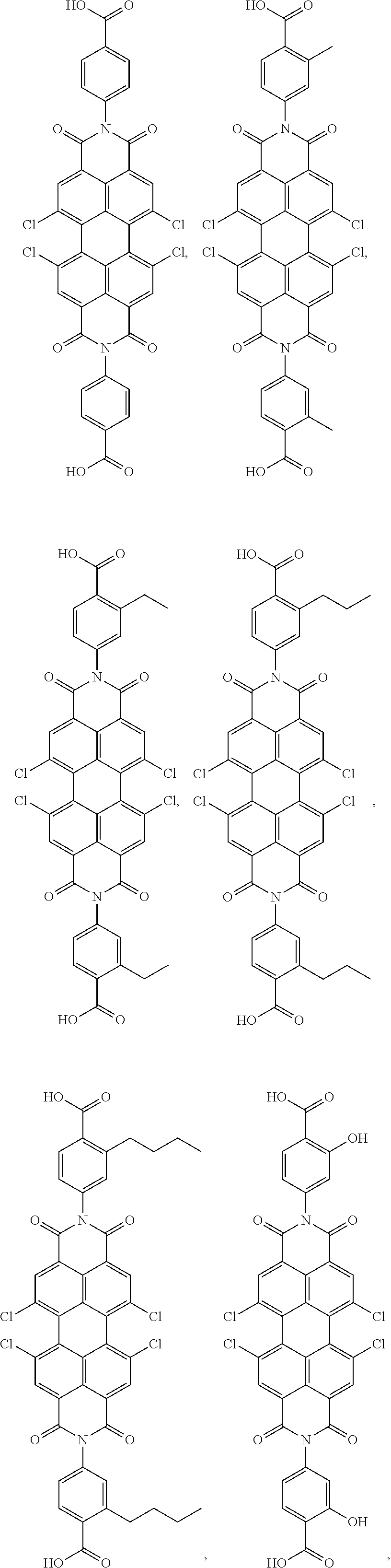

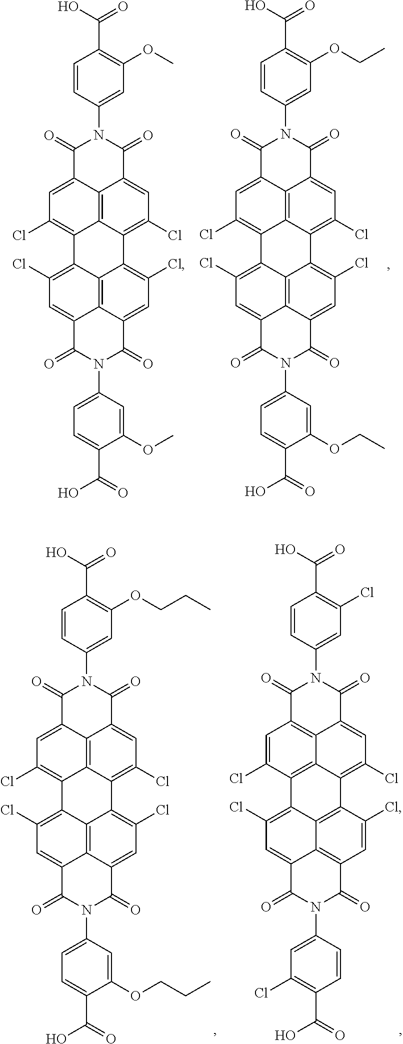

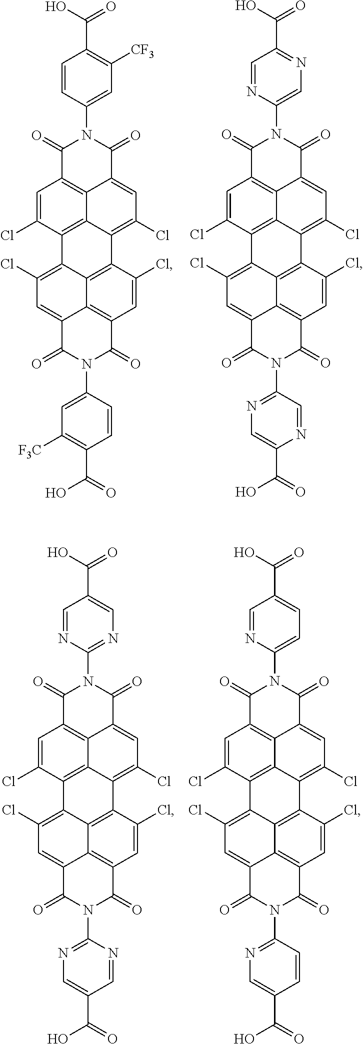

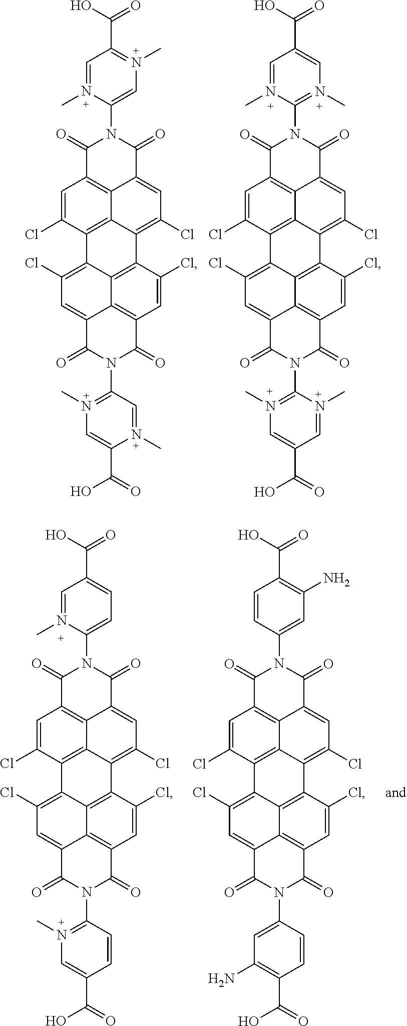

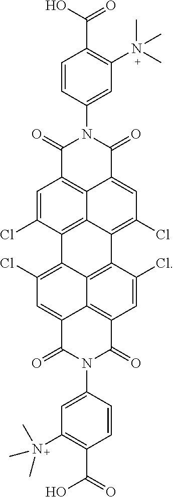

In another aspect, the secondary building units comprise one or more metal atoms or one or more carboxylates. In another aspect, the composition further comprises one or more modulators. In another aspect, the modulators are trifluoroacetic acid. In another aspect, the metal atoms comprise zirconium(IV) or hafnium(IV). In another aspect, the one or more perfluoroalkyl or polyfluoroalkyl substances is perfluooctanoic acid (PFOA). In another aspect, the composition is noncovalently adhered or covalently attached to a surface. In another aspect, the surface comprises cellulose, silica, a polymer, a copolymer, a resin, a filter, or a combination thereof. In another aspect, the composition is dissolved in a solvent. In another aspect, the change is an increase in fluorescence intensity. In another aspect, the change in fluorescence intensity indicate removal of the perfluoroalkyl or polyfluoroalkyl substances from the sample. In another aspect, the composition has a limit of detection of about 1-50 ppb.

Another embodiment described herein is a method for detecting or removing perfluoroalkyl or polyfluoroalkyl substances in a sample, the method comprising: contacting the sample with a composition comprising at least one metal-organic framework (MOF), or salt thereof, having a plurality of metal-containing secondary building units (SBU) linked together by one or more bridging ligands, wherein said bridging ligands, or salts thereof, have a formula (II):

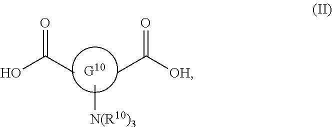

wherein
R¹⁰, at each occurrence, is independently hydrogen or C₁₋₈alkyl; and
G¹⁰ is independently a 6- to 12-membered aryl or a 5- to 12-membered heteroaryl. detecting a change in fluorescence intensity. In one aspect, R¹⁰, at each occurrence, is independently C₁alkyl. In another aspect, the bridging ligands have a formula (IIa):

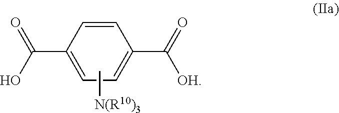

In another aspect, the bridging ligands, or salts thereof, are selected from:

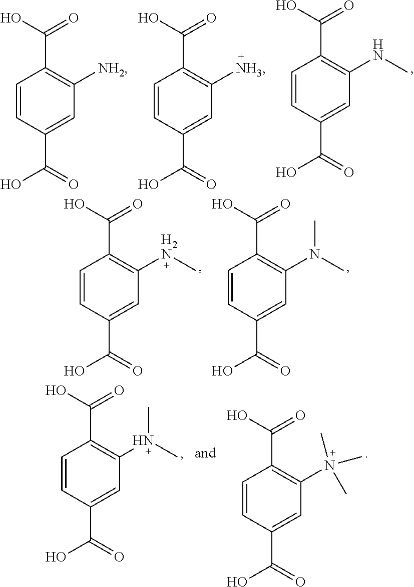

In another aspect, the secondary building units comprise one or more metal atoms and one or more carboxylates. In another aspect, the metal atoms comprise zirconium(IV) or hafnium(IV). In another aspect, the one or more perfluoroalkyl or polyfluoroalkyl substances is perfluooctanoic acid (PFOA). In another aspect, the composition is noncovalently adhered or covalently attached to a surface. In another aspect, the surface comprises cellulose, silica, a polymer, a copolymer, a resin, a filter, or a combination thereof. In another aspect, the composition is dissolved in a solvent. In another aspect, the change is an increase in fluorescence intensity. In another aspect, the change in fluorescence intensity indicates removal of perfluoralkyl or polyfluoroalkyl substances from the sample. In another aspect, the composition has a limit of detection of about 1-50 ppb.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1A shows the connection mode of Zr-based secondary binding unit clusters and molecular arrangement of PDI-based ligands (H₂L). FIG. 1B shows a-Axis crystal structure of U-1 MOF.

FIG. 2 shows ¹H NMR spectrum of H₂L ligand.

FIG. 3 shows ESI-MS spectrum of H₂L ligand.

FIG. 4 shows a plot for the Pawley refinement. U-1(exp) represents the measured data, U-1(sim) shows the simulated data, and difference indicates the difference between U-1(exp) and U-1(sim). The lines labeled as observed reflections display the allowed Bragg reflection positions.

FIG. 5 shows the asymmetric unit of U-1 MOF.

FIG. 6 shows the PDI core of H₂L ligand within crystals of U-1 MOF. The U-1 MOF in the absence of a PFAS is highly twisted. The ball-and-stick representation shows a dihedral angle of 36.9° associated with the bay carbon atoms C23-C24-C22-C32.

FIG. 7 shows XRPD patterns measured with the as-synthesized and activated U-1 MOF in comparison to the simulated patterns.

FIG. 8 shows the ATR-IR spectrum of U-1 MOF.

FIG. 9A shows an SEM image of a cluster of U-1 MOFs at lowest magnification. FIG. 9B shows an SEM imagine of a cluster of U-1 MOFs at higher magnification. FIG. 9C shows an SEM image of a single U-1 MOF at the highest magnification.

FIG. 10 shows nitrogen adsorption and desorption isotherms of U-1 MOF collected at −196° C.

FIG. 11 shows stability testing by XRPD measurement of the U-1 MOF sample treated under various conditions.

FIG. 12 shows the TGA curve of U-1 MOF measured in an air atmosphere in the temperature range of 20-700° C.

FIG. 13A shows a fluorescence microscope image of free bridging ligand. FIG. 13B shows a fluorescence microscope image of U-1 MOF.

FIG. 14 shows the solid-state emission behavior of free bridging ligand and U-1 MOF.

FIG. 15 shows the UV-Vis spectra of U-1 dispersed in DMF/water binary solution.

FIG. 16 shows the normalized fluorescence spectra recorded for free H₂L ligand and U-1 MOF dispersed in solution.

FIG. 17 shows the normalized fluorescence spectra recorded for U-1 MOF in solid form and dispersed in solution.

FIG. 18 shows the fluorescence emission spectra of U-1 MOF in solvents with different polarity.

FIG. 19 shows the effect of UV irradiation on the emission intensity of U-1 MOF in dispersed state.

FIG. 20 shows the fluorescence spectra of U-1 MOF suspension (0.13 mg/mL) measured in a binary solvent of water/DMF (60/40, v/v) in the presence of increasing concentration of PFOA (excitation wavelength lex: 490 nm). The inset shows the naked-eye visualization of the fluorescence emission change of U-1 MOF in a cuvette in the presence of PFOA (1 mM).

FIG. 21 shows the UV-Vis absorption spectra of U-1 MOF before and after sensing of PFOA.

FIG. 22A shows the fluorescence spectra of U-1 MOF before addition of PFOA. FIG. 22B shows the fluorescence spectra of U-1 MOF after addition of PFOA. FIG. 22C shows the UV-Vis spectra of U-1 MOF before addition of PFOA. FIG. 22D shows the UV-Vis spectra of U-1 MOF before addition of PFOA.

FIG. 23A shows the UV-Vis spectra of Rhodamine 6G. FIG. 23B shows the fluorescence spectra of Rhodamine 6G.

FIG. 24 shows time dependent change in emission intensity of U-1 MOF suspension with varying concentrations of PFOA.

FIG. 25 shows the relative fluorescence intensity change of U-1 MOF toward PFOA in comparison to other potential interferents (PFOS, octanoic acid, lauric acid, TFA, CTAB) as tested individually. The test was performed in binary solvent of water/DMF (60/40, v/v) and the concentrations of U-1 MOF, PFOA, and interferents are, 0.13 mg/mL, 0.3 mM, and 0.3 mM, respectively.

FIG. 26 shows the relative fluorescence intensity change of U-1 MOF toward PFOA in comparison to other potential interferents as tested with the interferents (PFOS, octanoic acid, lauric acid, TFA, CTAB). The test was performed in binary solvent of water/DMF (60/40, v/v), and the concentrations of U-1 MOF, PFOA and interferents are, 0.13 mg/mL, 0.3 mM, and 0.3 mM, respectively.

FIG. 27 shows the change in the emission spectra of U-1 suspensions in the presence of different analytes.

FIG. 28 shows the change in the fluorescence spectra of suspended U-1 upon addition of 0.3 mM PFOA in the presence of equal amounts of different competing analytes.

FIG. 29 shows the relative fluorescence intensity change of U-1 MOF toward PFOA in comparison to other potential PAHs interferents when tested individually.

FIG. 30 shows the change in the fluorescence spectra of the suspended U-1 MOF upon addition of 0.3 mM PFOA in the presence of equal amounts of naphthalene as interference. The inset shows a fluorescence photograph showing the physical change in emission observed under the UV lamp.

FIG. 31 shows the change in the fluorescence spectra of suspended U-1 upon addition of 0.3 mM PFOA in the presence of equal amounts of anthracene as interference.

FIG. 32 shows the change in the fluorescence spectra of suspended U-1 MOF upon addition of 0.3 mM PFOA in the presence of equal amounts of a 9, 10-phenanthrenequinone as interference.

FIG. 33 shows the change in the fluorescence spectra of suspended U-1 MOF upon addition of 0.3 mM PFOA in the presence of equal amounts of a Fluorene as interference.

FIG. 34 shows the change in the fluorescence spectra of suspended U-1 MOF upon addition of 0.3 mM PFOA in the presence of equal amounts of 2-Bromofluorene as interference.

FIG. 35A shows the change in the fluorescence spectra of suspended U-1 MOF in the presence of pyrene. FIG. 35B shows the change in the fluorescence spectra of suspended U-1 MO upon addition of 0.3 mM PFOA in the presence of equal amounts of a pyrene as interference.

FIG. 36 shows the relative fluorescence intensity change of U-1 MOF toward PFOA in presence of other PAHs as the interferents.

FIG. 37A show the change in the fluorescence spectra of suspended U-1 MOF upon addition of 0.3 mM PFOA in the presence of equal amounts of 3,5-dichlorobenzoic acid as an interferent. FIG. 37B show the change in the fluorescence spectra of suspended U-1 MOF upon addition of 0.3 mM PFOA in the presence of equal amounts of 2,4,6-Trichlorobenzoic acid as an interferent.

FIG. 38A shows the change in the fluorescence spectra of suspended U-1 MOF upon addition of 0.3 mM PFOA in the presence of equal amounts of 4-nitrophenol as an interferent.

FIG. 38B shows the change in the fluorescence spectra of suspended U-1 MOF upon addition of 0.3 mM PFOA in the presence of equal amounts of 4-nitrotoluene as an interferent.

FIG. 39 shows the fluorescence emission spectra of U-1 MOF in differing ratios of water/DMF.

FIG. 40 shows a bar plot showing the change in relative fluorescence intensity of U-1 after five cycles of PFOA sensing.

FIG. 41 shows the emission intensity measured for U-1 MOF dispersed in water/DMF (60/40 v/v) at concentration of 0.13 mg/mL as a function of the concentration of PFOA present.

FIG. 42 shows the titration performed for U-1 MOF deposited on a 1×1 cm filter paper (at a loading density of 5 mg/cm²) upon drop-casting of different concentrations of PFOA at a fixed volume of 75 μL.

FIG. 43 shows the fluorescence spectra of U-1 MOF suspension measured in a binary solvent of water/DMF in the presence of increasing concentrations of aqueous PFOA solutions made in drinking water.

FIG. 44 shows the fluorescence spectra of U-1 MOF suspension measured in a binary solvent of water/DMF in the presence of increasing concentrations of aqueous PFOA solutions made in tap water.

FIG. 45 shows the emission intensity measured for U-1 MOF dispersed in water/DMF (60/40 v/v) as a function of the concentration of PFOA present in drinking water.

FIG. 46 shows the emission intensity measured for U-1 MOF dispersed in water/DMF (60/40 v/v) as a function of the concentration of PFOA present in tap water.

FIG. 47 shows the XRPD patterns of U-1 MOF before and after the sensing experiment with PFOA.

FIG. 48A shows an SEM image of U-1 MOF before PFOA sensing. FIG. 48B shows an SEM image of U-1 MOF after PFOA sensing.

FIG. 49 shows the ¹⁹F NMR spectra of the digested MOF after experiment with PFOA.

FIG. 50 shows ¹⁹F NMR spectrum of PFOA.

FIG. 51A shows the XPS analysis for U-1 and PFOA@U-1 Zr 3d spectra before PFOA sensing. FIG. 51B shows the XPS analysis for U-1 and PFOA@U-1 Zr 3d spectra after PFOA sensing. FIG. 51C shows the F is spectra before PFOA sensing. FIG. 51D shows the F is spectra after PFOA sensing.

FIG. 52A shows the fluorescence spectra of suspended U-1 MOF upon addition of 0.3 mM of other acids. FIG. 52B shows the fluorescence spectra of suspended U-1 MOF upon addition of 0.3 mM of other acids as a bar plot.

FIG. 53 shows the PXRD pattern of U-1 MOF before and after PFOA interactions.

FIG. 54 shows the asymmetric unit of PFOA@U-1 MOF.

FIG. 55 shows the plot for the Pawley refinement of PFOA@U-1 MOF.

FIG. 56 shows the PDI core of H₂L ligand within crystals of PFOA@U-1 MOF represented by a ball-and-stick model. The model shows a dihedral angle of 26.4° associated with the bay carbon atoms C23-C24-C22-C32.

FIG. 57 shows the optimized structure PFOA@U-1 after DFT calculation.

FIG. 58A shows the DFT-optimized geometries of the PFOA-PDI ligand-Zr₆ node domains within PFOA@U-1 are depicted at the adsorption site. FIG. 58B shows a magnified view of the respective adsorption site shows dashed lines representing hydrogen bonding and covalent interactions of RCOOH . . . F—C and RCOOH—Zr₆ motifs, respectively.

FIG. 59 shows an ¹⁹F NMR spectrum of TFA and PFOA before adsorption.

FIG. 60A shows the structure of coordinated zinc. FIG. 60B shows the crystal structure of UiO-66 type MOF.

FIG. 61 shows powder XRD at different stages of modifications of UiO-66-based MOFs.

FIG. 62 shows BET surface area of UiO-66-based MOFs.

FIG. 63 shows the ¹H NMR patterns of linkers present in UiO-66-based MOF.

FIG. 64 shows the PFOA uptake capacity of different absorbent materials at room temperature.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various way.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

As used herein “or” can mean the conjunctive or disjunctive. For example, two actions separated by an “or” can be performed either simultaneously, sequentially, or separately.

It will be understood that when a feature, such as a layer, region or substrate, is referred to as being “on” another feature or element, it can be directly on the other feature or element or intervening features and/or elements may also be present. In contrast, when an element is referred to as being “directly on” another feature or element, there are no intervening elements present.

As used herein, the term “complex” and “composition” may be used interchangeably.

As used herein, the term “coordinated” or “coordination” describes the interaction between a metal atom and a ligand, including but not limited to atoms, ions, or molecules. As used herein, coordination and “link” or “linking” may be used interchangeably.

As used herein, the term “non-covalently adhered” describes interactions between atom, ions, and molecules that do not involve the sharing of electrons.

As used herein, the term “covalently attached” describes interactions between atoms, ions, and molecule that do involve the sharing of electrons.

As used herein, the term “bind” or “bound” may refer to either or both covalent and non-covalent interactions.

### A. Metal-Organic Frameworks

As used herein, the term “metal organic framework” or “MOF”, refers to a complex comprising one or more secondary building units and one or more bridging ligands. In some embodiments, the complex further comprises one or more modulators. In one aspect, the modulators are trifluoroacetic acid. In another aspect, the modulators are weakly bound to the secondary building units, the bridging ligands, or a combination of the bridging ligand and the secondary building units. In another aspect, the modulators maintain the coordination between the secondary building units and bridging ligands together in the absence of an analyte. In another aspect, the presence of an analyte displaces trifluoroacetic acid and the analyte binds to the location where trifluoroacetic acid previously existed.

As used herein, “secondary building units” also referred to as SBU, refers to the component of the MOF comprising one or more metal atoms and one or more complexing agent. The “base” of the secondary building units, as described herein, is the one or more metal atoms. In some embodiments, the metal atoms are linked by bridging oxygen or hydroxyl groups. In some embodiments, the metal atoms are further coordinated to at least one bridging ligand. In some embodiments, the one or more complexing agent is a sulfate or carboxylate salt. In some embodiments, the carboxylate salt is formate, benzoate, or acetate.

MOFs generally are known in the art and encompass structures in which a metal base is coordinated to an at least bidentate bridging ligands to form a coordination network. Such structures may be one-dimensional, two-dimensional, or three-dimensional. The MOFs of the present disclosure are porous, and may include pores that occur in the pores between the metal ions and the coordination network of the one or more bridging ligands. The pores may be micropores, 2 nm or less in diameter, or mesopores, 2-50 nm in diameter.

A number of methods for producing MOFs are known in the art. The most common technique involves the reaction of a metal salt with the desired bridging ligand in a suitable solvent, typically an organic solvent such as Dimethylformamide (DMF). High pressures and temperatures are generally required to facilitate the reaction. Typical processes are disclosed in, for example, WO 2009/133366, WO 2007/023134, WO2007/090809 and WO 2007/118841.

In some embodiments, the one or more metal atoms may be zirconium, hafnium, titanium, or cerium. In some embodiments, the one or more atom metals ions may be present in an amount of at most 50 wt. %, preferably at most 25 wt. %, more preferably at most 10 wt. %, for example at most 5 wt. %, relative to the total amount of metal ions.

The bridging ligands described herein are capable of being coordinated to at least two metal atoms through at least one organic moiety. As described herein an “organic moiety” means a carbon-based group that includes at least one C—H bond and which may optionally include one or more heteroatoms such as N, O, S, B, P, Si. Typically, the organic moiety comprises from 1 to 75 carbon atoms. In some embodiments, the bridging ligand is the ligand itself or a salt thereof.

In some embodiments, the bridging ligand is at least bidentate. As described herein “bidentate” refers to two functional groups capable of basic coordination with a metal atom. The bridging ligand compound may also be tridentate (i.e., containing three functional groups) or tetradentate (i.e., containing four functional groups). In some embodiments, the bridging ligand coordination involves the supplying of electron from the bridging ligand to the base.

In some embodiments, the bridging ligand may be water soluble. Water solubility, as described herein, refers to solubility in water which is high enough to ensure that a homogeneous solution is formed in water. The solubility of the organic linker compound in water may be at least 1 g/L, such as at least 2 g/L, or at least 5 g/L at Room Temperature and Pressure (RTP).

In some embodiments, the bridging ligand comprises an aromatic moiety. The aromatic moiety may have one or more aromatic rings, for example two-, three-, four-, five-, six-, seven-, eight-, nine-, or ten rings, which can be present separately from each other and/or at least two rings may be present in a fused form. In some embodiment, each ring of the moiety may independently include at least one heteroatom, such as N, O, S, B, P, Si, preferably N, O and/or S. In some embodiments, the bridging ligand comprises a perylene diimide. In some embodiments, the perylene diimide is substituted with other functional groups.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C1-6alkyl” means a straight or branched chain hydrocarbon containing from 1 to 6 carbon atoms. The term “C1-4alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl” as used herein, means at least one amino group, as defined herein, is appended to the parent molecular moiety through an alkylene group, as defined herein.

The term “amino,” as used herein, means —NRxRy, wherein Rx and Ry may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRx-, wherein Rx may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For illustration, an example cycloalkylene may be cyclohexene or

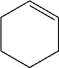

and a heterocyclylene may be

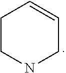

Cycloalkylene and heterocyclylene include a geminal divalent groups such as 1,1-C₃₋₆cycloalkylene. A further example is 1,1-cyclopropylene.

The term “halogen” or “halo,” as used herein, means C1, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g., 1, 2, 3, or 4 heteroatoms independently selected from O, S, and N). The five membered aromatic monocyclic rings have two double bonds, and the six membered aromatic monocyclic rings have three double bonds. The bicyclic heteroaryl is an 8- to 12-membered ring system and includes a fused bicyclic heteroaromatic ring system (i.e., 1071 electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 107L electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

Terms such as “alkyl,” “cycloalkyl,” “alkylene,” etc. may be preceded by a designation indicating the number of atoms present in the group in a particular instance (e.g., “C1-4alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

The bridging ligands described herein are set forth in the following numbered embodiments. The first embodiment is denoted E1, another embodiment is denoted E2 and so forth.

E1. A bridging ligand of formula (I), or salt thereof:

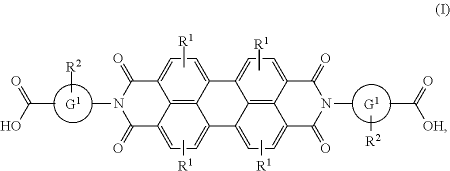

wherein
R¹, at each occurrence, is independently hydrogen, C₁₋₈alkyl, halo, —O(R^{1a}), —N(R^{x})ₘ, or —S(O)₂OH;
R², at each occurrence, is independently hydrogen, C₁₋₈alkyl, —O(R^{1a}), or C₁₋₈alkyl-OH;
G¹, at each occurrence, is independently phenyl, a 6- to 12-membered aryl, or a 5- to 12-membered heteroaryl,
R^{1a}, at each occurrence, is independently hydrogen or C₁₋₈alkyl, or phenyl;
R^{x}, at each occurrence, is independently hydrogen or C₁₋₃alkyl; and
m=2-3.

E2. The bridging ligand of E1, or salt thereof, wherein G¹, at each occurrence, is phenyl.

E3. The bridging ligand of E1 or E2, or salt thereof, have a formula (Ia):

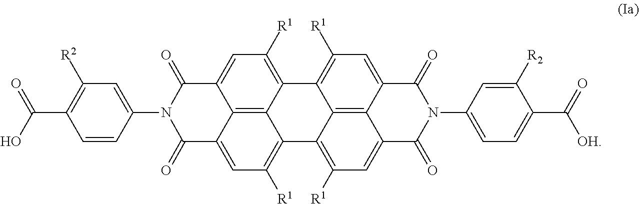

E4. The bridging ligand of any one of E1-3, or salt thereof, wherein R¹, at each occurrence, is halo.

E5. The bridging ligand of any one of E1-4, or salt thereof, wherein R¹, at each occurrence, is chloro.

E6. The bridging ligand of any one of E1-5, or salt thereof, wherein R², at each occurrence, is C₁₋₈alkyl.

E7. The bridging ligand of any one of E1-6, or salt thereof, wherein R², at each occurrence, is C₁alkyl.

E8. The bridging ligand of any one of E1-7, or salt thereof, wherein every R² is not hydrogen.

E9. The bridging ligand of any one of E1-8, or salt thereof, wherein the bridging ligand, or salts thereof, is selected from:

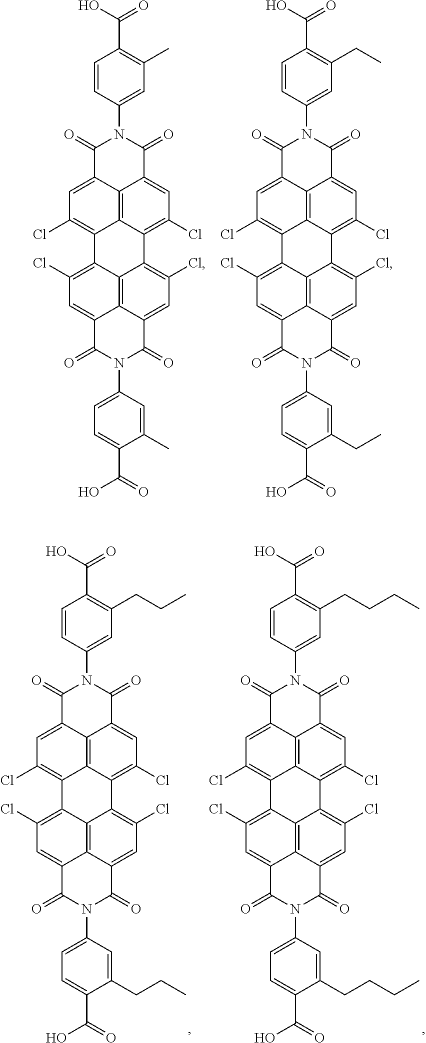

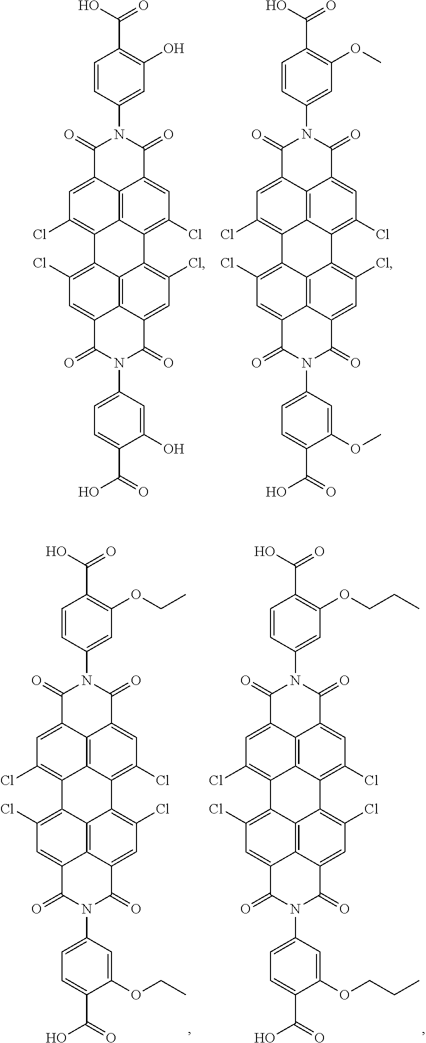

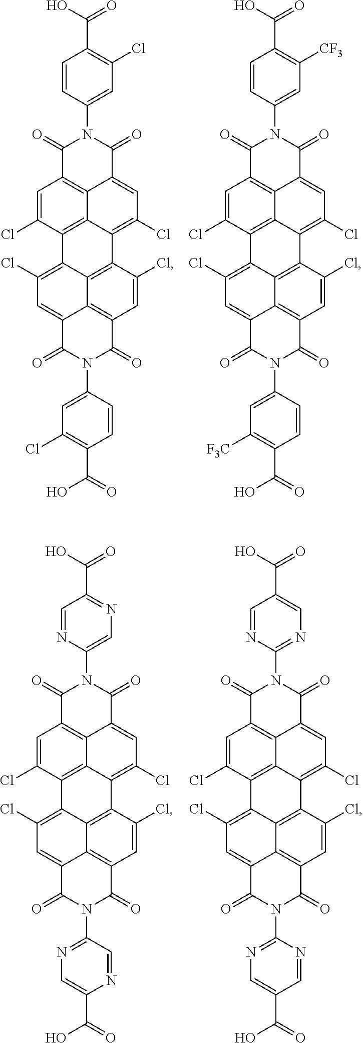

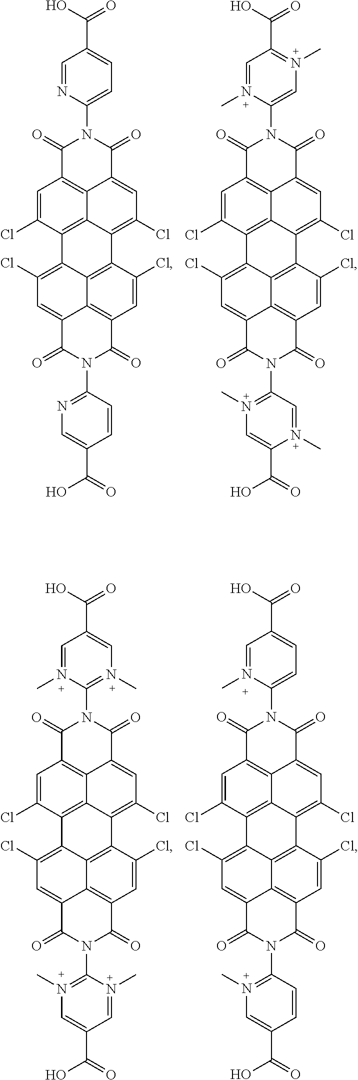

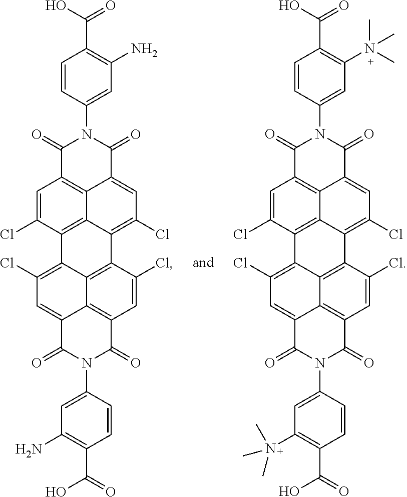

E10. A bridging ligand of formula (II), or salt thereof:

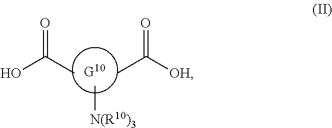

wherein
R¹⁰, at each occurrence, is independently hydrogen or C₁₋₈alkyl; and
G¹⁰ is independently a 6- to 12-membered aryl or a 5- to 12-membered heteroaryl.

E11. The bridging ligand of E10, wherein the bridging ligands have a formula (IIa):

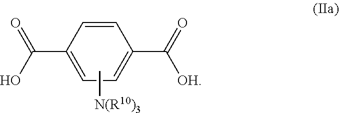

E12. The bridging ligand of E10 or E11, wherein the bridging ligand, or salt thereof, is selected from:

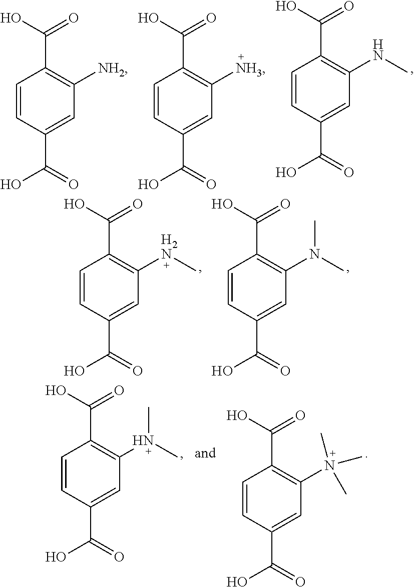

In some embodiments, the MOF containing the bridging ligand described herein is selective or non-selective for one or more perfluoroalkyl or polyfluoroalkyl substances. In some embodiments, the limit of detection is about 1 to about 50 parts per billion (ppb). In one aspect, the limit of detection is about 1 ppb, 2 ppb, 3 ppb, 4 ppb, 5 ppb, 6 ppb, 7 ppb, 8 ppb, 9 ppb, 10 ppb, 15 ppb, 20 ppb, 25 ppb, 30 ppb, 35 ppb, 40 ppb, 45 ppb, 50 ppb, or greater. In another aspect, the limit of detection is about 1-10 ppb, 1-15 ppb, 1-20 ppb, 1-25 ppb, 1-30 ppb, 1-35 ppb, 1-40 ppb, 1-45 ppb, 1-50 ppb, 2-10 ppb, 2-15 ppb, 2-20 ppb, 2-25 ppb, 2-30 ppb, 2-35 ppb, 2-40 ppb, 2-45 ppb, 2-50 ppb, 5-10 ppb, 5-15 ppb, 5-20 ppb, 5-25 ppb, 5-30 ppb, 5-35 ppb, 5-40 ppb, 5-45 ppb, 5-50 ppb, 10-20 ppb, 10-25 ppb, 10-30 ppb, 10-35 ppb, 10-40 ppb, 10-45 ppb, 10-50 ppb, including all endpoints, integers and subranges within the disclosed ranges.

### B. Salts

The MOFs and bridging ligands described herein may exist as salts. In some embodiments the compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### C. Fluorescence and Fluorescence Quenching

In some embodiments, the organic ligand may possess fluorescence. In separate embodiments, the organic ligand may exhibit an increase in fluorescence in the presence of an analyte. As used herein, “fluorescence” is a cyclical process where a luminescence is generated by certain molecules in which the molecular absorption of a photon triggers the emission of another photon with a longer wavelength. Certain molecules are capable of being excited, via absorption of light energy, to a higher energy state, also called an excited state. The energy of this short-lived excited state decays (or decreases) resulting in the emission of light energy. The emission of light via this process is “fluorescence.” Molecules that emit light in this manner are said to “fluoresce” and are generally referred to as “fluorophores” or “fluorescent dyes.”.

A “fluorophore” or “fluorescent dye,” as used herein, is a molecule that is capable of fluorescing. In its ground state, the fluorophore molecule is in a relatively low-energy, stable configuration, and it does not fluoresce. When light from an external source of one or more particular wavelengths contacts a fluorophore, the fluorophore can absorb the light energy. If the fluorophore absorbs sufficient energy, the fluorophore is excited to an excited state (high energy); this process is known as excitation. There may be multiple excited states or high energy levels that a fluorophore can attain, depending on the wavelength and energy of the external light source. Since a fluorophore is unstable at high-energy configurations, it eventually decays to the lowest-energy excited state, which is semi-stable. The excited lifetime (the length of time that a fluorophore is an excited state) is very short; the fluorophore the decays from the semi-stable excited state back to the ground state, and at least a portion of the excess energy released by this decay may be emitted as light. The emitted light is of a lower energy, and a longer wavelength, than the absorbed light, and thus the color of the light that is emitted is different from the color of the light that has been absorbed. upon reaching the ground state, a fluorophore can again absorb light energy to enter an excited state.

A fluorophore or fluorescent dye absorbs light over a range of wavelengths and every dye has a characteristic range of excitation wavelengths. This range of excitation wavelengths is referred to as the fluorescence “excitation spectrum” and reflects the range of possible excited states that the dye can achieve. Certain wavelengths within this range are more effective for excitation than other wavelengths. A fluorophore is excited most efficiently by light of a particular wavelength. This wavelength is the excitation maximum for the fluorophore. As used herein “excitation maximum” refers to the specific wavelength for each fluorescent d_{y}e that most effectively induces fluorescence. Less efficient excitation can occur at wavelengths near the excitation maximum; however, the intensity of the emitted fluorescence is reduced. Although illumination at the excitation maximum of the fluorophore produces the greatest fluorescence output, illumination at lower or higher wavelengths affects only the intensity of the emitted light; the range and overall shape of the emission profile are unchanged.

As used herein, “excitation” refers to the process where a photon of energy supplied by an external source, such as a laser or a lamp, is absorbed by the fluorophore creating an excited electronic singlet state (S₁′) from the S₀ ground state. The excited state exists for a finite time during which the fluorophore undergoes conformational changes and is also subject to a multitude of possible interactions with its molecular environment. These processes have two important consequences. One of these consequences is that the energy of S₁′ is partially dissipated, yielding a relaxed singlet excited state (S₁) from which fluorescence emission originates. Molecules in an excited state (S₁′) can relax by various competing pathways, They can undergo ‘non-radiative relaxation’ in which the excitation energy is dissipated as heat (vibrations) to the solvent. Excited organic molecules can also relax via conversion to a triplet state which may subsequently relax via phosphorescence or by a secondary non-radiative relaxation step. The term “relax” as used herein refers to the energy loss of an excited molecule, Not all the molecules initially excited by absorption return to the ground state (S₀) by fluorescence emission. Relaxation of an S₁′ state can also occur through interaction with a second molecule through fluorescence quenching. Other processes, such as, but not limited to, collisional quenching or fluorescent resonance energy transfer (FRET), may also depopulate S₁.

FRET is now widely known in the art. FRET is a radiationless process in which energy is transferred from an excited donor molecule to an acceptor molecule. Radiationless energy transfer is the quantum-mechanical process by which the energy of the excited state of one fluorophore is transferred without actual photon emission to a second fluorophore or molecule. The quantum physical principles are reviewed in Jovin and Jovin, 1989, Cell Structure and Function by Microspectrofluorometry. eds. E, Kohen and J. C. G Hirschberg, Academic Press incorporated herein by reference for the teachings. Briefly, a fluorophore absorbs light energy at a characteristic wavelength. The first fluorophore is generally termed the donor (“D”) and my have an excited state of higher energy than that of the second fluorophore, termed the acceptor (“A”).

An essential feature of FRET is that the emission spectrum of the donor overlap with the excitation spectrum of the acceptor, and that the donor and acceptor be sufficiently close. In addition, the distance between “D” and “A” must be sufficiently small to allow the radiationless transfer of energy between the fluorophores. Because the rate of energy transfer is inversely proportional to the sixth power of the distance between the donor and acceptor, the energy transfer efficiency is extremely sensitive to distance changes. Energy transfer is said to occur with detectable efficiency in the 1-10 nm distance range but is typically 4-6 nm for optimal results. The distance range over which radiationless energy transfer is effective depends on many other factors as well, including the fluorescence quantum efficiency of the donor, the extinction coefficient of the acceptor, the degree of overlap of their respective spectra, the refractive index of the medium, and the relative orientation of the transition moments of the two fluorophores.

As used herein, the term “quencher” means a substance, which reduces or quenches the emission of fluorescence from a fluorophore. As used herein, “fluorescence quenching” may be achieved by any mechanism, typically by FRET between a fluorophore and a non-fluorescent quenching moiety or by collisional (i.e., contact) quenching.

Fluorophore molecules, when excited, emit over a range of wavelengths. This range of wavelengths is referred to as the fluorescence “emission spectrum.” There is a spectrum of energy changes associated with these emission events. The emission maximum is the wavelength where the population of molecules fluoresces most intensely. The emission maximum for a given fluorophore is always at a longer wavelength (lower energy) than the excitation maximum. This difference between the excitation and emission maxima is called the Stokes shift. The magnitude of the Stokes shift is determined by the electronic structure of the fluorophore and is characteristic of the fluorophore molecule. The Stokes shift occurs because some of the energy of the excited fluorophore is lost through molecular vibrations that occur during the brief lifetime of the molecule's excited state, which is dissipated as heat to surrounding solvent molecules as they collide with the excited fluorophore, Remaining energy that is emitted as light fluorescence is thus less than the amount of energy required for excitation.

Fluorescence requires a source of excitation energy, There are many light source options for fluorescence, Selecting the appropriate light source, and filters for both excitation and emission, can increase the sensitivity of signal detection.

Several types of light sources are used to excite fluorescent dyes. The most common sources used are broadband sources, such as, for example, mercury-arc and tungsten-halogen lamps. These lamps produce white light that has peaks of varying intensity across the spectrum. When using broadband white light sources, it is necessary to filter the desired wavelengths needed for excitation; this is most often done using optical filters. Optical filters selectively allow light of certain wavelengths to pass while blocking out undesirable wavelengths. A bandpass excitation filter transmits a narrow range of wavelengths and may be used for selective excitation.

Laser excitation sources provide wavelength peaks that are well-defined, selective, and of high intensity allowing more selective illumination of the sample. The best performance is achieved when the dye's peak excitation wavelength is close to the wavelength of the laser. Several lasers commonly used include, for example, the compact violet 405 nm laser, 488 nm blue-green argon-ion laser, 543 nm helium-neon green laser, and 633 nm helium-neon red laser, Mixed-gas lasers such as, for example, the krypton-argon laser, can output multiple laser lines which may require optical filters to achieve selective excitation. High-output light-emitting diodes (LEDs) provide selective wavelengths, low cost and energy consumption, and long lifetime. Single-color LEDs are ideal for low-cost instrumentation where they can be combined with simple long-pass filters that block the LED excitation and allows the transmission of the dye signal. However, the range of wavelengths emitted from each LED is still relatively broad and may also require the use of a filter to narrow the bandwidth.

Filters are important for selecting excitation wavelengths and for isolating the fluorescence emission emanating from the dye of interest, Stray light arising from sources other than the emitting fluorophores (for example, from the excitation source) interferes with the detection of the fluorescence emission. Stray light therefore must be contained to ensure only the fluorescence of the sample registers with the instrument's light-sensitive detectors. When a single fluorophore is used, a long pass emission filter which selectively blocks out the excitation light to reduce background noise nay be used to maximize the signal collected. If multiple fluorophores are used in the sample, a band pass emission filter can be used to isolate the emission from each dye.

### D. Solid Phase Extraction

In some embodiments, the MOF may be covalently attached or non-covalently adhered to a surface. In some embodiments, the surface is a “solid phase extraction membranes” or “SPE membranes.” As used herein, “surface” and “solid-phase extraction membrane” may be used interchangeably. As used herein a surface may comprise a polymer, a copolymer, a resin, a filter, silica, or a combination thereof. In some embodiments, the polymer may be selected from a polytetrafluoroethylene, a polyvinylidene difluoride, a nitrocellulose, a cellulose acetate, a polyacrylonitrile, a polyimide, a polycarbonate, and a polypropylene, among other suitable polymers. In some embodiments, the surface may comprises a polymer forming a porous polymeric aggregate or polymeric fibers with microparticles and/or nanoparticles embedded in the polymer. A “microparticle” or “nanoparticle” used herein refers to a plurality of particles that either adsorb or absorb liquids. In some embodiments the microparticle or nanoparticle may be formed of a silica, an activated carbon, a cation exchange resin, an anion exchange resin, a chelating agent, a polystyrenedivinylbenzene, or a reverse phase sulfonated polystyrenedivinylbenzene, among other suitable materials. In some embodiments, the microparticles or nanoparticles are C8 or C18 bonded silica. In some embodiments, the average particle size of the solid phase extraction membrane may be from about 0.5 μm to about 20 μm. In some embodiments, the average particle size may be from about 5 μm to about 15 μm. In some embodiments, the average particle size may be from 10 μm to about 15 μm. In some embodiments, the pore size of the SPE membrane is about 0.1 μm to about 10 μm. In some embodiments, the SPE membrane may be housed in a disk or other compatible apparatus. When the SPE membrane is housed in a disk, the diameter of the disk may range from about 5 mm to about 100 mm. In some embodiments, the SPE membrane may exist separate from a disk to allow for fluorescence imaging.

In some embodiments, an organic linker or organic ligand, as described herein, may be covalently or non-covalently adhered on a surface or a solid-phase extraction (“SPE”) membrane. In some embodiments, the organic ligand may be covalently coupled to a surface and resulting fluorescence is detected directly on the surface. For example, the organic ligand may be covalently attached to the surface by a linker, including any linker to those skilled in the art or hereinafter developed. The linker may be formed by reaction of one or more functional groups on the surface and one or more functional groups on the organic linker or ligand. Exemplary linking chemistry may include but is not limited to modifying the silica gel surface with amino-alkyl-trimethoxysilane, or amino-alkyl-triethoxysilane, to form an activated silica comprising an amine. Subsequently tethering the organic ligand via reaction with the amine moiety of the activated silica. The —OH, —COOH, or —NH2 groups on the surface of polymer or silica can be strongly bound to with the metal salt used for MOF synthesis (Zr4+) through coordination. For example, the polymer or silica may be treated with the metal salt alone to form a metal ion coating on the surface as a nucleation site for MOF. In the next step, both the metal salt and ligand are added and grow MOFs on the surface of the polymer or silica support by following standard solvothermal reaction conditions used for MOF synthesis. Since the metal ion is already attached to the surface of the support, the MOF starts growing on the surface. In some embodiments, the organic linker or ligand may be non-covalently adhered to the surface, such as via intramolecular interactions including, but not limited to, electrostatic interactions, 7-effects interactions, hydrogen bonding, van der Waals forces, and/or hydrophobic interactions. In some embodiments, either together or individually the organic linkers aid the secondary building unit (SBU) form non-covalent interactions with the surface during the formation of the MOF and facilitate the formation of the MOF on the surface.

### E. Analyte Detection

The sample comprising the analyte may be prepared experimentally or obtained from the natural environment. In some embodiments, the sample may be obtained from a river, lake, well, pond, stream, ocean, inlet, canal, loch, bay, fountain, sea, or a combination thereof. In other embodiments, the sample may be obtained from tap water, municipal water, municipal wastewater, industrial wastewater, run-off water, ballast water, water treatment water, agricultural water, or a combination of. A liquid sample comprising the analyte can be prepared by treating a solid sample with a liquid to dissolve an analyte. In some embodiments, the solid may be soil, sand, rock, or a combination thereof.

It will be apparent to those of ordinary skill in the relevant art that suitable modifications and adaptations to the methods, processes, and applications described herein can be made without departing from the scope of any embodiments or aspects thereof. The methods provided are exemplary and are not intended to limit the scope of any of the specified embodiments. All of the various embodiments, aspects, and options disclosed herein can be combined in any variations or iterations. The scope of the methods and processes described herein include all actual or potential combinations of embodiments, aspects, options, examples, and preferences herein described. The exemplary methods described herein may substitute any component disclosed herein, or include any component disclosed elsewhere herein. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A composition comprising at least one metal-organic framework (MOF), or salt thereof, the metal-organic framework comprising:
   a plurality of metal-containing secondary building units (SBU) linked together by one
   or more bridging ligands,
      wherein said bridging ligands, or salts thereof, have a formula (I):
         a.

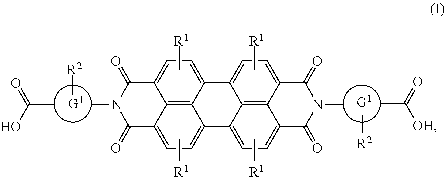

wherein
R¹, at each occurrence, is independently hydrogen, C₁₋₈alkyl, halo, —O(R^{1a}), —N(R^{x})ₘ, or —S(O)₂OH;
R², at each occurrence, is independently hydrogen, C₁₋₈alkyl, —O(R^{1a}), or
C₁₋₈alkyl-OH and wherein every R² is not hydrogen;
G¹, at each occurrence, is independently phenyl, a 6- to 12-membered aryl, or a 5- to 12-membered heteroaryl, wherein the 5- to 12-membered heteroaryl is optionally substituted at a heteroatom with hydrogen or C₁₋₈alkyl;
R^{1a}, at each occurrence, is independently hydrogen or C₁₋₈alkyl, or phenyl;
R^{x}, at each occurrence, is independently hydrogen or C₁₋₃alkyl; and
m=2-3.
Clause 2. The compositions of clause 1, wherein G¹, at each occurrence, is phenyl.
Clause 3. The composition of clauses 1 or 2, wherein the bridging ligands, or salts thereof, have a formula (Ia):

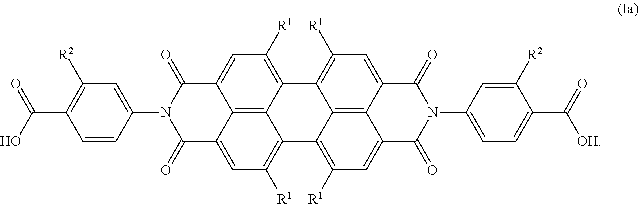

Clause 4. The composition of any one of clauses 1-3, wherein R¹, at each occurrence, is halo.
Clause 5. The composition of any one of clauses 1-4, wherein R¹, at each occurrence, is chloro.
Clause 6. The composition of any one of clauses 1-5, wherein R², at each occurrence, is C₁₋₈alkyl.
Clause 7. The composition of any one of clauses 1-6, wherein R², at each occurrence, is C₁alkyl.
Clause 8. The composition of any one of clauses 1-7, wherein the bridging ligands, or salts thereof, are selected from:

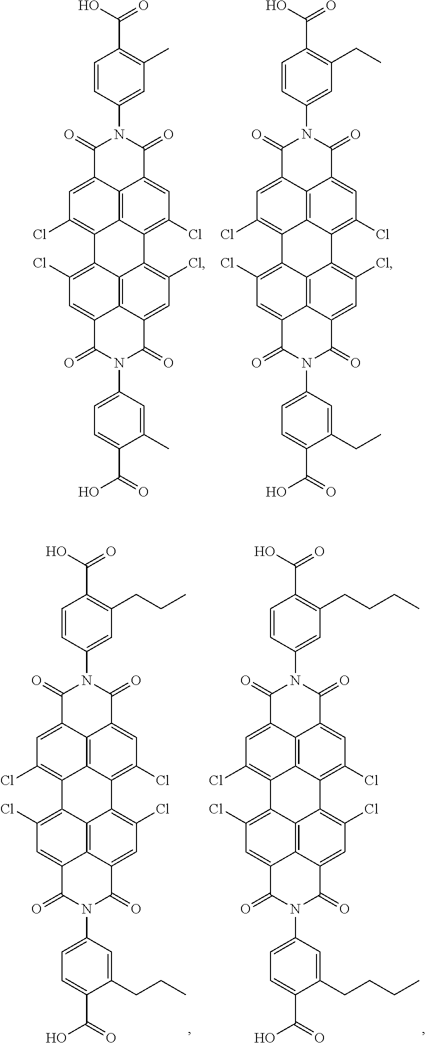

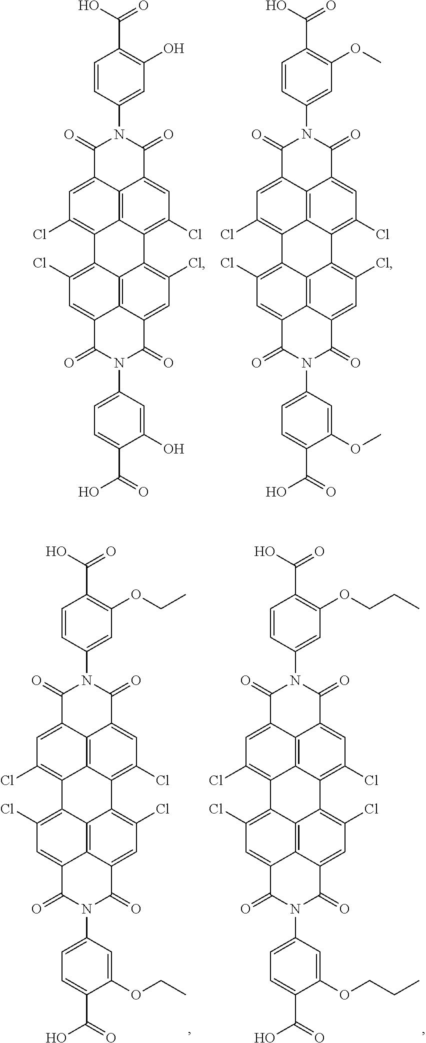

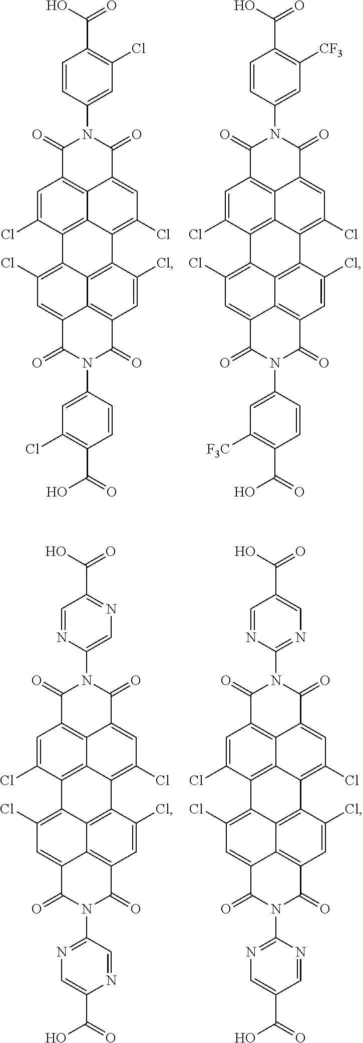

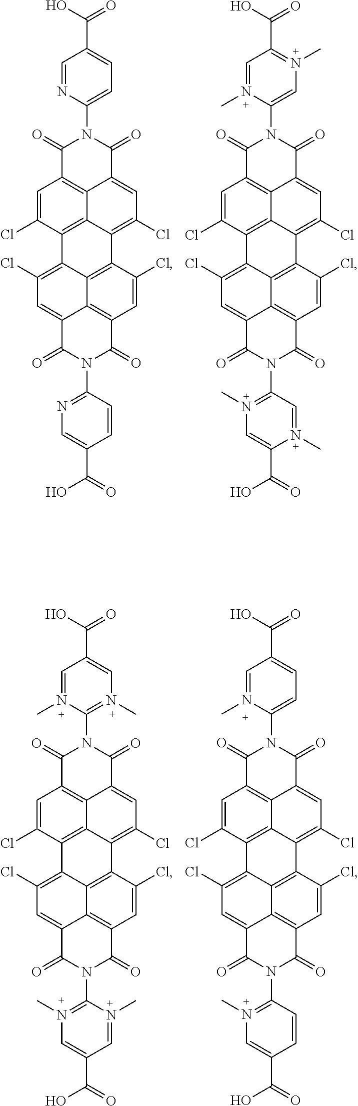

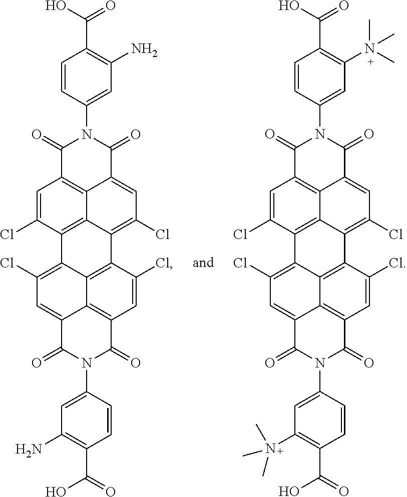

Clause 9. The composition of any one of clauses 1-8, wherein the secondary building units comprise one or more metal atoms and one or more carboxylates.
Clause 10. The compositions of any one of clauses 1-9, wherein the metal atoms comprise zirconium(IV) or hafnium(IV).
Clause 11. The composition of any one of clauses 1-10, wherein the composition further comprises one or more modulators.
Clause 12. The composition of any one of clauses 1-11, wherein the modulators are trifluoroacetic acid.
Clause 13. A method for detecting or removing perfluoroalkyl or polyfluoroalkyl substances in a sample, the method comprising:
   contacting the sample with a composition comprising at least one metal-organic framework (MOF), or salt thereof, having a plurality of metal-containing secondary building units (SBU) linked together by one or more bridging ligands,
      wherein said bridging ligands, or salts thereof, have a formula (I):

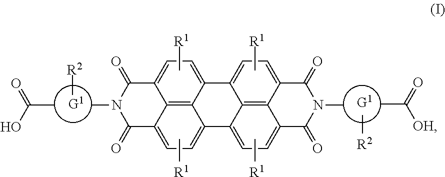

wherein
R¹, at each occurrence, is independently hydrogen, C₁₋₈alkyl, or halo;
R², at each occurrence, is independently hydrogen, C₁₋₈alkyl, —O(R^{1a}), or C₁₋₈alkyl-OH; and
R^{1a}, at each occurrence, is independently hydrogen or C₁₋₈alkyl; and detecting a change in fluorescence intensity.
Clause 14. The method of clause 13, wherein the bridging ligands, or salts thereof, have a formula (Ia):

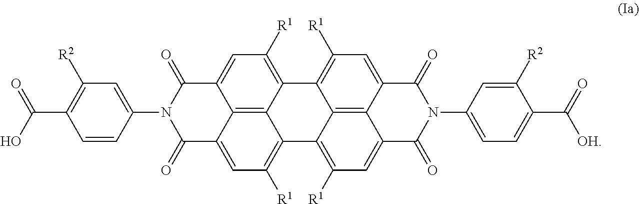

Clause 15. The method of clauses 13 or 14, wherein R¹, at each occurrence, is halo.
Clause 16. The method of any one of clauses 13-15, wherein R¹, at each occurrence, is chloro.
Clause 17. The method any one of clauses 13-16, wherein R², at each occurrence, is C₁₋₈alkyl.
Clause 18. The method of any one of clauses 13-17, wherein R², at each occurrence, is C₁alkyl.
Clause 19. The method of any one of clauses 13-18, wherein R², at each occurrence, is hydrogen.
Clause 20. The method of any one of clauses 13-19, wherein the bridging ligands, or salts thereof, are selected from:

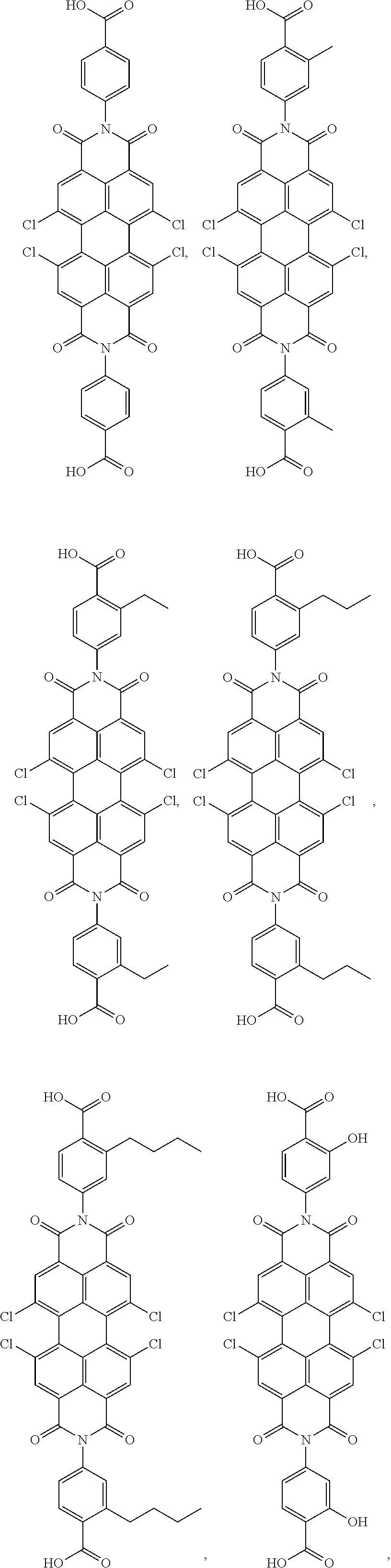

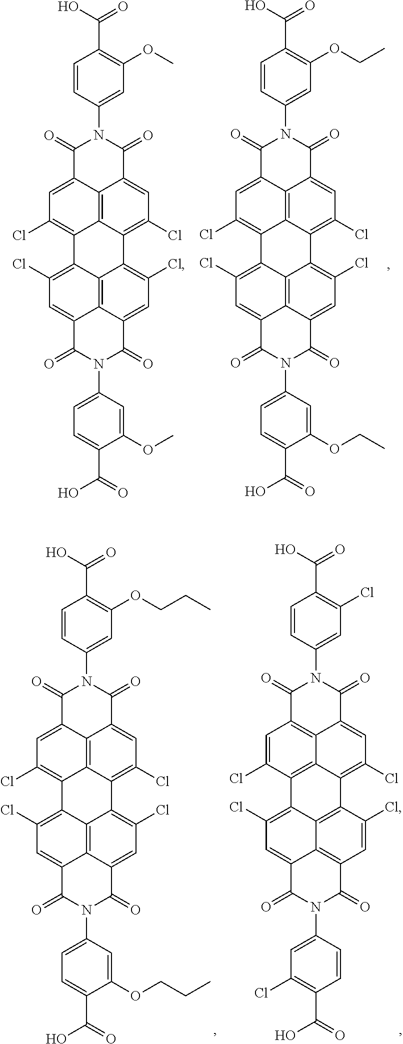

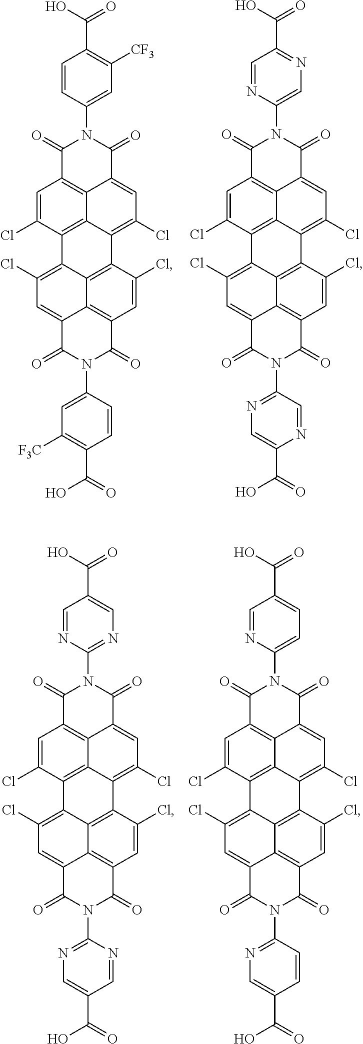

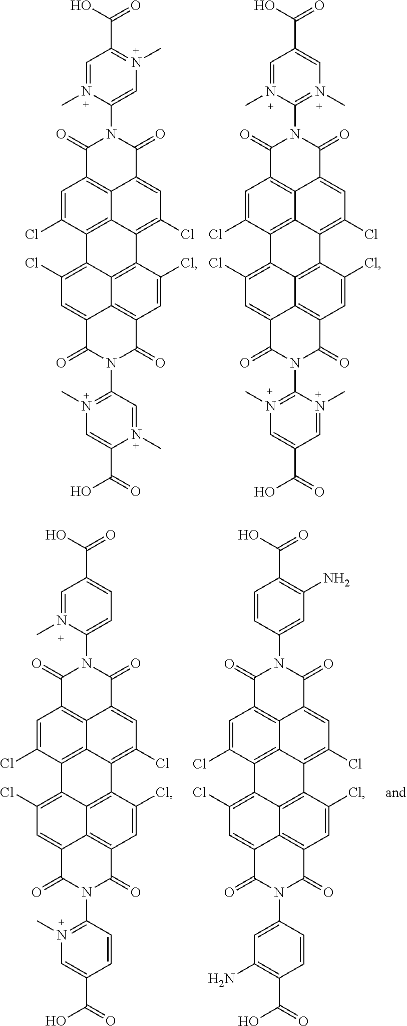

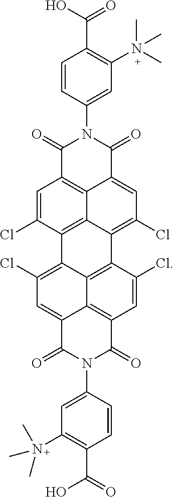

Clause 21. The method of any one of clauses 13-20, wherein the secondary building units comprise one or more metal atoms or one or more carboxylates.
Clause 22. The method of any one of clauses 13-21, wherein the metal atoms comprise zirconium(IV) or hafnium(IV).
Clause 23. The method of any one of clauses 13-22, wherein the composition further comprises one or more modulators.
Clause 24. The method of any one of clauses 13-23, wherein the one or more modulators are trifluoroacetic acid.
Clause 25. The method of any one clauses 13-24, wherein the one or more perfluoroalkyl or polyfluoroalkyl substances is perfluooctanoic acid (PFOA).
Clause 26. The method of any one of clauses 13-25, wherein the composition is noncovalently adhered or covalently attached to a surface.
Clause 27. The method of any one of clauses 13-26, wherein the surface comprises cellulose, silica, a polymer, a copolymer, a resin, a filter, or a combination thereof.
Clause 28. The method of any one of clauses 13-27, wherein the composition is dissolved in a solvent.
Clause 29. The method of any one of clauses 13-28, wherein the change is an increase in fluorescence intensity.
Clause 30. The method of any one of clauses 13-29, wherein the change in fluorescence intensity indicate removal of the perfluoroalkyl or polyfluoroalkyl substances from the sample.
Clause 31. The method of any one of clauses 13-30, wherein the composition has a limit of detection of about 1-50 ppb.
Clause 32. A method for detecting or removing perfluoroalkyl or polyfluoroalkyl substances in a sample, the method comprising:
   contacting the sample with a composition comprising at least one metal-organic framework (MOF), or salt thereof, having a plurality of metal-containing secondary building units (SBU) linked together by one or more bridging ligands,
      wherein said bridging ligands, or salts thereof, have a formula (II):

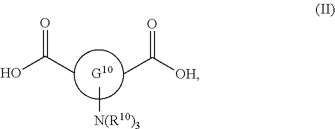

wherein
R¹⁰, at each occurrence, is independently hydrogen or C₁₋₈alkyl; and
G¹⁰ is independently a 6- to 12-membered aryl or a 5- to 12-membered heteroaryl.
detecting a change in fluorescence intensity.
Clause 33. The method of clause 32, wherein the bridging ligands have a formula (IIa):

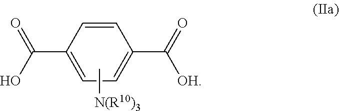

Clause 34. The method of clauses 32 or 33, wherein R¹⁰ at each occurrence is C₁alkyl.
Clause 35. The method of any one of clauses 32-34, wherein the bridging ligands, or salts thereof, are selected from:

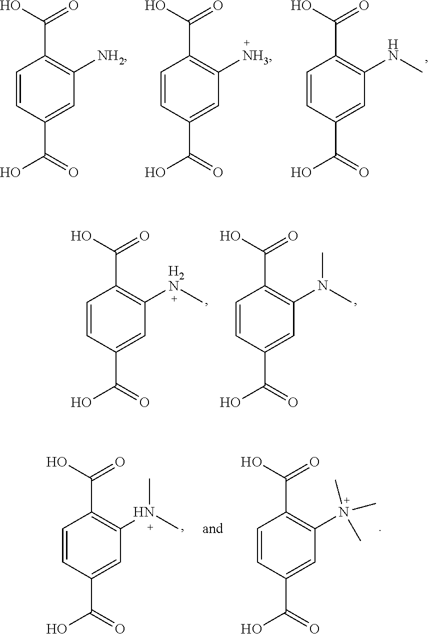

Clause 36. The method of any one of clauses 32-35, wherein the secondary building units comprise one or more metal atoms and one or more carboxylates.
Clause 37. The method of any one of clauses 32-36, wherein the metal atoms comprise zirconium(IV) or hafnium(IV).
Clause 38. The method of any one of clauses 32-37, wherein the one or more perfluoroalkyl or polyfluoroalkyl substances is perfluooctanoic acid (PFOA).
Clause 39. The method of any one of clauses 32-38, wherein the composition is noncovalently adhered or covalently attached to a surface.
Clause 40. The method of any one of clauses 32-39, wherein the surface comprises cellulose, silica, a polymer, a copolymer, a resin, a filter, or a combination thereof.
Clause 41. The method of any one of clauses 32-40, wherein the composition is dissolved in a solvent.
Clause 42. The method of any one of clauses 32-41, wherein the change is an increase in fluorescence intensity.
Clause 43. The method of any one of clauses 32-42, wherein the change in fluorescence intensity indicates removal of perfluoralkyl or polyfluoralkyl substances from the sample.
Clause 44. The method of clauses 32-43, wherein the composition has a limit of detection of about 1-50 ppb.

### EXAMPLES

### Example 1: PDI-Based MOFs for the Detection of PFAS

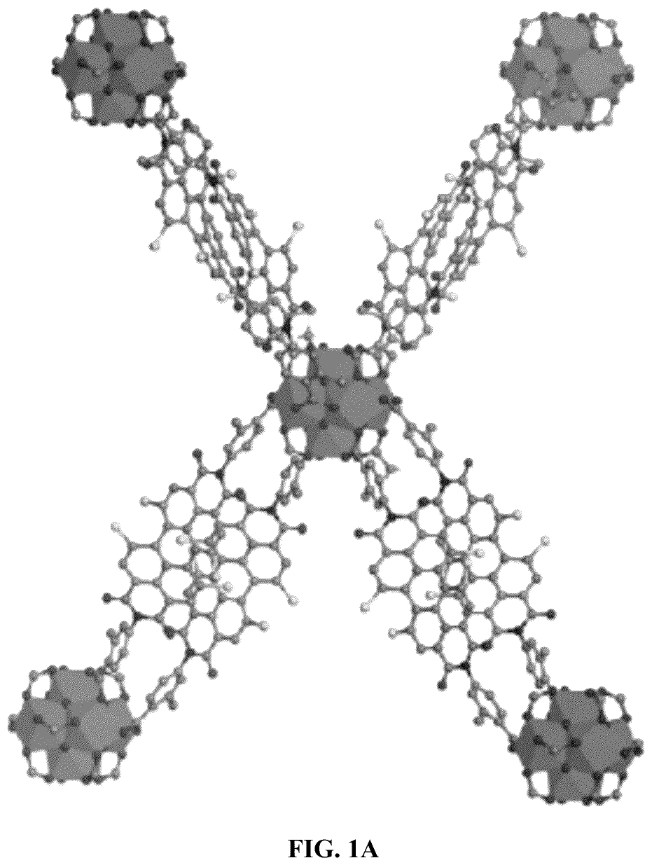

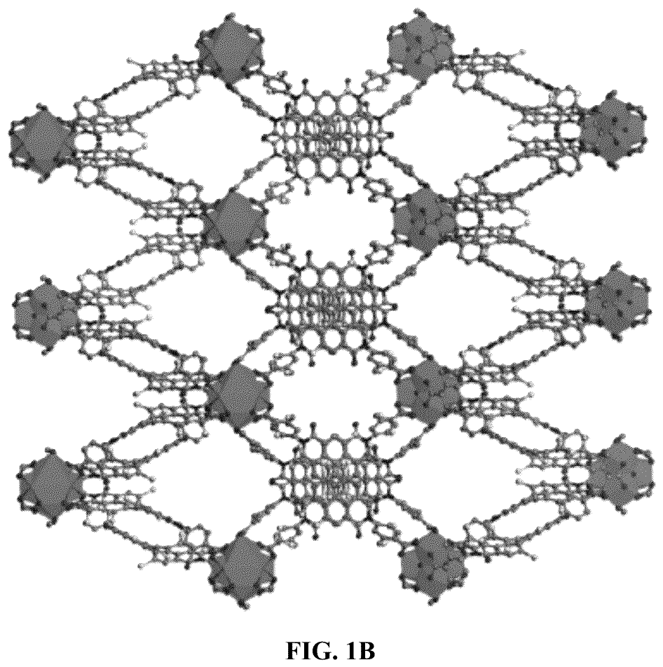

High adsorption capacity for perfluoroalkyl or polyfluoroalkyl substances (PFAS) is crucial for the dual functionality of MOF of detecting and removing PFAS. Described herein is the design and synthesis of a nonfluorinated, water-stable MOF with hydrophobic 3,4,9,10-Perylenetetracarboxylic diimide (PDI) as the bridging ligand (FIG. 1A-B). Numerous PDI molecules yield well-defined supramolecular nanostructures driven by self-assembly. The PDI molecules, with coordinating carboxyl or pyridyl functional groups, undergo a coordination-driven self-assembly with metal ions. Coordination driven self-assembly generally forms one-dimensional or two-dimensional morphologies, which inhibits porosity of coordination compounds. Very few PDI based, porous MOFs have been reported yet due to these drawbacks.

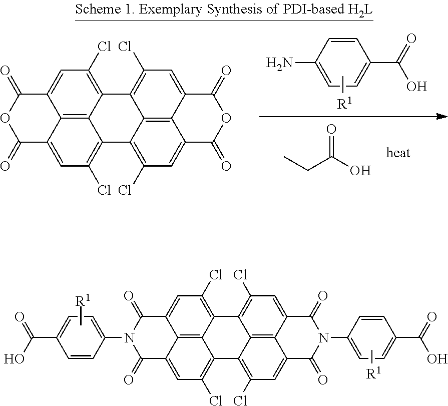

### Exemplary Synthesis

H₂L. 21,6,7,8-tetrachloroperylene-3,4,9,10-tetracarboxylic acid dianhydride (1.00 g, 1.89 mmol), 4-amino-2-methylbenzoic acid (2.85 g, 18.9 mmol), and propionic acid (25 mL) were stirred at 150° C. for 20 h. The resulting mixture was then allowed to cool to room temperature, followed by an addition of water to the mixture. The resulting precipitate was filtered, washed with a mixture of methanol and water (1:1), and dried under vacuum to obtain an orange solid. This solid was then subjected to recrystallization twice with DMF, resulting in the final desired product (orange powder). Yield: 1.06 g (70%).

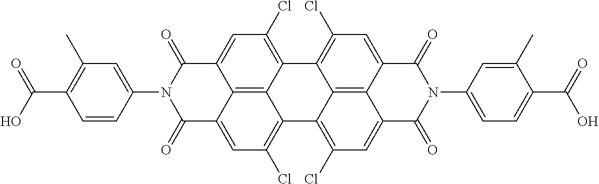 [SMILES: Cc1cc(N2C(=O)c3cc(Cl)c4c5c(Cl)cc6c7c(cc(Cl)c(c8c(Cl)cc(c3c48)C2=O)c75)C(=O)N(c2ccc(C(=O)O)c(C)c2)C6=O)ccc1C(=O)O]

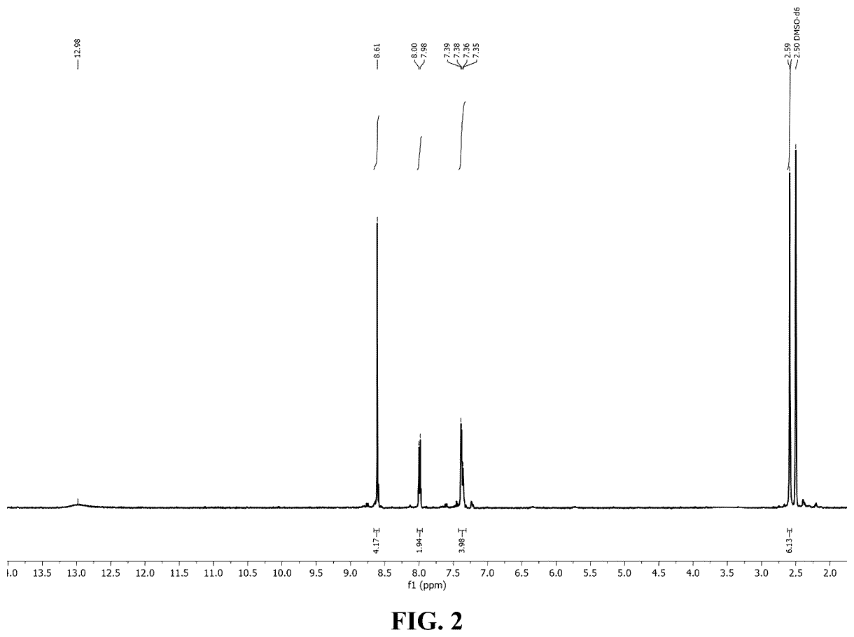

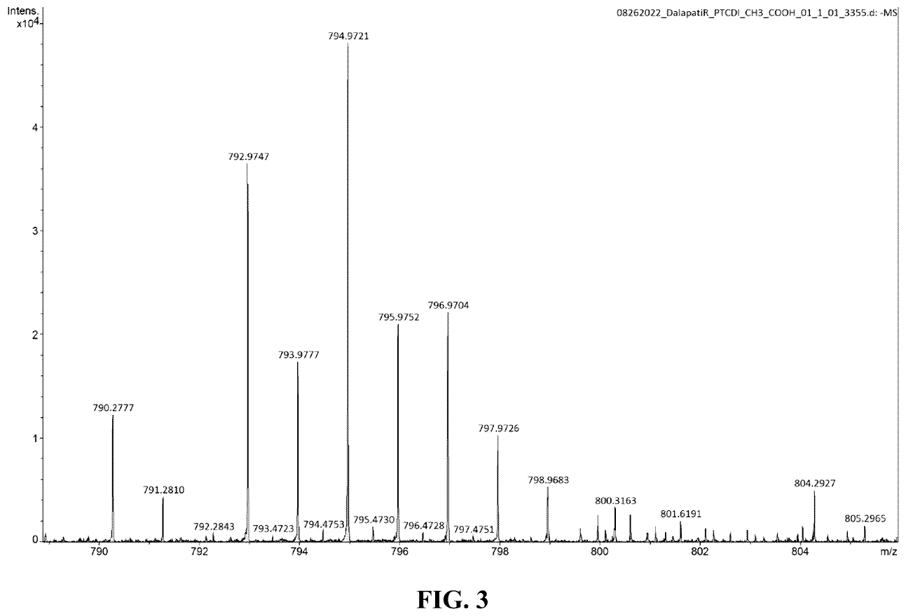

¹H NMR (400 MHz, DMSO) δ 8.61 (s, 4H), 7.98 (d, 2H), 7.35 (m, 4H), 2.59 (s, 6H). ESI-MS (m/z): [M−H⁺] Calcd. 793.98, found 792.97. The NMR and mass spectra of H₂L ligand are shown in FIG. 2 and FIG. 3.

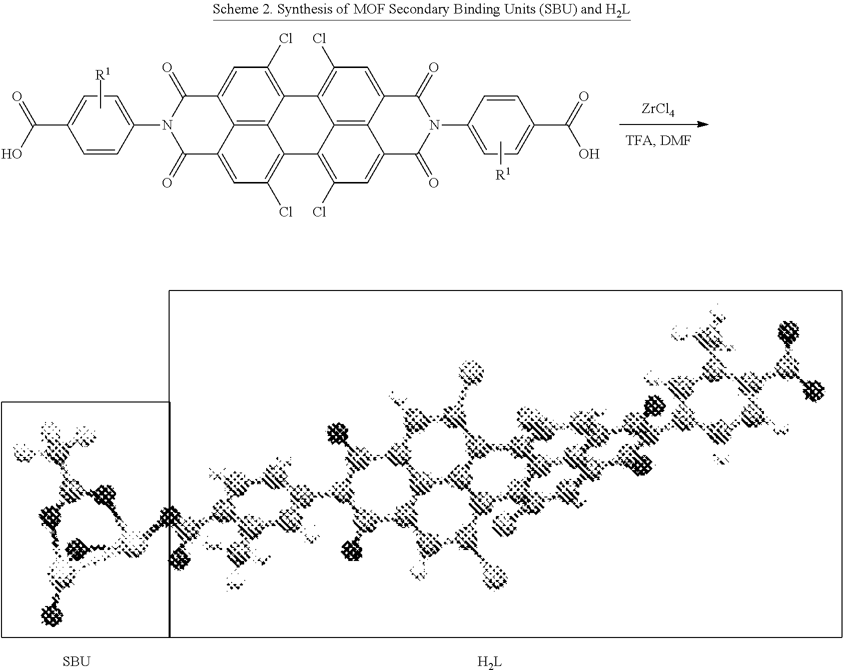

U-1. A mixture of ZrCl₄ (29 mg, 0.125 mmol), H₂L ligand (50 mg, 0.0625 mmol) and trifluoroacetic acid (TFA) were dissolved in 15 mL of N,N-dimethylformamide (DMIF) by sonication for 10 min. The resulting dark red solution was transferred into a teflon liner and heated at 100° C. for 24 h. After being cooled to room temperature, the red powder was collected by filtration and washed with large volumes of DMF and acetone. After filtration, the MOF sample was dried in an oven.

### General Experimental Material and Methods

Physical Measurements. Fourier transform infrared (FT-IR) spectra were collected in the region of 440-4000 cm-1 with a Nicolet iS50 FT-IR spectrometer. Thermogravimetric analyses (TGA) were carried out with a TA Instruments Discovery SDT 650 thermogravimetric analyzer in a temperature range of 30-700° C. in an air atmosphere. Ambient-temperature powder X-ray diffraction (PXRD) patterns were measured on a Bruker D2 Phaser X-ray diffractometer operated at 30 kV and 10 mA using Cu Kα (λ=1.5406 Å) radiation. The nitrogen sorption isotherms up to 1 bar were recorded using a Micromeritics 3Flex gas sorption analyzer at −196° C. Before the sorption measurement, the compound was degassed at 80° C. for 24 h under dynamic vacuum. Steady-state fluorescence studies were carried out by using an Agilent Cary Eclipse fluorescence spectrophotometer. Fluorescence images were taken with a Leica DMi8 fluorescent microscope. NMR data were recorded on a Varian Mercury 400 MHz spectrometer. X-ray photoelectron spectroscopy (XPS) measurements were performed using a Kratos Axis Ultra DLD x-ray spectrometer. Thermo Scientific Advantage Data System software was used for data analysis with the C 1s reference peak at 284.8 eV. A Bruker maXis II ETD Q-TOF LC/MS instrument was used to obtain high resolution ESI MS data. Scanning electron microscopy (SEM) images were obtained on an FEI Nova NanoSEM™ scanning electron microscope.

Filter paper-based PFOA detection protocol. The U-1 MOF was coated on Whatman filter paper (˜1 cm×1 cm) by immersing the paper in MOF suspension made in ethanol, followed by oven drying. The process was repeated three times to increase MOF loading on the paper strip. The MOF loading amount was ˜5 mg/cm2 (measured by taking weight before and after loading). L of various low concentration (10-50 nM) solutions of PFOA were added to paper strips (˜1 cm×1 cm) and solvents subsequently dried. The process was repeated five times (total volume added 75 μL) to ensure adequate accumulation of PFOA. Finally, 10 μL DMF was added to the PFOA accumulated paper and emission intensity was measured.

Digestion protocol of the MOF sample for recording 19F NMR spectrum. 10 mg of MOF sample was added to 0.5 mL of DMSO-d6. To this solution, 100 μL of saturated solution of tripotassium phosphate in D₂O was added. After incubating for 30 min at 70° C., the MOF sample became dissolved and the organic phase was filtered. Clear red colored solution was analyzed by ¹⁹F NMR spectroscopy.

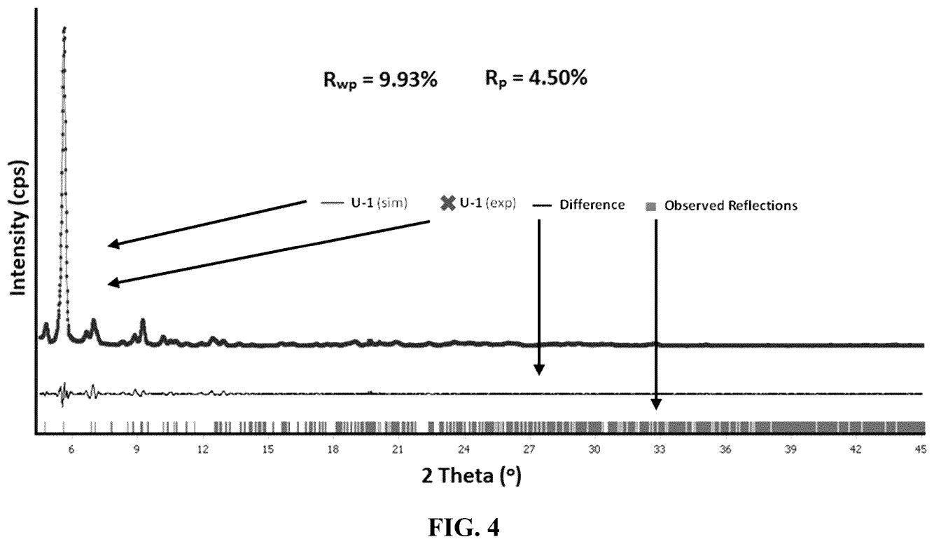

Pawley refinement. The powder X-ray diffraction (PXRD) pattern of U-1 was measured in a reflection geometry using CuKα1 radiation and indexed with the help of STOE WinXPow software (Found: I-centered cell, a=b=18.885(13) Å and c=83.056(22) Å with a space group, I4₁/a). In U-1, the model of the methyl functionalized PTCDI ligand molecule was similar to the ligand molecule used previously by Lü et al. to make Zr-PDI MOF. This structural simulation and refinement are necessary since the ligand molecule in U-1 has a methyl-functional group and trifluoroacetic acid (TFA) was used as a modulator. The simulated structure was obtained by imposing the structure of Zr-PDI, converting the ligand molecule, and subsequently optimized by force-field calculation using the universal force field as implemented in the Materials Studio software. An attempted full Pawley refinement of this model was not possible, which may be due to the length of the ligand molecule, the large unit cell and the low number of reflections above 20°. Some relevant cell parameters for the modelling are summarized in Table 1 and the final plot is shown in FIG. 4. The simulated PXRD data correlate suitably with the experimental data), although the peak intensities deviate for the reasons stated above.

TABLE 1
The unit cell parameters of U-1 compared to Zr-PDI MOF.

| Compound name | Zr-PDI¹ | U-1 |
|---|---|---|
| Space group | I 41/a | I 41/a |
| Crystal system | Tetragonal | Tetragonal |
| a = b ≠ c (Å) | a = b = 18.085(3), | a = b = |
| | and c = 86.656(12) | 19.2570(6), |
| | | and c = 83.1554(7) |
| Volume (Å³) | 28343(9) | 30836.6874(20) |
| R_{wp} (%) | — | 9.93 |
| Rₚ (%) | — | 4.50 |

Sensing experiments for PFAS. The suspension of activated U-1 MOF was prepared by dispersing 6 mg of MOF powder in 3 mL of DMF. The mixtures were sonicated for 30 min. to create homogeneity throughout the dispersion before fluorescence measurements were taken. 0.2 mL of the as-prepared stock solution were mixed with 2.8 mL of water/DMF (60/40 v/v) in a quartz cuvette (final MOF concentration: 0.13 mg/mL). The emission spectra of the U-1 MOF suspension were obtained at an excitation wavelength (λₑₓ) of 490 nm. The analytes and interferents used for the sensor testing experiments were PFOA, PFOS, octanoic acid, lauric acid, trifluoracetic acid (TFA), and cetyltrimethyl ammonium bromide (CTAB), for which 10 mM stock solutions were prepared in water. Selected PFAS and other interferents were added gradually into the U-1 suspension, and the mixture was thoroughly mixed before recording the fluorescence spectra. The spectra were recorded three times for each sample and the averaged data were plotted to ensure accuracy and reliability.

Filter paper based PFOA detection. The U-1 MOF was coated on Whatman filter paper (˜1 cm×1 cm) by immersing the paper in MOF suspension made in ethanol, followed by oven drying. The process was repeated three times to increase MOF loading on the paper strip. The MOF loading amount was ˜5 mg/cm2 (measured by taking weight before and after loading). 15 L of various low concentration (10-50 nM) solutions of PFOA were added to paper strips (˜1 cm×1 cm) and solvents were subsequently dried. The process was repeated five times (total volume added 75 μL) to ensure adequate accumulation of PFOA. Finally, 10 μL DMF was added to the PFOA accumulated paper and emission intensity was measured.’

Regeneration of MOF. After each consecutive sensing cycle, the MOF suspension was carefully transferred into a centrifuge tube. The solution was then subjected to centrifugation for 10 minutes at 4000 rpm. The supernatant solution was decanted, and 10 mL of a 30/70 v/v mixture of 0.1 M HCl and methanol was added. This mixture was sonicated for 30 minutes before being centrifuged at 4000 rpm for 10 minutes to collect the MOF particles. This process was repeated three times. The MOF was then washed twice with deionized water followed by acetone and collected via centrifugation. The regenerated MOF was dried in a vacuum oven at 60° C. before the next sensing experiment.

Quantum Yield Calculation. The fluorescence quantum yield of the U-1 MOF, before and after PFOA sensing, was evaluated by Parker-Rees method using Rhodamine 6G as a standard fluorophore. The Parker-Rees equation can be written as follows:

$${Φ}_{u} = ( {A}_{s} {F}_{u} {n}_{u}^{2} / {A}_{u} {F}_{s} {n}_{s}^{2} ) {Φ}_{s} \qquad ( 1 )$$

Where (s represents the quantum yield of the reference compound (Rhodamine 6G, with a value of 0.95) and (Dᵤ denotes the quantum yield of the MOF sample. As and Aᵤ stand for the absorbances of Rhodamine 6G and the MOF sample at the excitation wavelength (490 nm), respectively. To mitigate the reabsorption of fluorescence light passing through the samples, their absorbance maxima were maintained below 0.1. Fₛ and Fᵤ denote the integrated fluorescence intensity areas of Rhodamine 6G and the MOF sample when excited at the same wavelength, respectively. The refractive indices of the solvents for the MOF sample suspension and Rhodamine 6G are denoted by nᵤ and nₛ respectively.

### Synthesis and Activation of U-1 Metal-Organic Framework (MOF)

Initially, the synthetic procedure reported by Lü et al. (Lü, B., et al. Nat. Commun. 2019, 10 (1), 1-8) was used. In the Lü et al publication acetic acid as a modulator was used to prepare a perylene diimide based MOF (Zr-PDI). Unfortunately, no crystalline MOF material was obtained for the PDI-based ligand (H₂L) using the Lü et al. procedure. As a result, alternative synthesis conditions were explored using different zirconium salts (ZrCl₄ and ZrOCl₂·8H₂O) and dimethylformamide (DMF) as a solvent. Different temperatures and reactions times were evaluated. Various modulators, such as benzoic acid, acetic acid, formic acid, proline, and trifluoroacetic acid were also tested. Parameter testing was performed to increase the crystallinity of the MOFs (Table 2).

TABLE 2
Modulators examined in the presence of different metal crystals

| | Benzoic | Acetic | Formic | Trifluoroacetic | |
|---|---|---|---|---|---|
| Modulator | Acid | Acid | Acid | Acid | Proline |
| ZrCl₄ | X | X | X | ✓ | x |
| ZrOCl₂ 8H_{2O} | X | X | X | X | X |

Optimized synthesis conditions were achieved by using ZrCl₄ as the metal salt, trifluoroacetic acid as the modulator, and DMF as the solvent. Crystalline powder of U-1 MOF was obtained by heating the reaction mixture for 24 hours at 100° C. The significant difference in synthesis protocol between U-1 and the previous Zr-PDI MOF is the methyl modification at the benzoic group of the PDI ligand, which changes the local hydrophobicity around the metal cluster.

### MOF Structural Analysis

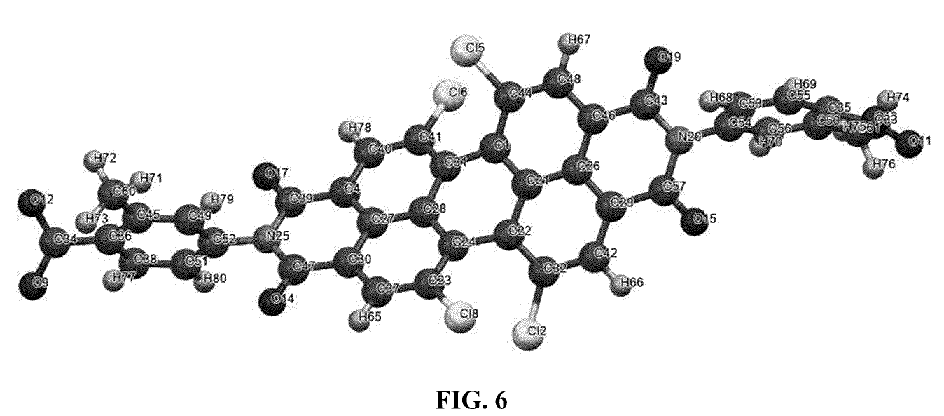

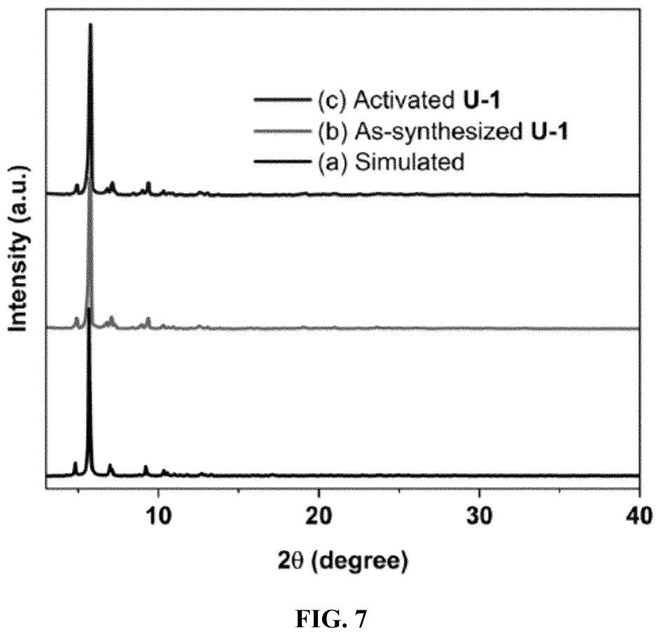

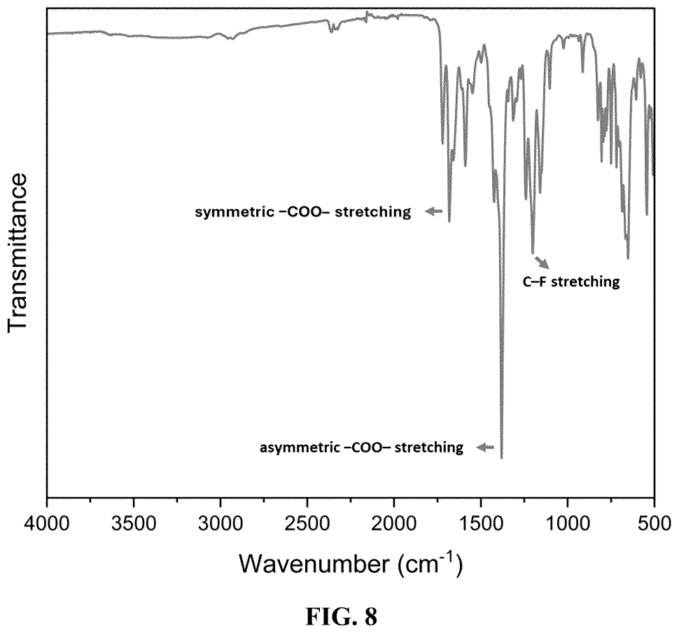

The solvothermal reaction between H₂L (FIG. 1A) and ZrCl₄ in DMF at 100° C. with trifluoroacetic acid as a modulator produced the U-1 MOF. The XRPD patterns of U-1 were indexed in tetragonal crystal system with the space group I41/a with a=b=19.2570(6), and c=83.1554(7) Å. The Pawley refinement data indicates that U-1 is isostructural with the Zr-PDI MOF recently reported by Lü et al. for which a tetrachloro-substituted PDI ligand was prepared without the methyl modification at the benzoic group. The six vertices of the Zr₆(μ₃-O)₄(μ₃-OH)₄ octahedral cluster are occupied by Zr(IV) centers and the eight triangular faces are alternatively capped by four μ₃-OH and four μ₃-O groups. The [Zr₆(μ₃-O)₄(μ₃-OH)₄]¹²⁺ SBU is coordinated by twelve carboxylate units, eight H₂L ligands and four TFA molecules. The asymmetric unit of U-1 contains two crystallographically independent Zr atoms, one L²⁻ ligand molecule, one TFA molecule and two μ₃-O atoms (FIG. S5). Due to the electrostatic repulsion and steric hindrance arising from the tetrachloro and methyl substitutions, the central conjugated ring of H₂L ligand is highly twisted with a dihedral angle ∠(C—C—C—C) of 36.9° (FIG. 6). This twisted structure of the H₂L ligand in crystalline U-1 MOF highlights the axial chirality of the PDI cored ligand. As depicted in FIG. 1A-1B, the Zr₆(μ₃-O)₄(μ₃-OH)₄ SBUs are connected to each other via the L²-ligands, which forms a 3D polymeric structure due to chirality differences. The results of Pawley refinement confirmed that the simulated and experimental XRPD data are identical to each other for U-1 (FIG. 7).

### Characterization of U-1

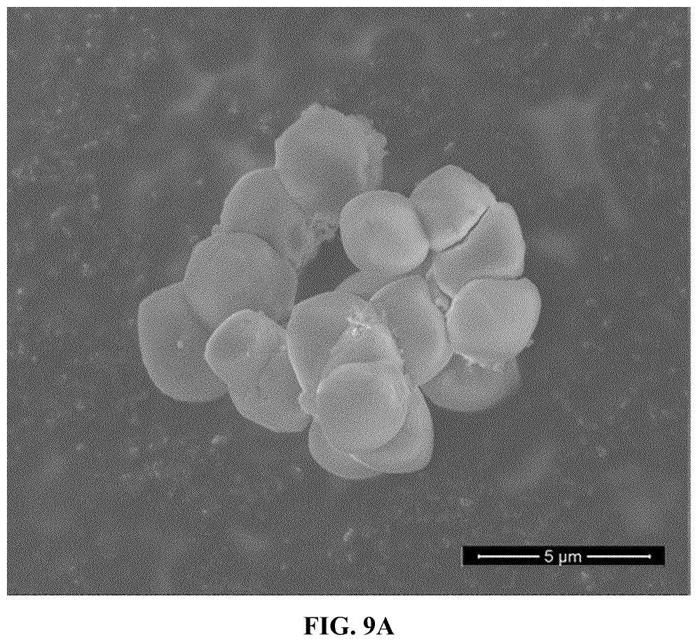

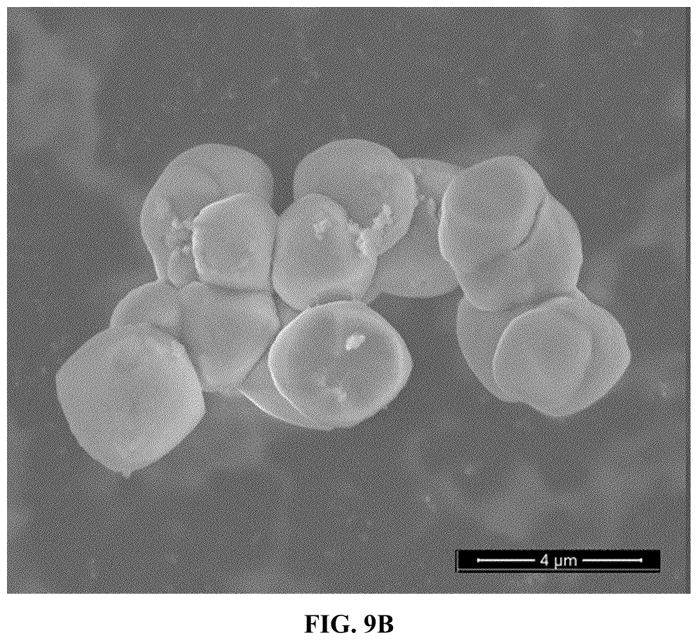

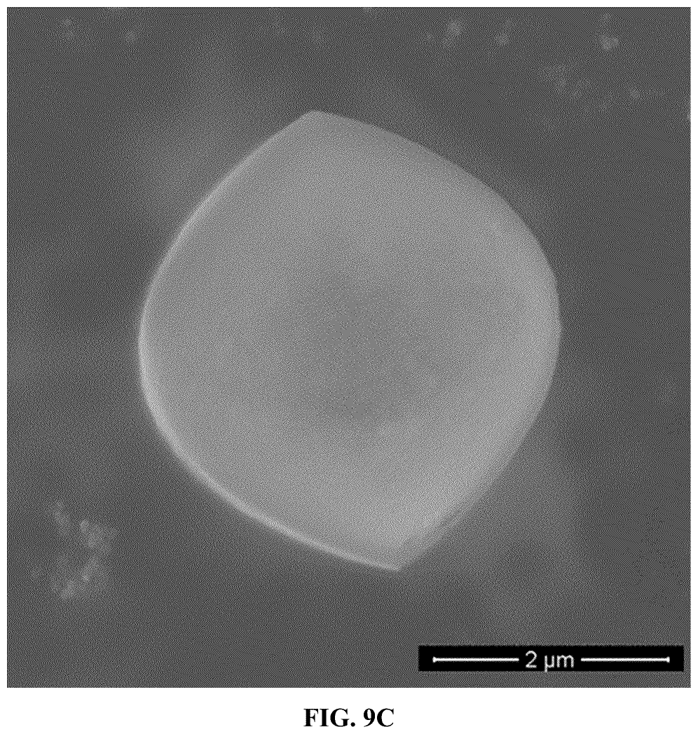

The phase purity of U-1 was confirmed by the XRPD measurement. The as-synthesized MOF material showed a good semblance with the simulated XRPD pattern (FIG. 7). The XRPD pattern of activated U-1 was also well-matched with the simulated XRPD pattern. This suggests that the guest-free form of U-1 has the same structural framework as the parent sample, implying the mechanical robustness of the framework during activation process. The high intensity peak in ATR-IR spectra around 1680 and 1383 cm⁻¹ for U-1 arises from the asymmetric and symmetric —COO— stretching vibrations, respectively, caused by the metal coordinated L² ligands (FIG. S8). The peak near 1201 cm-1, corresponding to C—F stretching, confirms the presence of a TFA molecule in the framework (FIG. S8). The SEM images (FIG. 9A-C) reveal a uniform distribution of micron-sized MOF particles with minimal presence of amorphous materials, which provide additional confirmation of the material's phase purity.

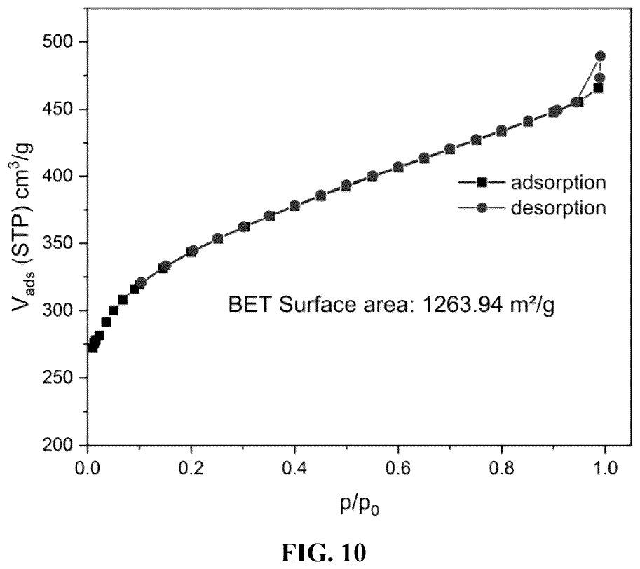

MOFs constructed with large ligand molecules tend to undergo pore collapse upon solvent removal. Permanent porosity is a crucial property for current MOFs constructed with PDI ligands (length >24 Å). N₂ sorption experiments were conducted to check the permanent porosity of the activated U-1 MOF. The N₂ sorption isotherms of U-1 are in agreement with IUPAC classifications, following a type I behavior (FIG. 10). From the N₂ adsorption isotherm, the specific BET surface area of U-1 was calculated to be 1263 m²g⁻¹. This again may indicate that the U-1 MOF can retain its porosity during the activation process.

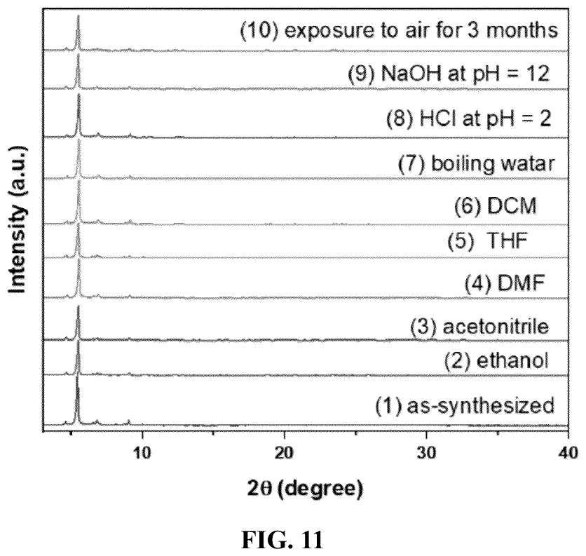

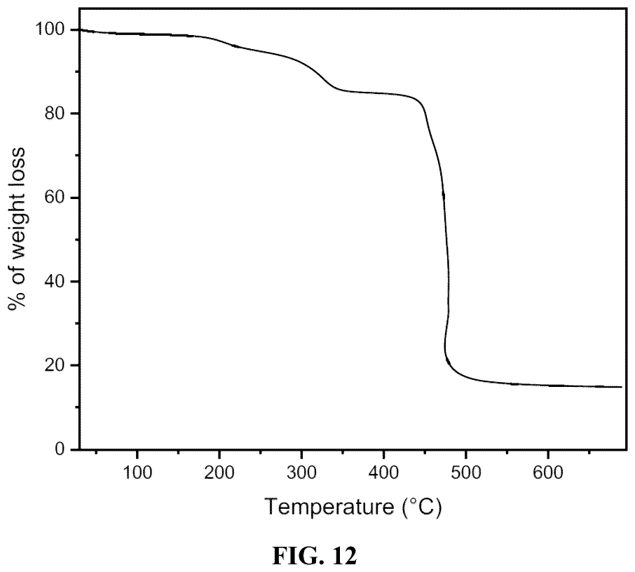

To verify the chemical stability of U-1 MOF, the powder material was immersed in different kinds of liquids (e.g., ethanol (EtOH), acetonitrile, tetrahydrofuran (THF), and dimethylformamide (DMF) etc.) overnight. The MOF material was then recovered by centrifugation, and the XRPD patterns were recorded. The XRPD patterns of the solvent treated U-1 remained almost unchanged compared to the untreated sample (FIG. 11). These results suggest that the framework of U-1 was unaffected when dispersed in various liquids. For PFAS detection applications, the U-1 must survive different pH media in industrial wastewater or environmental water. To confirm the framework stability of U-1 in varying pH environments, the MOF powder was treated with HCl (pH=2) and NaOH (pH=10) for 12 hours. The XRPD patterns were then acquired. As depicted in FIG. 11, the XRPD patterns remain almost unchanged upon treatment in acid or base, further affirming the strong chemical stability, which is conducive to sensor application in broad range of water samples. In addition to chemical stability, U-1 also shows thermal stability up to 400° C., as confirmed by thermogravimetric analyses (FIG. 12). Thermal and chemical stability of U-1 prove the relevance of the material for use in real-field chemical detection.

### Photophysical Behavior of MOF

PDIs have received considerable attention as fluorophores due to their high photochemical and thermal stability in ambient conditions. Based on the high structural tunability at the bay- and imide-positions, numerous molecules of PDI-based materials have been developed as chemical sensors. The PDI based fluorophore shows interesting emissive behavior in a fully dissolved state in solution. A strong tendency towards intermolecular π-π stacking of PDI molecules leads to aggregation-caused quenching (ACQ), which decreases the emission in an aggregated or solid state. Such ACQ phenomenon restricts the application of PDIs as a solid-state sensor material. Prevention of close π-π stacking in solid state PDI is challenging. In metal organic frameworks, the ligand molecules can self-assemble via metal coordination in a well-defined manner in the crystal system. This is unique and not applicable to other types of self-assembly processes. The formation of PDI ligand-based MOF could be beneficial to mitigate the ACQ effect on PDI fluorophores.

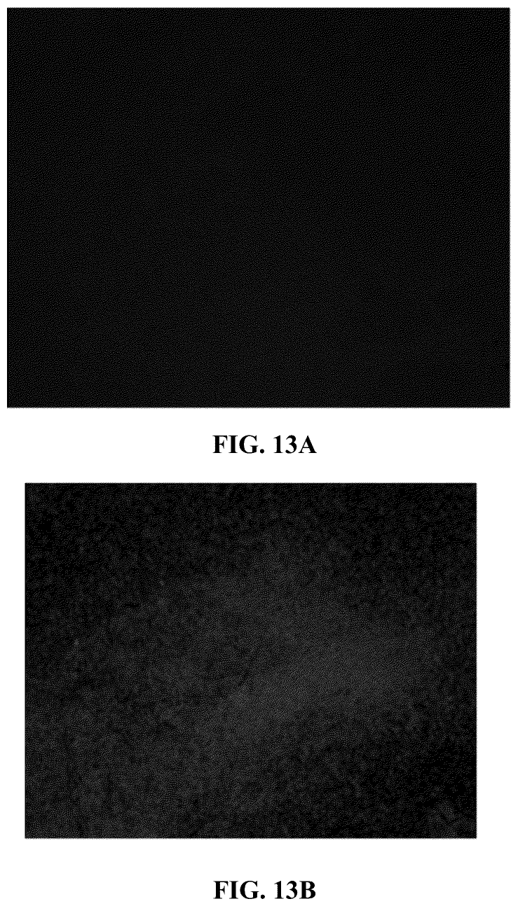

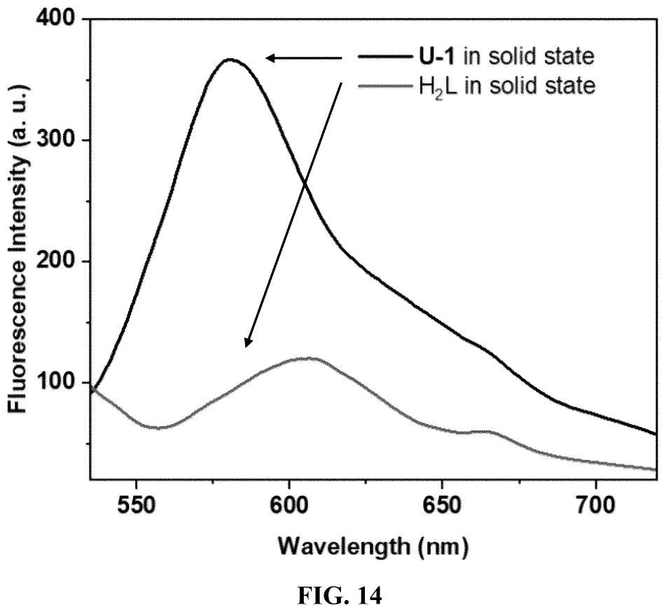

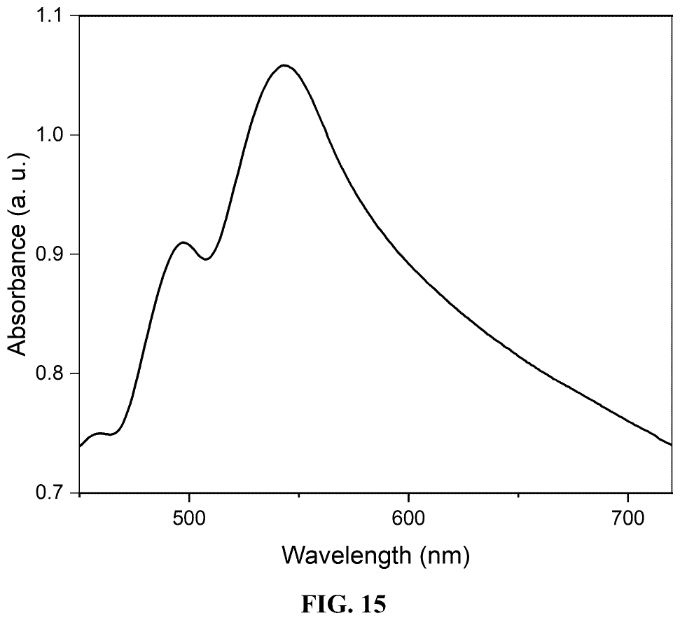

To verify this hypothesis, fluorescence microscopy measurements were performed on the free ligand H₂L and U-1 MOF deposited on a glass substrate (FIG. 13A and FIG. 13B). U-1 shows much stronger red emission compared to the free ligand under the same imaging condition. This observation is consistent with the fluorescence spectral measurement performed on the same samples (FIG. 14), for which the U-1 MOF exhibits broad emission band with maximum at 580 nm, while the aggregate of H₂L ligand shows much weaker emission with maximum wavelength shifted to ca. 605 nm. Red shift of emission band was indicative of the aggregation of PDI molecules. Apparently, the blue shift of emission maximum and increase in intensity observed for the U-1 MOF is caused by the coordination of the PDI ligand to Zr(IV), which in turn weakens the π-π stacking of PDI backbones, thus mitigating the ACQ effect. The presence of close shell d⁰ electronic configuration of the Zr⁴⁺ metal ion also excludes any possibility for paramagnetic fluorescence quenching of the ligand molecules.

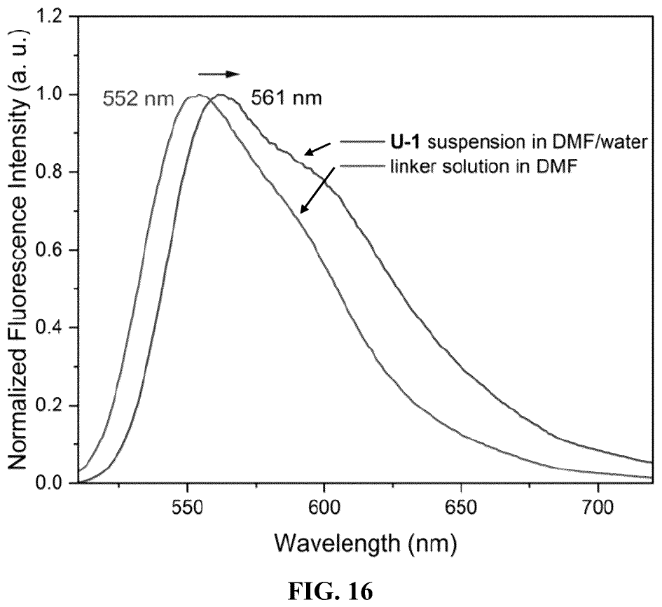

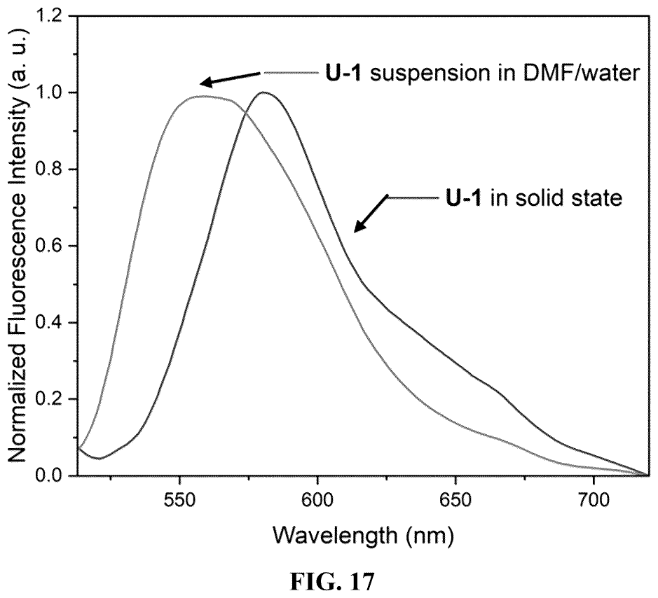

The UV-Vis spectrum of U-1 MOF shows two strong absorption bands near 543 nm and 496 nm, with a broad shoulder peak around 456 nm (FIG. S15). This characteristic absorbance of U-1 MOF could be assigned to the 0-0, 0-1, and 0-2 transition energies of the PDI molecule. When compared to the molecularly dispersed state of H₂L ligand in solution phase with emission maximized at 552 nm (FIG. 16), the fluorescence spectrum measured for the U-1 MOF demonstrates a ˜4 nm red shift. This implies a conformational change of the p-conjugation structure of PDI backbone, particularly the dihedral twisting angle around the four chloro-moieties. Additionally, when compared to the solid state and dispersed state emission of U-1 in binary water/DMF mixture, a significant blue shift was observed in dispersed state (FIG. 17). This may be attributed to the structurally relaxed state achieved through polar solvation processes in the high dielectric environment, as opposed to the solid state.

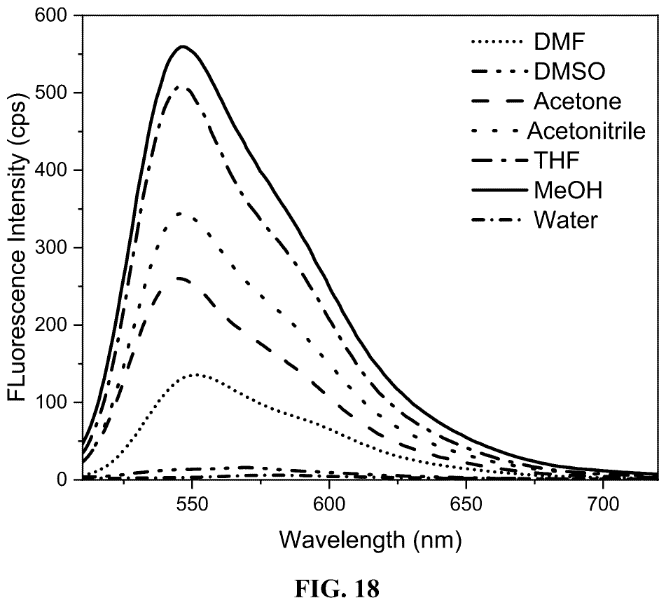

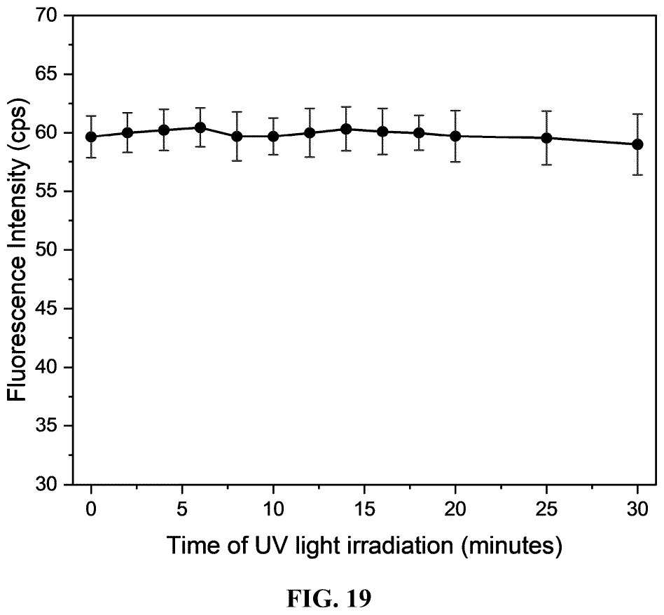

The interesting fluorescence behavior of the current MOF in both solid and dispersed states provided motivation to investigate the impact of solvents on the MOF's emission. The fluorescence emission intensity of the MOF is notably reduced in water, the most polar solvent among those tested, as shown in FIG. 18. On the contrary, solvents with lower polarity, such as methanol and tetrahydrofuran, exhibit higher fluorescence levels. This trend may be attributed to the pronounced dipole-dipole interactions between the twisted perylene core of the MOF and the solvent molecules, particularly evident in highly polar solvents. Consequently, solvents with elevated polarity effectively inhibit the radiative relaxation of the perylene core through strong interactions, leading to a decrease in the MOF's fluorescence. The MOF demonstrates its lowest fluorescence intensity in water, owing to its highest polarity among the selected solvents. The photostability of a potential sensor material remains a critical factor considered for real-world applications. Particularly, some PDI based materials may not be photostable under continuous irradiation. The MOF suspension was continuously irradiated with UV light (λ=365 nm, 6 W, provided by UVGL-58 handheld UV Lamp) and emission maxima were monitored in time intervals. As depicted in FIG. 19, almost no change in emission intensity was observed for U-1 MOF even after 30 minutes of UV light irradiation. This confirms the photostability of the current, developed MOF. These results enabled us to develop the U-1 MOF as a fluorescence turn-on sensor for chemicals like PFOA (vide infra) that may compete for binding with the same metal center, resulting in turning on the fluorescence emission of PDIs.

### Detection of PFOA

The photophysical properties and porous nature of PDI-based MOF descried herein led to the investigation of response of the MOF towards PFOA. The Zr-PDI MOF, recently reported, does not possess the methyl groups at the phenyl moiety. These pre-functionalized hydrophobic groups are especially critical for enhancing the binding and detection of hydrophobic target analytes, like PFAS compounds, leveraging the strong hydrophobic interactions. The use of a methylated ligand also helps maintain hydrophobic pore walls of MOF, thus enhancing water stability, and helping preserve the porosity that is crucial for the internal diffusion of PFAS. Additionally, the bridge ligand being a non-fluorinated ligand makes the MOFs described herein less toxic compared to other hydrophobic, water-stable MOFs.

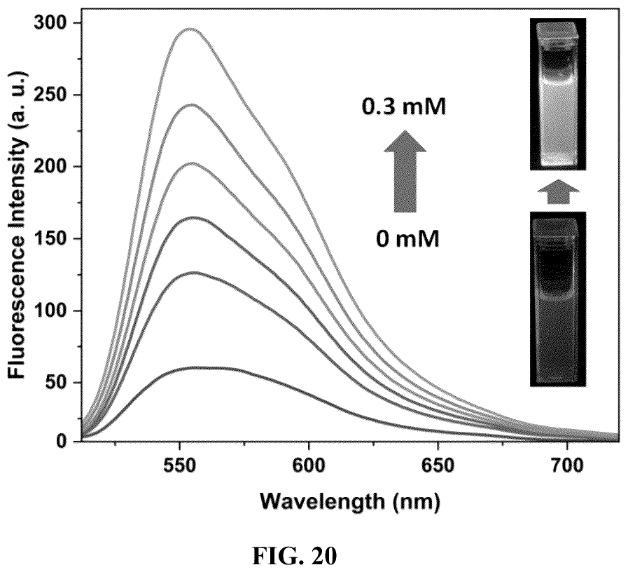

The fluorescence spectra of the dispersed MOF powder in water/DMF (60/40: v/v) was recorded with incremental additions of aqueous PFOA solution (FIG. 20). A rapid turn-on response was observed with addition of PFOA to the MOF suspension. The visible reddish-yellow emission was observed upon increase in the concentration of PFOA (inset of FIG. 20), implying the feasibility of quick detection of PFOA even by naked-eye visualization of the emission formation.

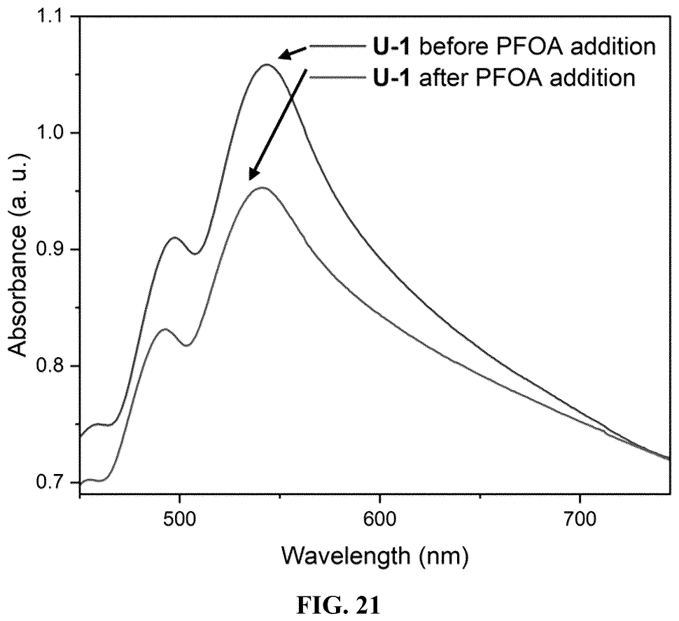

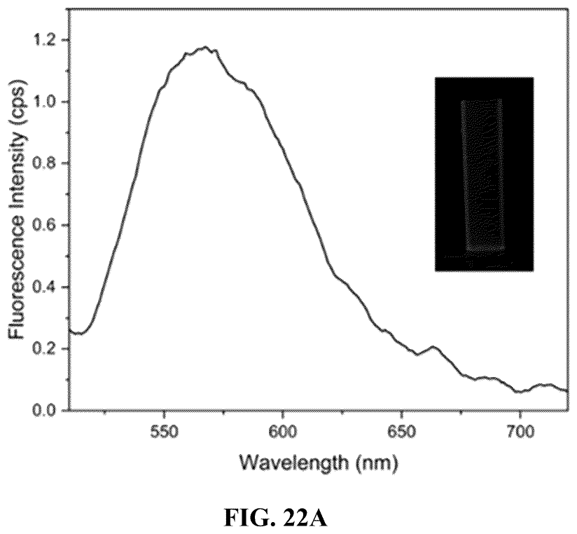

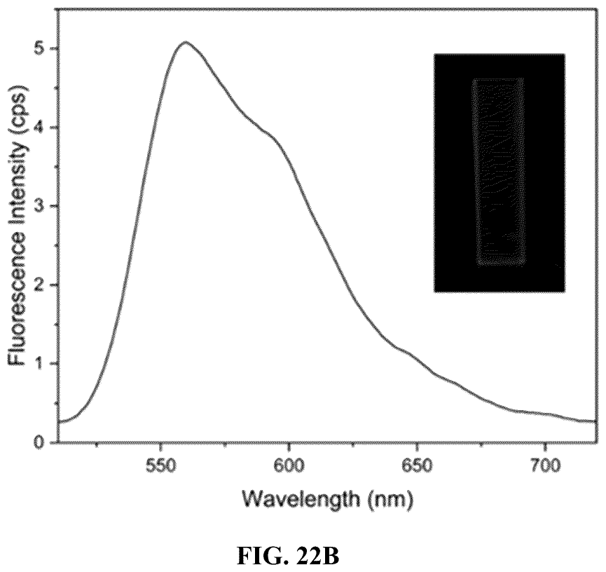

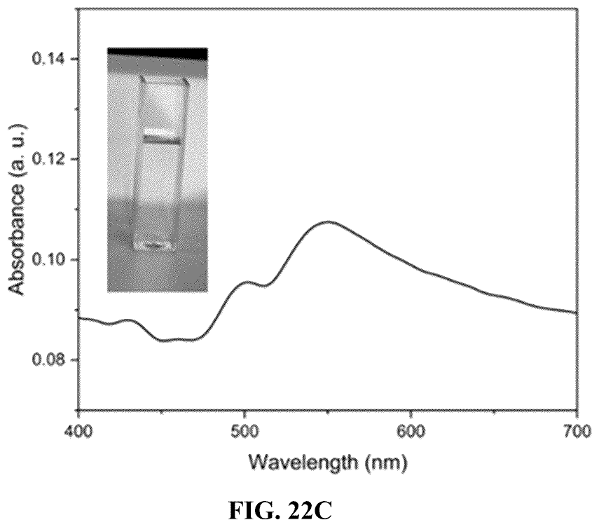

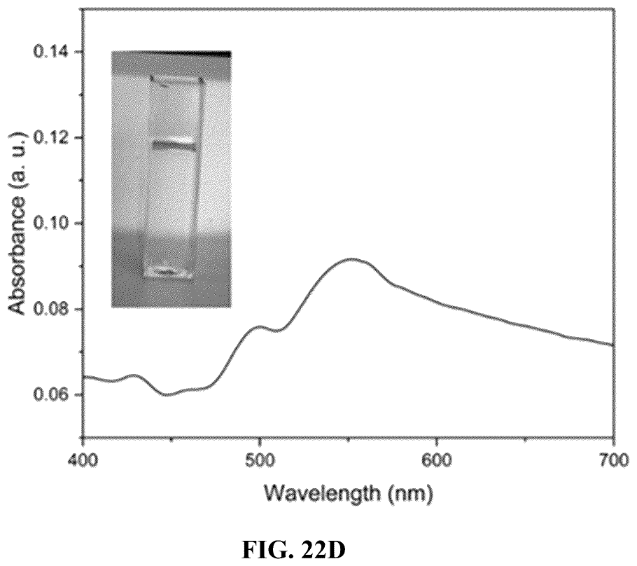

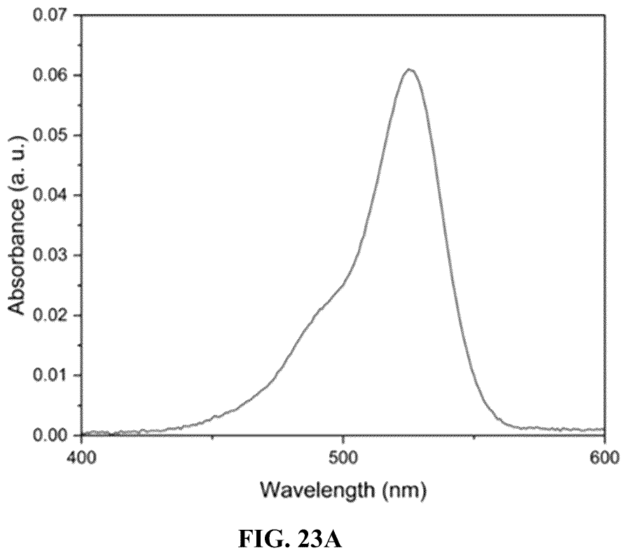

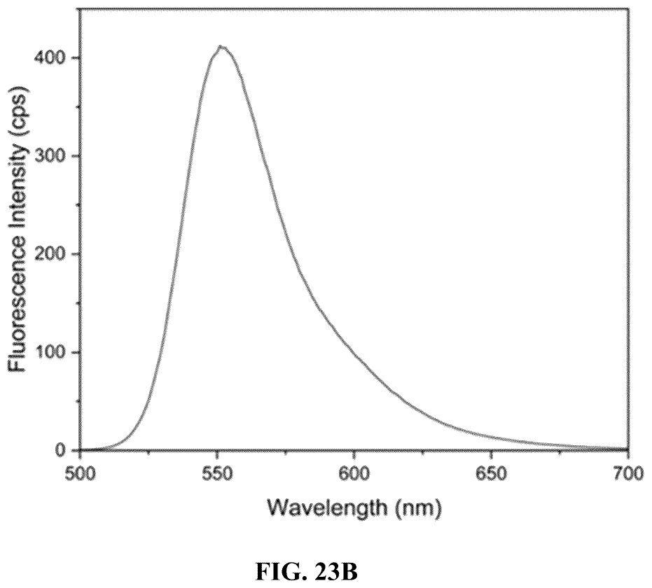

The UV-Vis spectrum of the MOF suspension was recorded post-PFOA sensing experiment and compared with the spectrum of the MOF suspension prior to PFOA addition. After PFOA sensing, the UV-Vis spectra of U-1 MOF retain about the same shape with the two prominent peaks around 543 and 496 nm slightly blue shifted to 540 and 492 nm, respectively (FIG. 21). In our sensing experiments, 490 nm (close to one of the two absorption maxima) was chosen as the excitation wavelength to monitor the turn-on emission in response to PFOA binding. The slight blue shift observed in the absorption spectra can be attributed to structural changes in the MOF induced by PFOA binding, as elaborated in the mechanism section. The fluorescence quantum yield of the system also increased from 0.0 to 0.02 after PFOA addition (FIG. 22A-D and FIG. 23A-B).

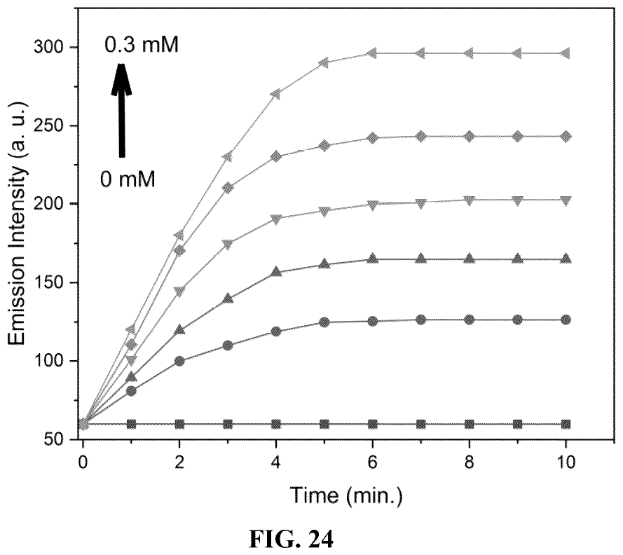

To assess the response time of U-1 MOF for detecting PFOA, time-dependent experiments were carried out with various concentrations of PFOA solutions (FIG. 24). For each concentration of PFOA, the emission intensity (at λₘₐₓ=561 nm) of U-1 MOF suspension was measured as a function of time after addition of PFOA. The results demonstrate that the U-1 MOF exhibits a rapid response, taking less than 5 minutes to reach adsorption equilibrium with PFOA. The sensing response time observed may be relatively longer when compared to other MOF based sensors used for detection of small ions or molecules. The relatively long response may be attributed to the slow diffusion of PFOA within the nano-pores of MOF. The adsorption equilibrium of PFOA is determined by the complexation of the carboxylic head group of PFOA with the Zr(IV) ion in conjunction with the hydrophobic interaction between the fluorinated alkyl chain of PFOA and the chlorinated backbone of PDI. While the mesoporosity of MOF enhances the adsorption capacity, the spatial confinement may also limit the diffusion kinetics. This represents a typical trade-off in materials design and optimization.

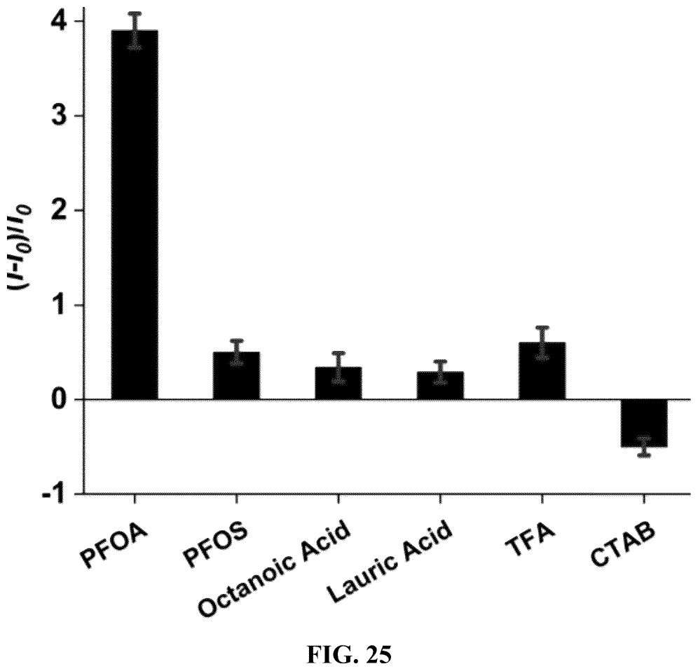

To examine the selectivity of U-1 towards PFOA, another common PFAS compound, PFOS was also tested under the same conditions. Surprisingly, only a slight turn-on response was observed for PFOS (FIG. 25). This is likely due to the relatively weaker complexation of sulfonic group (compared to the carboxylic group of PFOA) with the Zr(IV) ion. Indeed, while ligands with carboxylic groups are common for synthesis of stable MOFs, it is rare to have a MOF synthesized from sulfonic based ligands. Trifluoro acetic acid (TFA), a short chain analogue of PFOA, was also tested and demonstrated a much lower response under the same conditions compared to PFOA (FIG. 25). This indicates that the long alkyl chain of PFOA plays a crucial role in the strong binding interaction with MOF, for which the longer chains exhibit stronger hydrophobic interactions with the PDI ligand. In addition to fluorinated compounds, other long chain carboxylic acids (octanoic acid and lauric acid) and common surfactants such as cetyltrimethyl ammonium bromide (CTAB), were also examined for the same sensing response. These compounds have a similar alkyl structure to PFOA, but without fluorination. Interestingly, no significant turn-on response was observed from octanoic acid or lauric acid. This is likely due to the lower hydrophobicity of nonfluorinated alkyl chains, which in turn reduces the binding interaction with PDI ligands in MOF. More remarkably, the addition of CTAB did not result in any turn-on response. Rather, it caused a slight decrease in emission intensity (FIG. 25). This is not surprising when taking the positively charged head group of CTAB into account, which would not compete for the complexation with the metal center. Additional testing was also carried out using other fluorinated substances, namely pentafluorobenzoic acid (PFBA), 1H,1H,2H,2H-perfluoro-1-hexanol, and 1H,1H-pentadecafluoro-1-octanol. As depicted in FIG. 4a, no significant turn-on response was observed from these fluorinated analytes, which further suggests the high selectivity of U-1 MOF towards PFOA.

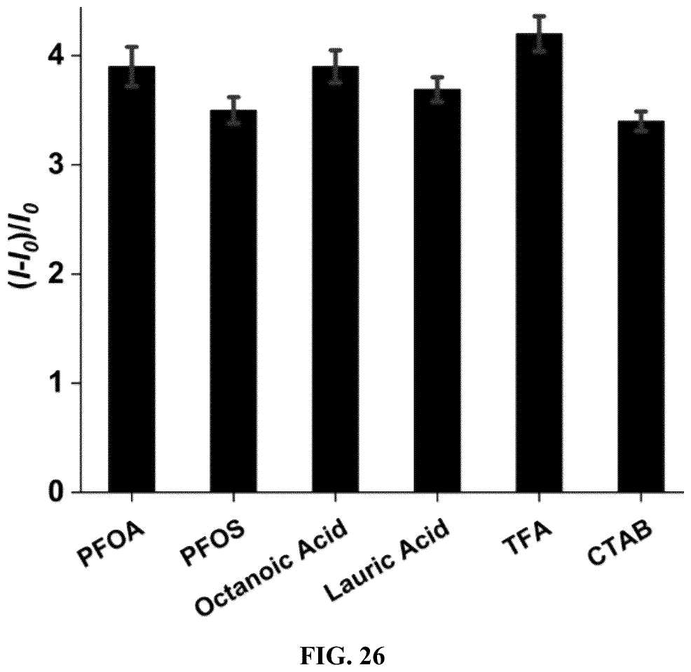

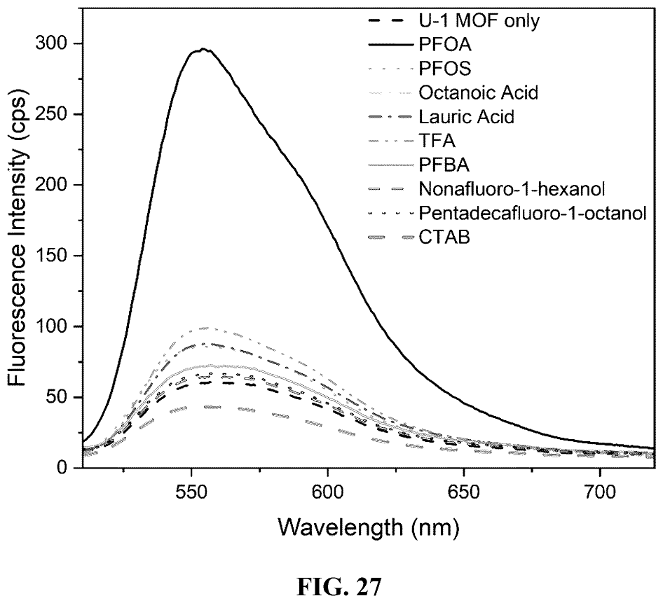

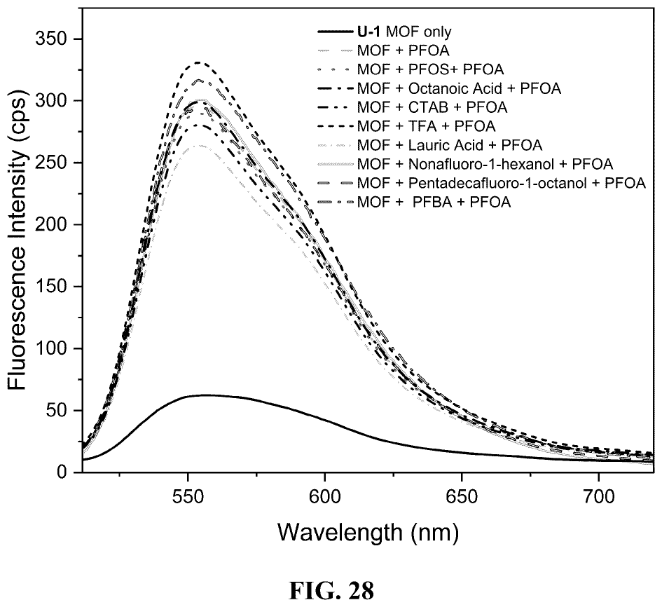

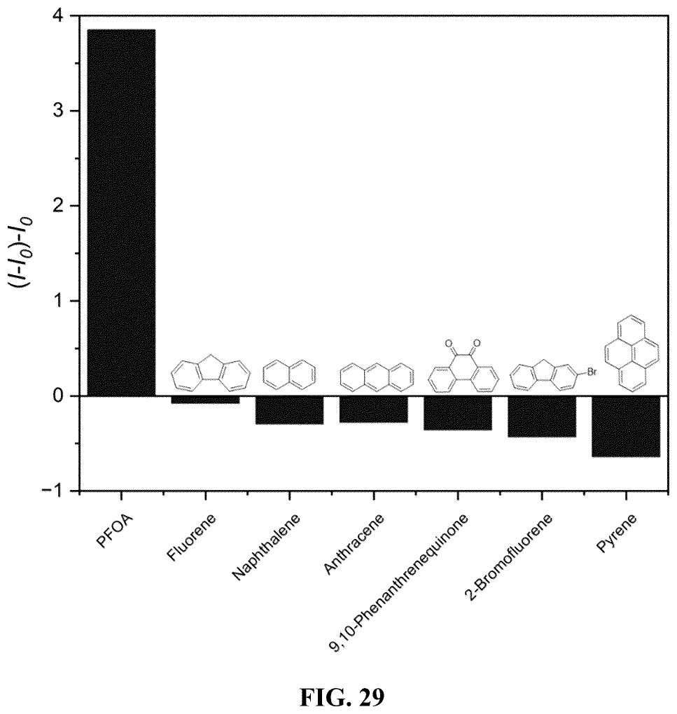

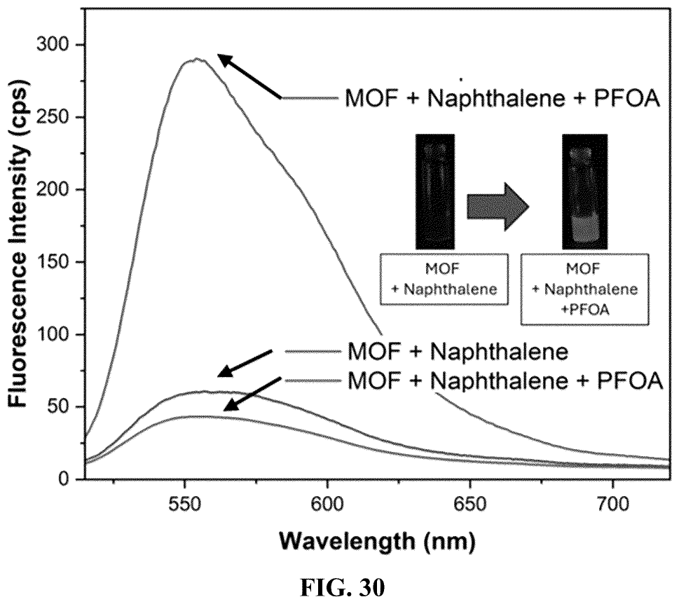

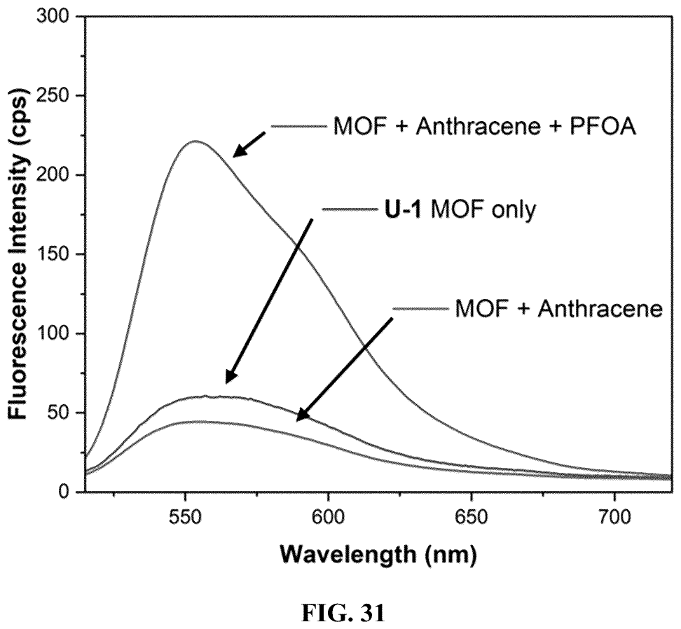

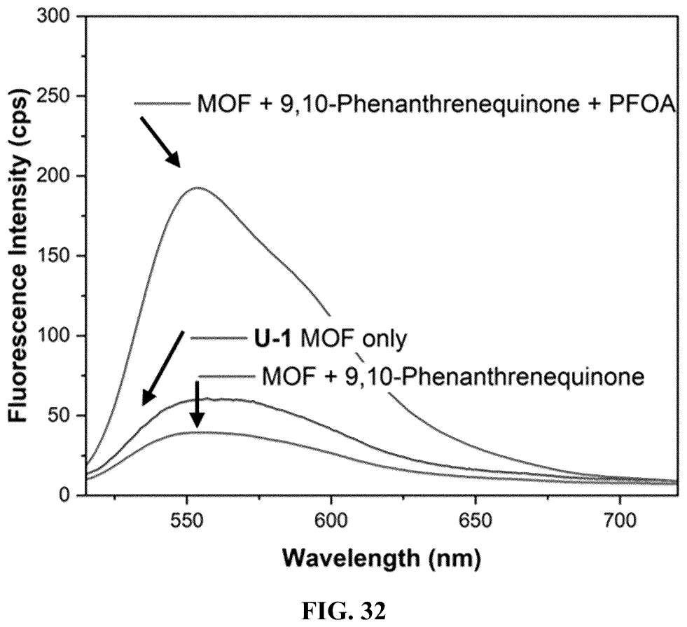

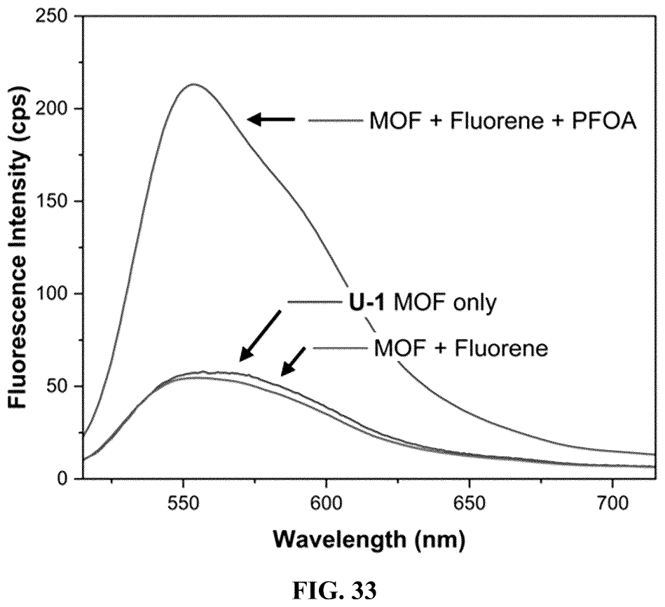

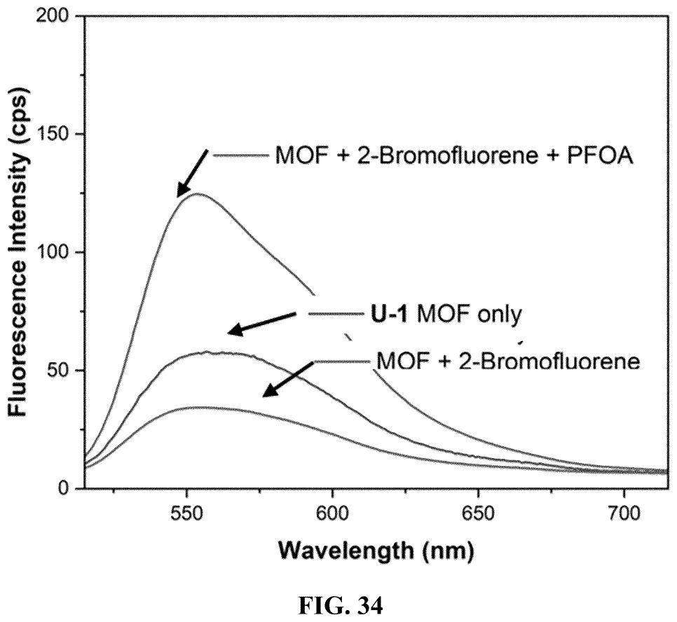

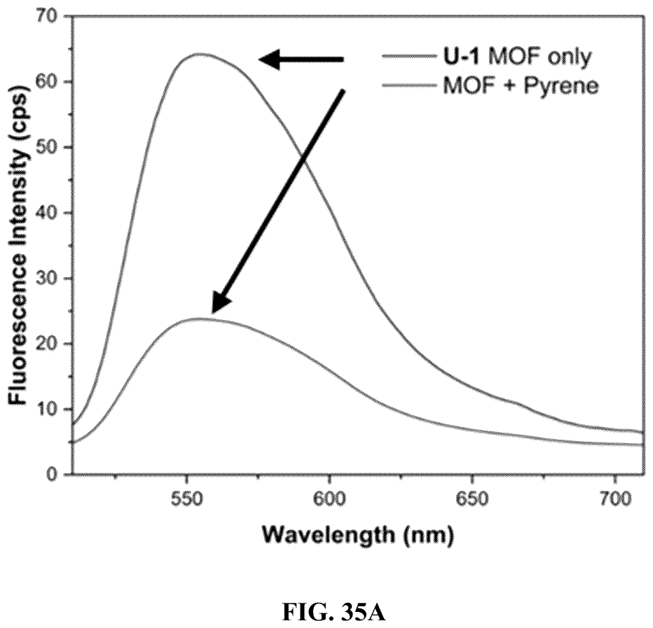

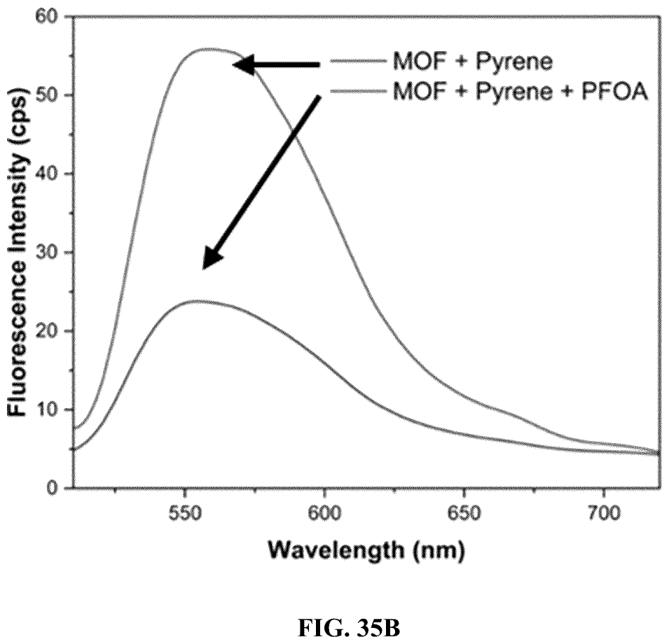

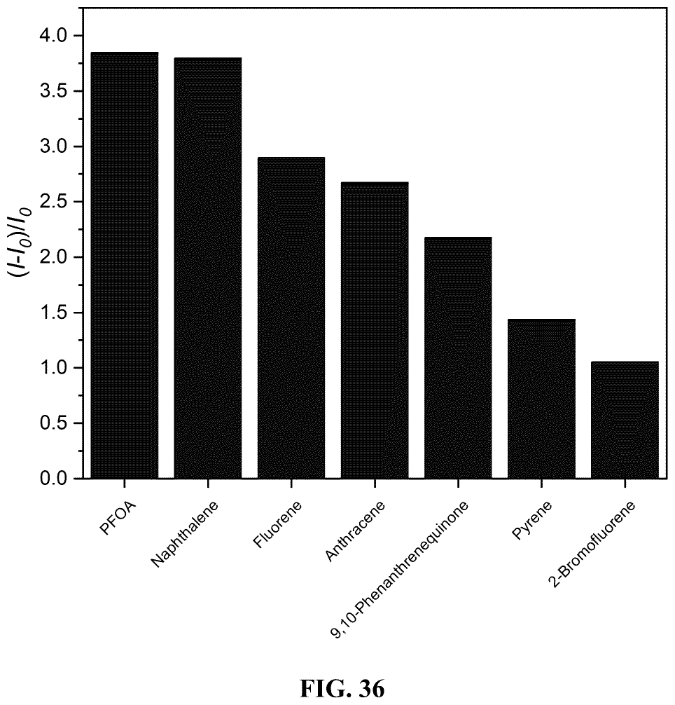

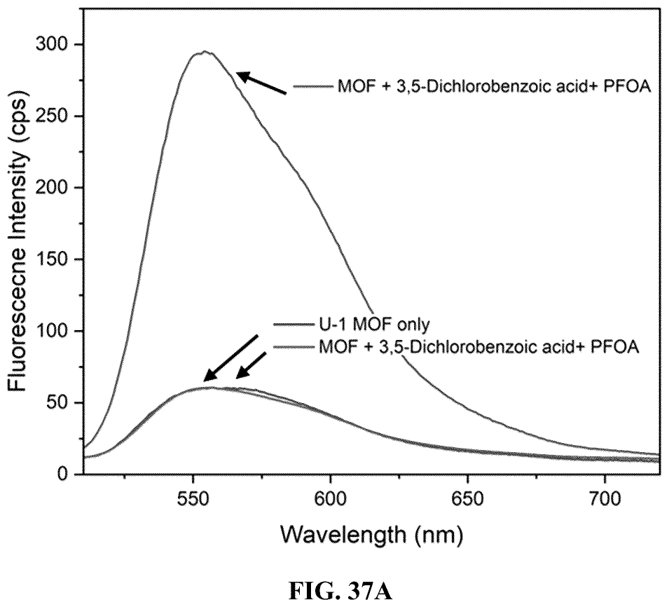

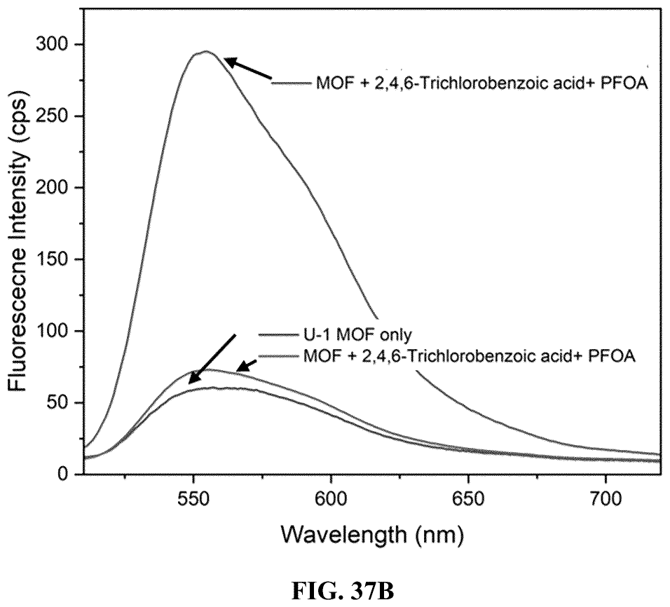

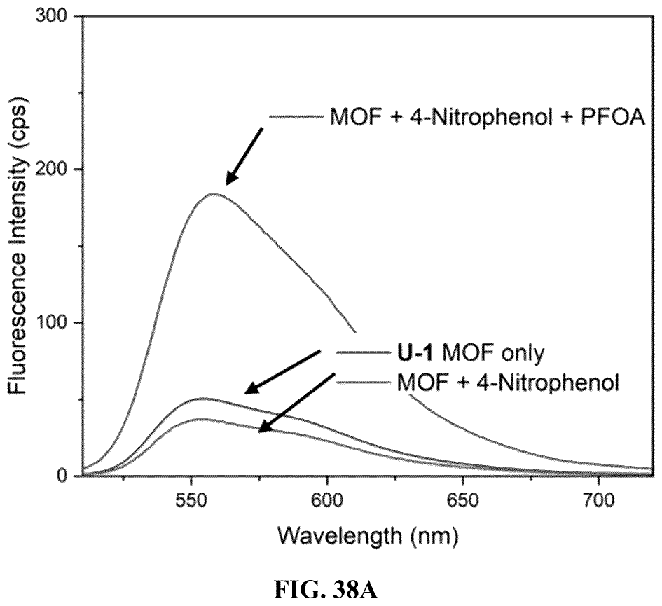

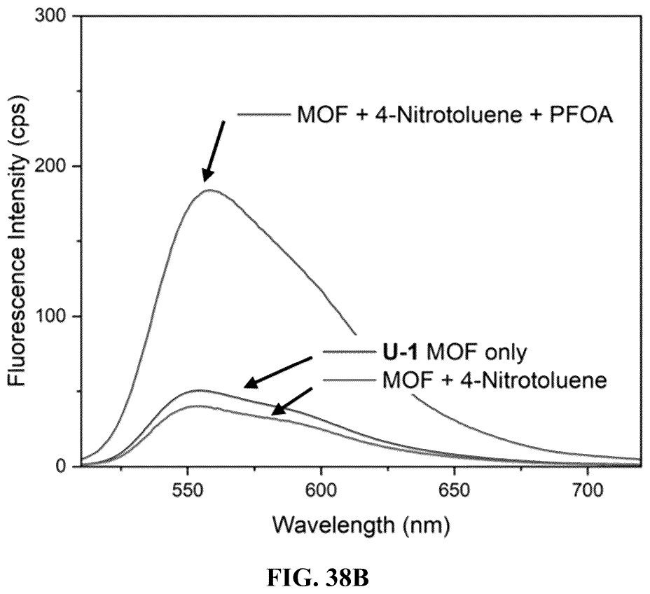

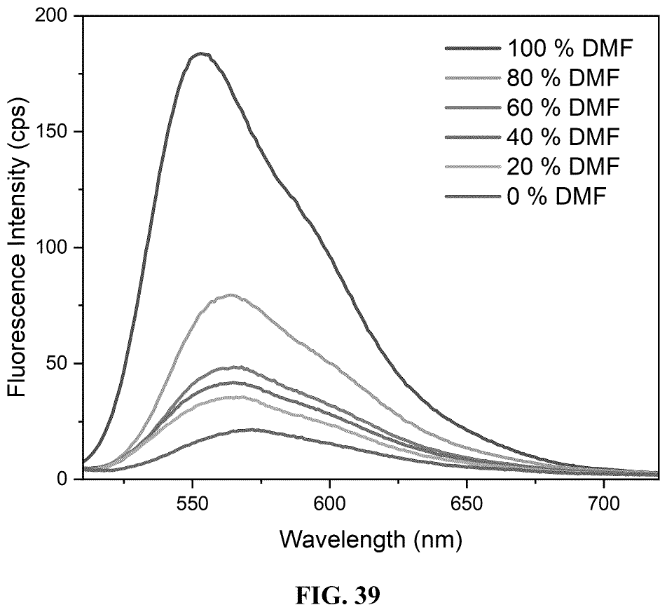

To further examine the sensitivity of U-1 MOF, another set of experiments were performed where the interferent compounds were added together with PFOA in equal amount. As depicted in FIG. 26, U-1 MOF exhibited a similar turn-on signal with no significant influence from the coexistence of interferents. The original fluorescence spectra measured for the experiments shown in FIGS. 4a and b are presented in FIG. 27 and FIG. S28. In addition to long chain acids and surfactants, polyaromatic hydrocarbons (PAHs) and their derivates, halogenated acids and nitroaromatics are also considered a major class of water pollutants. PAHs are fluorescent in nature, which can give false positive responses for any turn-on sensor. The common PAHs (naphthalene, anthracene, fluorene, pyrene) and their derivatives (e.g., 9,10-phenanthrenequinone, 2-bromofluorene) were tested along with other previously stated interferents. Interestingly, no turn-on was observed in the presence of these PAH molecules (FIG. 29). This result confirms that U-1 MOF does not show any false turn-on responses in the presence of fluorescent PAH molecules. It is also significant that even in the presence of PAH molecules, U-1 shows a significant turn-on response towards PFOA (FIG. 30-35). A significant interference from 2-bromofluorene molecule was observed (FIG. 36). The presence of a heavy atom, in this case bromine, can quench the fluorescence of the system significantly. The additional testing with halogenated acids (2,4,6-trichlorobenzoic acid and 3,5-dichlorobenzoic acid) and nitroaromatics (4-nitrophenol and 4-nitrotoluene) reveals that their presence does not significantly affect the sensing behavior of the current MOF (FIG. 37A-B and FIG. 38A-B). The chlorobenzoic acid does not alter the fluorescence of the MOF significantly, whereas nitroaromatics, well-known quenchers of MOF fluorescence, show an insignificant amount of fluorescence quenching. More importantly, MOF shows a significant turn-on response towards PFOA even in the presence of these interferences. These findings further highlight the selectivity of U-1 MOF towards PFOA, paving the way to application development in real practices, where multiple compounds coexist in water samples.

The sensing experiments were conducted in a mixture of water and DMF, while the PFOA analyte was introduced solely in water, to examine the impact of increasing the water content in the sensing system. To assess the effect of water content on the emission behavior, fluorescence spectra of the MOF were measured using different ratios of water to DMF in solution. As illustrated in FIG. S39, there was a rapid decline in the emission intensity of the MOF with increasing water content. This observation aligns with the findings of the solvent-dependent study mentioned earlier. These results strongly suggest that the turn-on response observed in the MOF is solely attributable to the presence of PFOA.

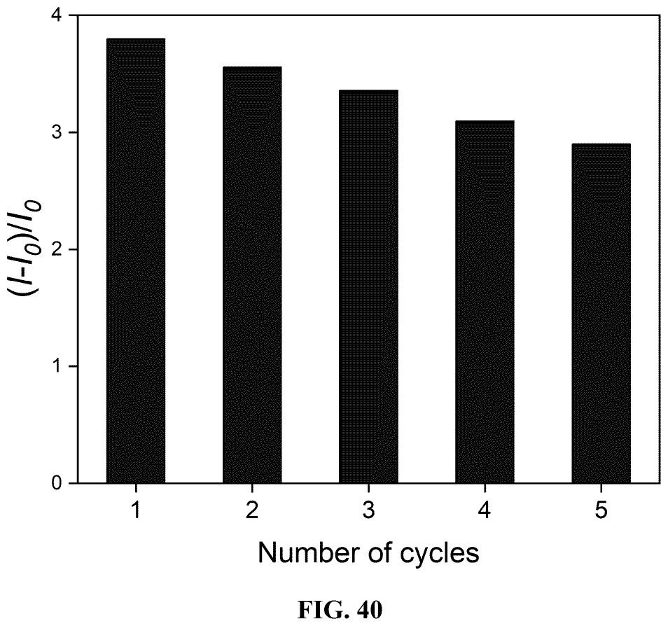

Reusability is a crucial parameter in sensor development, ensuring prolonged usage and cost-effectiveness. The reusability of the current MOF sensor was tested for up to five cycles following sensing experiments. After each sensing cycle, the MOF was recovered by washing with a 30/70 (v/v) binary mixture of 0.1 M HCl in methanol. As illustrated in FIG. 40, the MOF continues to exhibit a turn-on signal even after five sensing cycles. The slight decrease in sensitivity observed with each cycle can be attributed to the incomplete rinsing of the adsorbed PFOA, which results in relatively higher emission background (I₀).

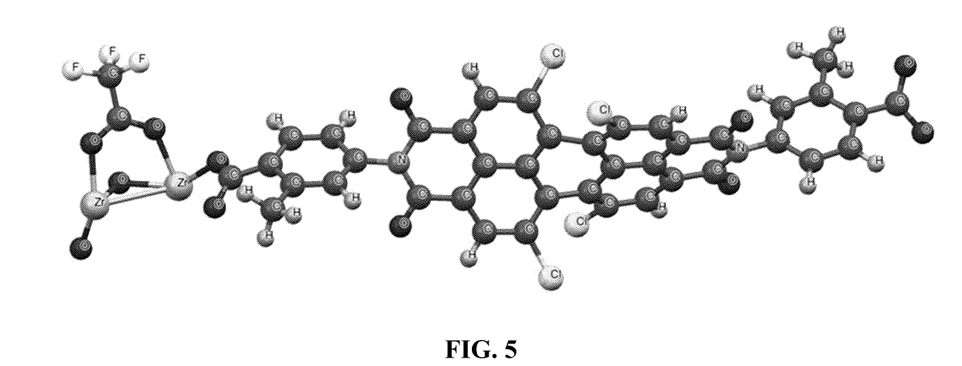

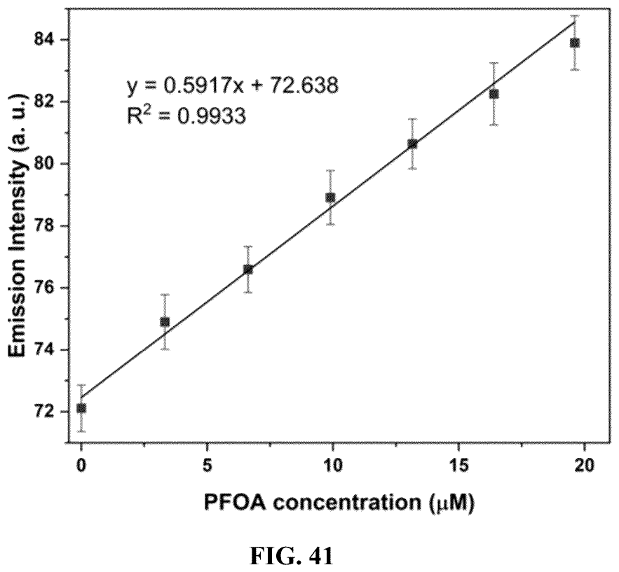

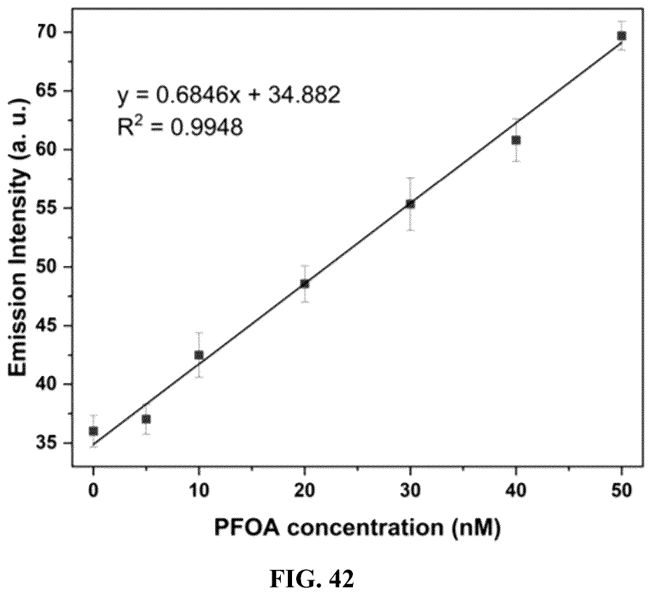

To estimate the limit of detection (LOD), the emission intensity of U-1 MOF dispersed in water/DMF (60/40 v/v) was measured at increasing concentrations of PFOA in the range of 0-M. As shown in FIG. 41, the emission intensity increases linearly with the concentration of PFOA within the testing concentration range, which yields a LOD of 1.68 μM following the IUPAC 36 criterion. To further improve the LOD, the fluorescent sensing was combined with solid phase extraction (SPE) by depositing the U-1 MOF powder within the matrix of filter paper. The cellulose fibril network of paper provides an ideal substrate for dispersing the MOF powder and forming a stable uniform film. The high porosity of MOF and cellulose substrate enables significant SPE effect that helps concentrate the PFOA compounds from low concentration samples. This allows for detection of PFOA at much lower concentrations as shown in FIG. 42. From the linear relationship between the emission intensity of MOF and the concentration of PFOA, a LOD of 3.1 nM was obtained. This low LOD is comparable with other fluorescence-based sensor materials developed recently (Table 3). For each concentration tested in FIG. 5b, a total of 75 μL aqueous solution of PFOA was slowly cast onto the MOF-coated paper so that no water extruding occurred and the evaporation of water gradually concentrated PFOA in the matrix. Depending on the evaporation temperature and time, the concentrative factor (equivalent to the volume ratio of the water before and after evaporation) can be precisely controlled, providing high measurement reproducibility. The similar SPE effect was also reported recently for a fluorescent sensor for detecting organic amines like aniline in aqueous samples, for which the LOD was also pushed down to the nM level through effective concentration by SPE. The LOD in combination with low cost and simple operation, intrinsic traits of paper-based sensors, provides great potential for the MOF materials reported herein to be further developed for practical use in quick onsite detection of PFAS. Moreover, by tuning the ligand structure and binding groups, as described herein, the reported approach can be extended to detection of other PFAS.

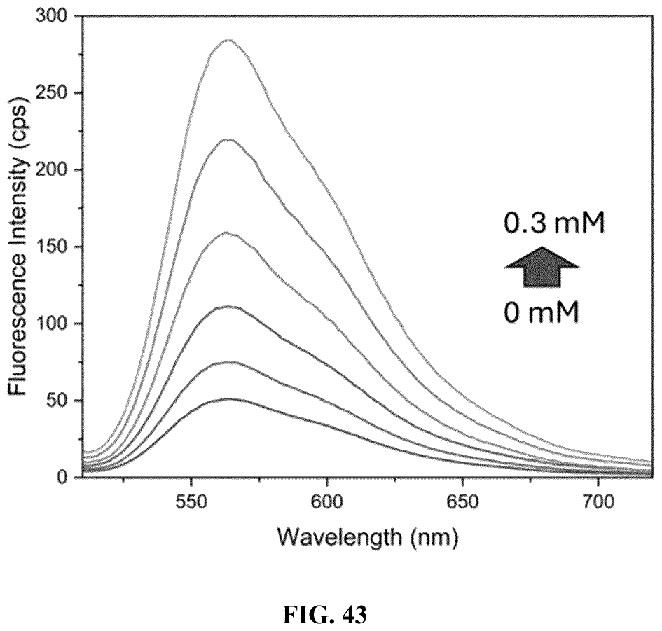

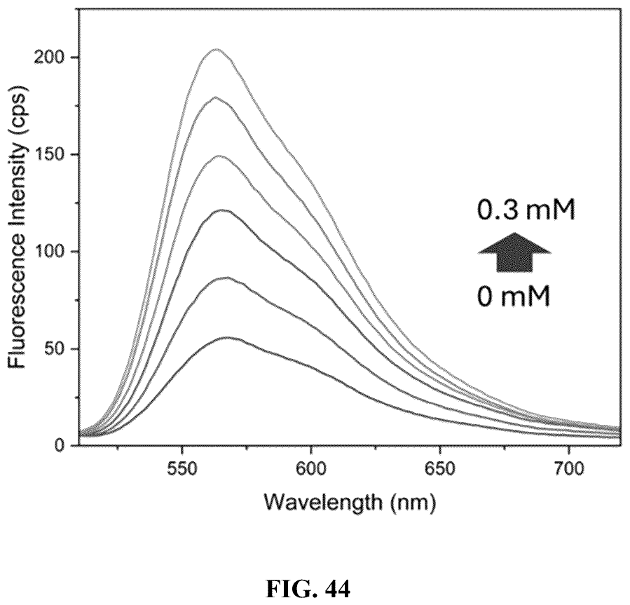

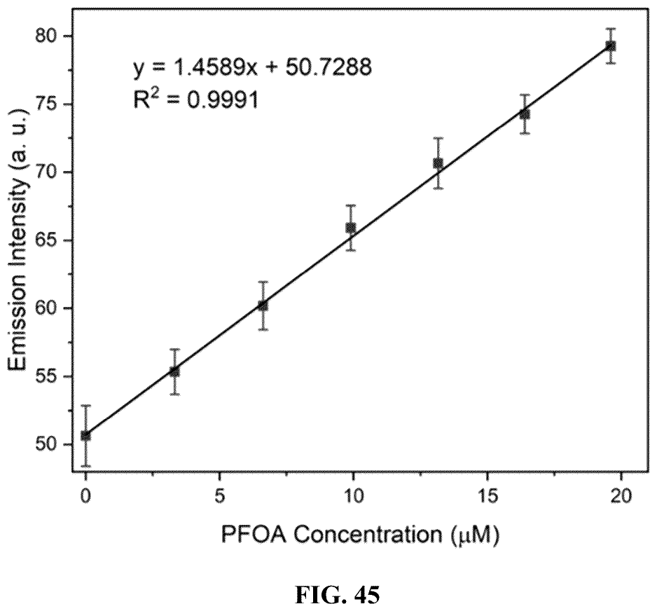

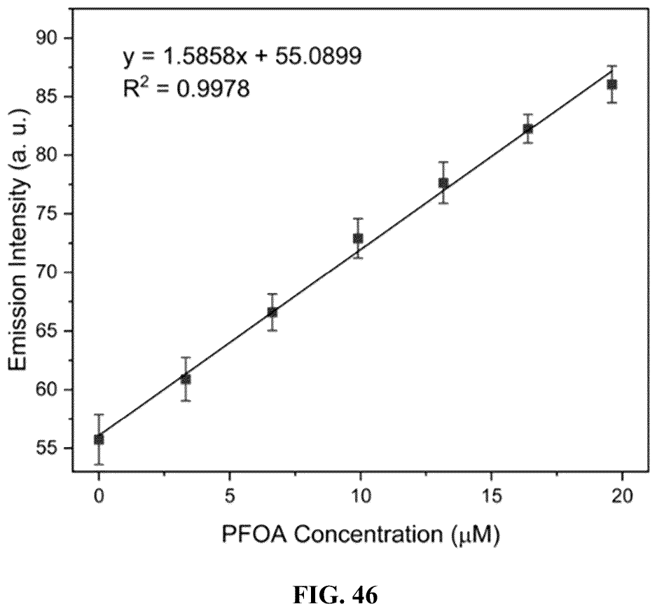

The identification of PFOA in the blood of individuals without occupational exposure to the chemical initially pointed to residential drinking water as the primary source of human exposure. Therefore, it is necessary for a PFOA sensor to be capable of operating effectively in complex, real-world systems. Sensing experiments for PFOA were conducted using tap and drinking water samples to address this need for reliability in diverse environmental matrices. The titration plot depicted in FIG. 43 and FIG. 44 indicate that the MOF can detect PFOA in both drinking and tap water. The LOD was determined to be 1.73 μM and 2.03 μM for the drinking and tap water, respectively (FIG. 45 and FIG. 46). Compared to the test with deionized water samples, the LOD obtained in drinking water remains about the same (1.68 vs. 1.73 μM), while the LOD obtained with tap water samples is slightly higher, which may be attributed to the presence of unidentified interferences (e.g., ions) in the tap water. Standard recovery experiments were performed, wherein PFOA solutions prepared in tap or drinking water were spiked as internal standards. U-1 suspensions were treated with three different concentrations of PFOA (5-, 10-, and 15 μM), and the fluorescence emission intensities were recorded. The measured concentrations closely matched the spiked concentrations of PFOA, yielding recovery percentages exceeding 91% (Table 3). These promising outcomes underscore the broad scope and practical utility of the current U-1 MOF for PFOA sensing in real-world scenarios.

TABLE 3
Detection of PFOA in real water samples.

| | PFOA | PFOA | Recovery | RSD/% |
|---|---|---|---|---|
| Water Sample | added (μM) | found (μM) | (%) | (n = 5) |
| Tap water | 5 | 4.78 | 95.6 | 2.2 |
| sample 1 | 10 | 9.34 | 93.4 | 2.1 |
| | 15 | 14.27 | 95.1 | 1.9 |
| Tap water | 5 | 4.79 | 95.8 | 2.3 |
| sample 1 | 10 | 9.39 | 93.9 | 2.4 |
| | 15 | 14.23 | 94.8 | 1.9 |
| Drinking water | 5 | 4.88 | 97.6 | 1.9 |
| sample 1 | 10 | 9.25 | 92.5 | 1.8 |
| | 15 | 14.78 | 98.5 | 2.0 |
| Drinking water | 5 | 4.57 | 91.4 | 1.7 |
| sample 2 | 10 | 9.56 | 95.6 | 1.8 |
| | 15 | 14.88 | 99.2 | 1.6 |

Mechanisms of PDOA Binding with U-1 MOF

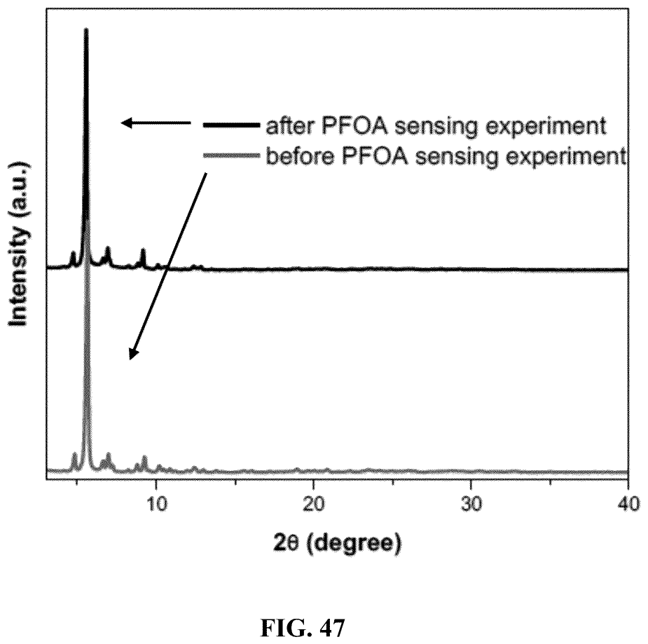

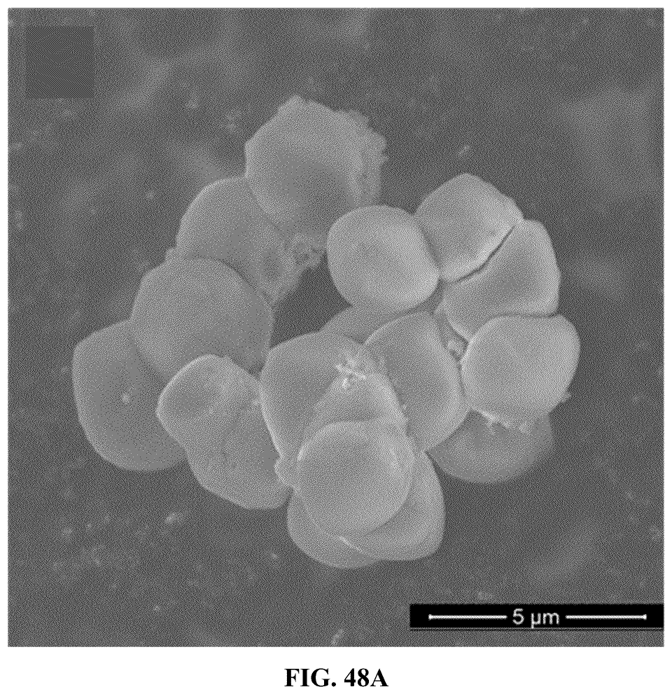

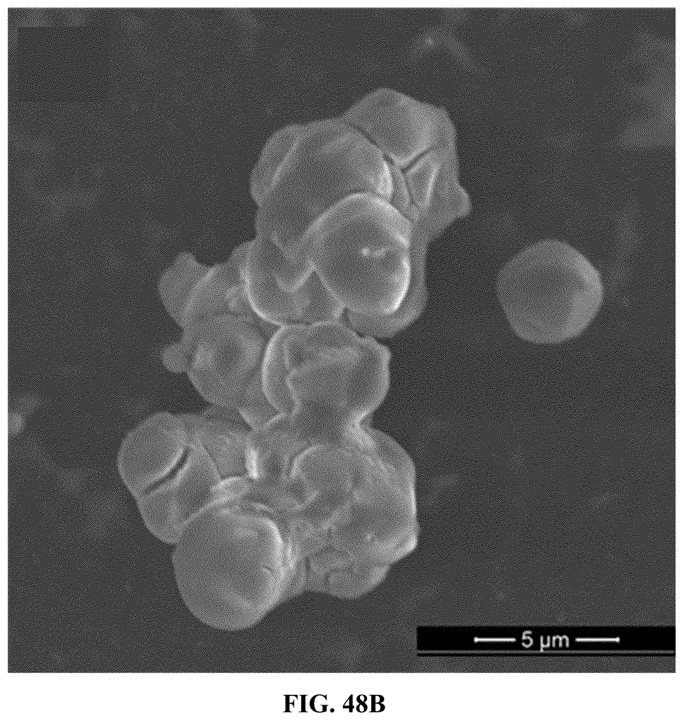

From the fluorescence sensing response obtained above for PFOA in comparison with other analytes, the binding of PFOA within MOF depends on both the complexation with the metal ion and the hydrophobic interaction with the PDI ligands. The strong binding of PFAS results in conformational change of the π-conjugation structure of PDI backbone, particularly the dihedral twisting angle around the four chloro-moieties. Such conformational change leads to an increase in emission intensity (vide infra). There is a possibility that the intercalation of PFOA may destroy the intrinsic structure of U-1 MOF, thus releasing the free ligand of PDI, leading to fluorescence turn-on. To exclude this possibility, the structural integrity of U-1 MOF powder was verified by PXRD measurement before and after the PFOA treatment. Almost no change in PXRD patterns was observed for the MOF sample after being tested with PFOA (FIG. 47), implying that the framework structure of U-1 MOF remained robust upon PFOA binding. The SEM image was acquired after the sensing experiments and compared with the MOF before PFOA sensing (FIG. 48A-B). No major changes were observed for the MOFs' shape and size, which further confirms the robustness of the MOF. Minor agglomeration of MOF particles was observed after the sensing test with PFOA. The overall hydrophobicity of the MOF may have increased by PFOA adsorption during the sensing event, leading to increased hydrophobic interactions among the MOF particles and, consequently, particle agglomeration.

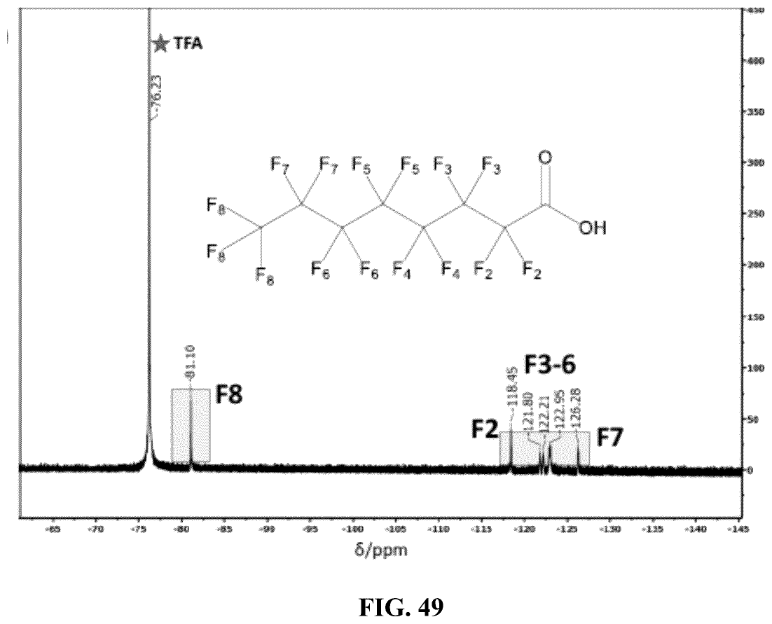

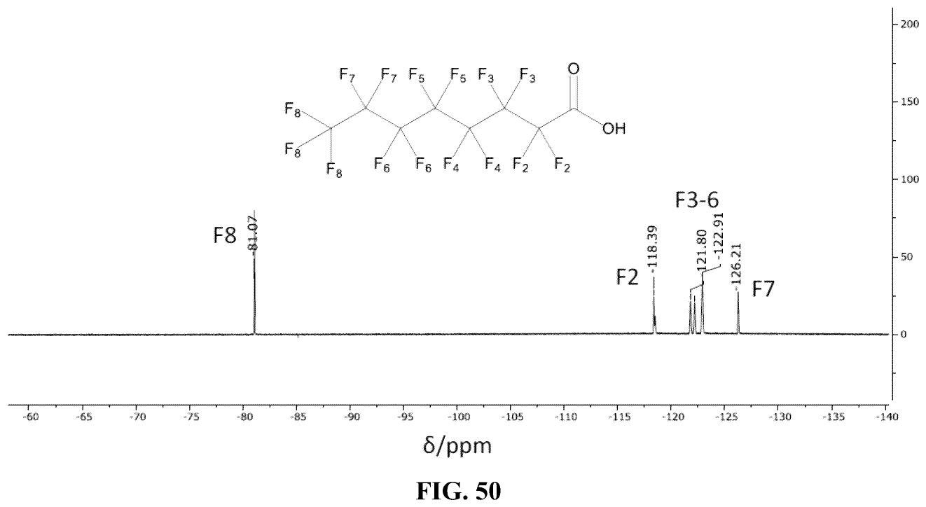

¹⁹F-NMR spectroscopy was also used to examine any inclusion of PFOA molecules inside the MOF after the sensing test. The MOF was collected after the sensing experiment. and digested with tripotassium phosphate. The ¹⁹F NMR spectrum of the digested MOF was recorded and compared with pure PFOA. As depicted in FIG. 49, the digested MOF shows a ¹⁹F NMR pattern about the same as that obtained from pure PFOA (FIG. 50), for which the peaks from −118.3 to −126.2 ppm corresponds to the fluorine atoms attached to C₂-C₇ carbons, and the peak near −81.0 ppm corresponds to chemical shifting of the terminal —CF₃ functional group. The similarity in ¹⁹F NMR pattern confirms the inclusion of PFOA into the MOF during the sensing test. Additional peak observed at −76.2 ppm in FIG. 49 suggests the presence of TFA molecules, which were used as modulator for the synthesis of U-1 MOF.

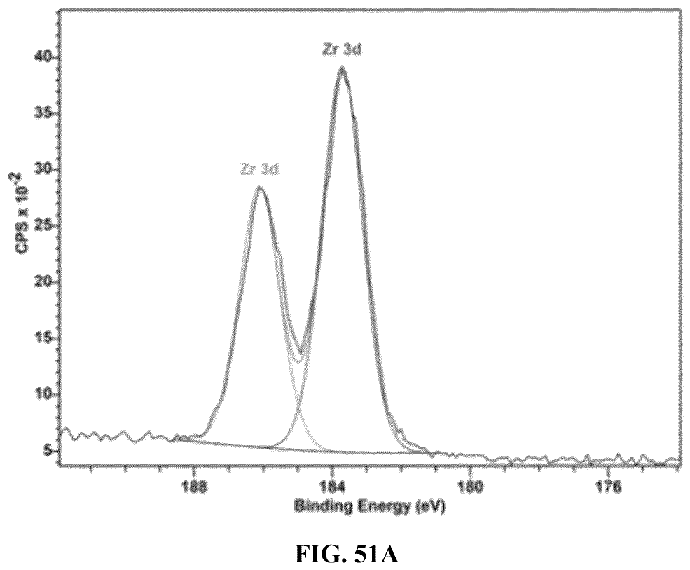

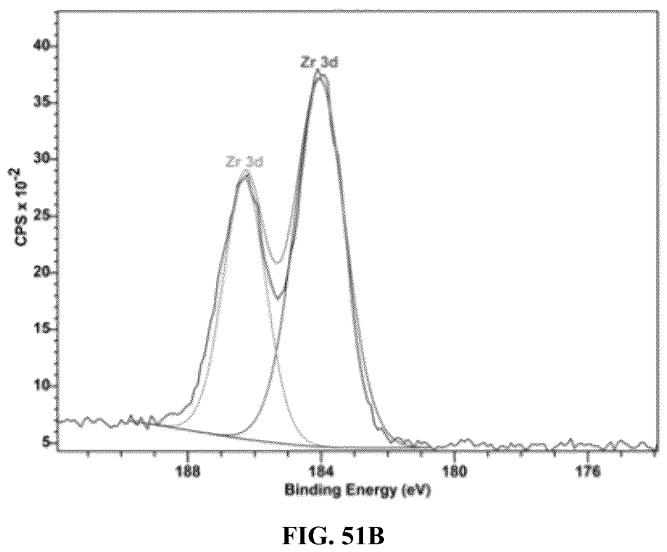

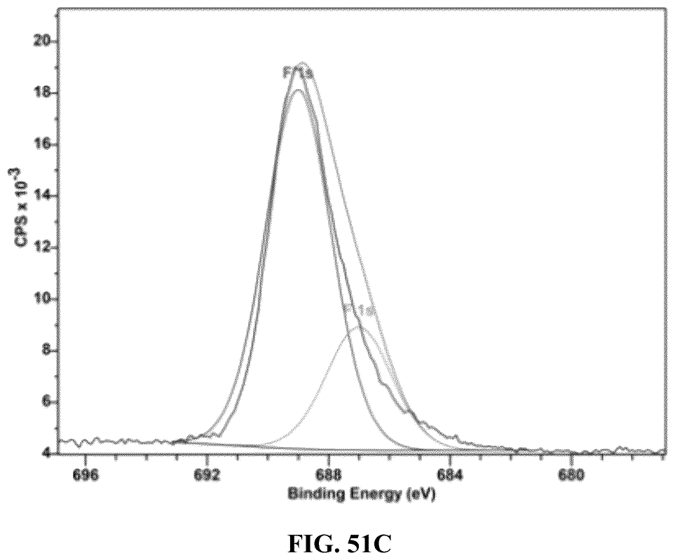

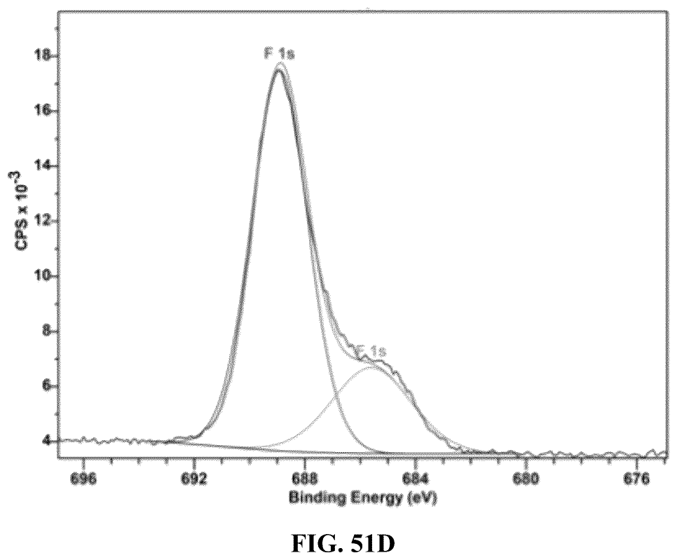

XPS was employed to analyze the surface changes of U-1 MOF upon PFOA adsorption. The XPS survey scans of U-1 MOF before and after test with PFOA, and pure PFOA are shown in FIG. 51A-D, with the data of Zr 3d and F is binding energies summarized in Table 4.

TABLE 4
Comparison table for other fluorescence-based
sensors developed in recent years for PFOA.

| | Selectivity | | | |
|---|---|---|---|---|
| | between | | Naked eye | |
| | PFOA and | LOD | visual- | Real |
| Sensor | PFOS | (nM) | ization | sample |
| Fluorinated poly(p- | No | PFOA/PFOS | No | Yes |
| phenylene ethynylene) | | 142.5/132.55 | | |
| polymer | | | | |
| Amplifying fluorescent | No | PFOA/PFOS | Yes | Yes |
| polymers (AFPs) | | 0.2/0.7 nM | | |
| Guanidinocalix [5]arene | No | PFOA/PFOS | Yes | Not |
| | | 26.4/21.4 | | tested |
| Molecular nanocage (1) | Not tested | 0.105 | No | Yes |
| Fluorescent imprint-and- | Yes | — | No | Yes |
| report sensor array | | | | |
| Aggregation-induced | No | 0.48 | Yes | Not |
| emission luminogens | | | | tested |
| MOF Sensor Array | Yes | 111.1 | Yes | Yes |
| Iridium(III) Complexed | Not tested | 20 × 10³ | No | Not |
| modified gold surface | | | | tested |
| CdS quantum dots (QDs) | Not tested | 0.3 × 10³ | No | Yes |
| Pyrene-Containing | Yes | 1.2 × 10³ | No | Not |
| Polyamines | | | | tested |
| Erythrosine B (EB)- | No | 11.8 | No | Yes |
| hexadecyltrimethyl- | | | | |
| ammonium bromide | | | | |
| (CTAB) system | | | | |
| ssDNA aptamer-based | Not tested | 0.17 × 10³ | No | Yes |
| fluorescence sensor | | | | |
| U-1 MOF | Yes | 3.1 | Yes | Yes |

The analysis reveals that U-1 MOF has characteristic Zr 3d peaks located at 183.71 and 186.11 eV, indicating the presence of Zr4+. Moreover, the F is binding energies of the MOF were found to be at 689.0 and 687 eV. Upon adsorption with PFOA, a shift in the F is binding energies was observed, with values of 688.91 and 685.60 eV, which agrees with the chemical changes induced in the trifluoromethyl groups due to adsorption in the MOF. Interestingly, the characteristic Zr 3d binding energies also shifted from 183.71 and 186.11 eV to 184.05 and 186.30 eV, respectively, indicating a perturbation in the coordination environment induced by PFOA binding (Table 5). Binding to a fluorinated compound causes the loss of electron density over zirconium, which in turn increases its 3d_{3/2} and 3d_{5/2} binding energies in the MOF. All the data presented confirm the successful adsorption of PFOA into the MOF.

TABLE 5
XPS binding energies in units of eV generated
from high-resolution scans of PFOA@U-1 and
pure samples of PFOA and U-1.

| Sample | Zr 3 d | F 1 s |
|---|---|---|
| PFOA @ U-1 | 184.05, 186.30 | 688.91, 685.60 |
| U-1 | 183.71, 186.11 | 689.00, 687.01 |
| PFOA | N/A | 688.20, 686.00 |

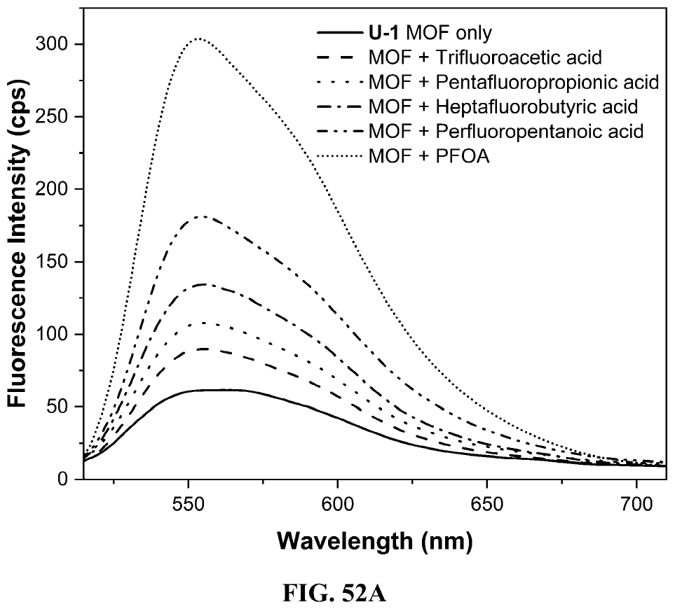

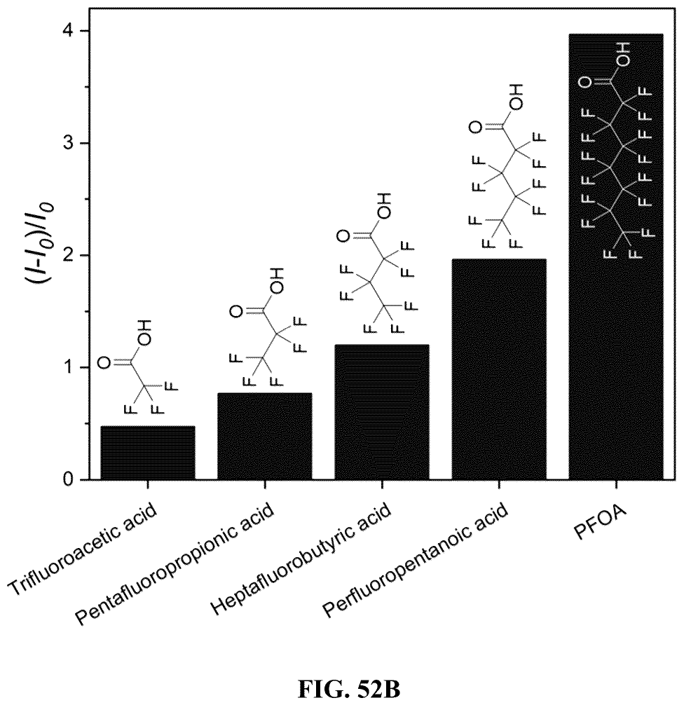

The fluorinated hydrophobic chain, along with polar carboxylic acid group, plays a pivotal role for the selective sensing of PFOA. An additional fluorescence experiment was performed to support this hypothesis. A series of short-chain perfluoroalkyl carboxylic acids (PFCAs) were treated with U-1 MOF suspension and changes in emission spectra were recorded. As depicted in FIG. 52A-B, the trifluoro acetic acid and shorted fluoroalkyl carboxylic acid show minimal responses. However, a steady increase in turn-on signal was found with increasing the length of fluoroalkyl chain. This confirms that the sensing response is dependent on both the hydrophobic chain and polar carboxylate functional group of PFCAs.

### Computation Modeling of PFOA@U-1 Interactions

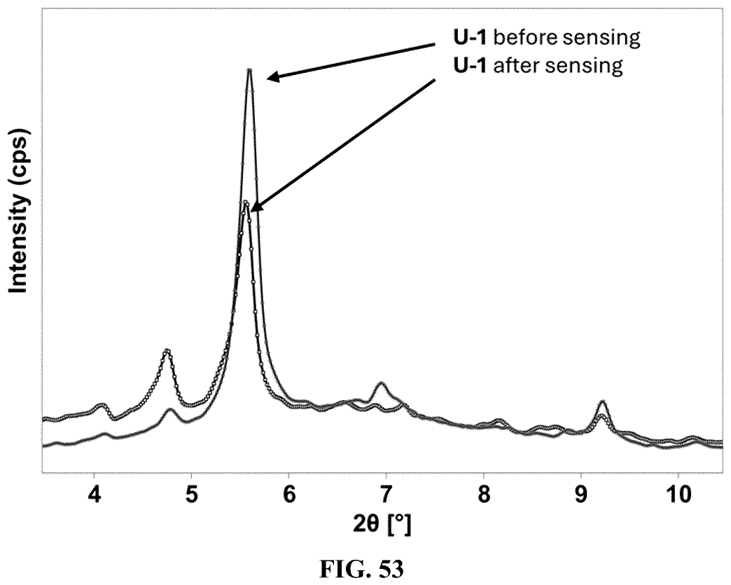

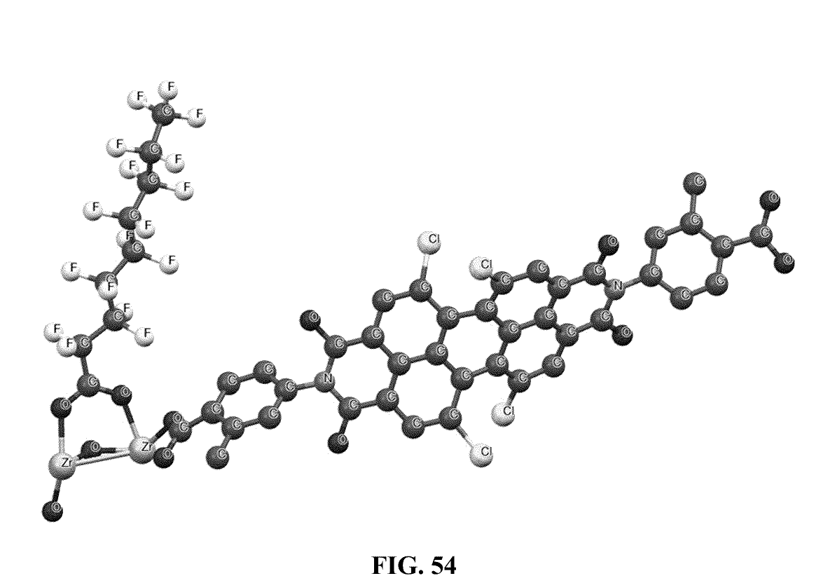

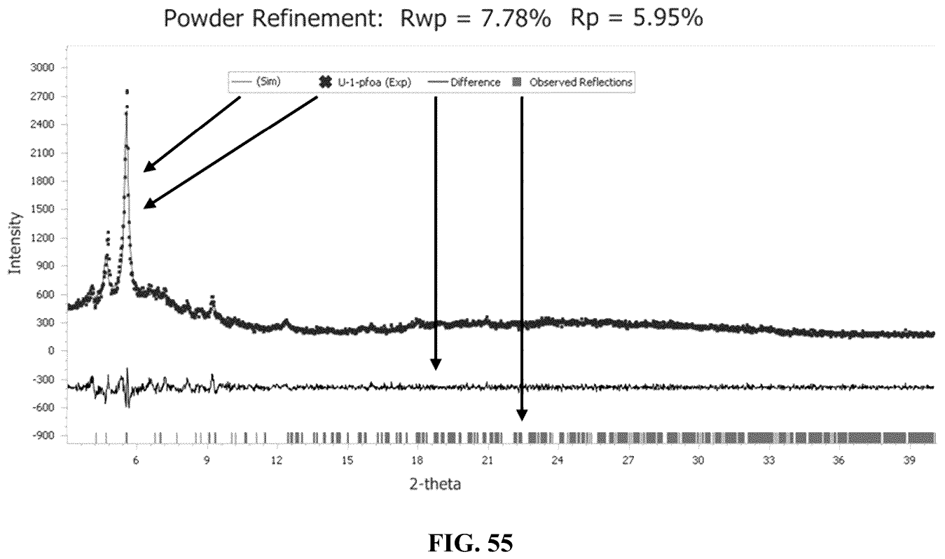

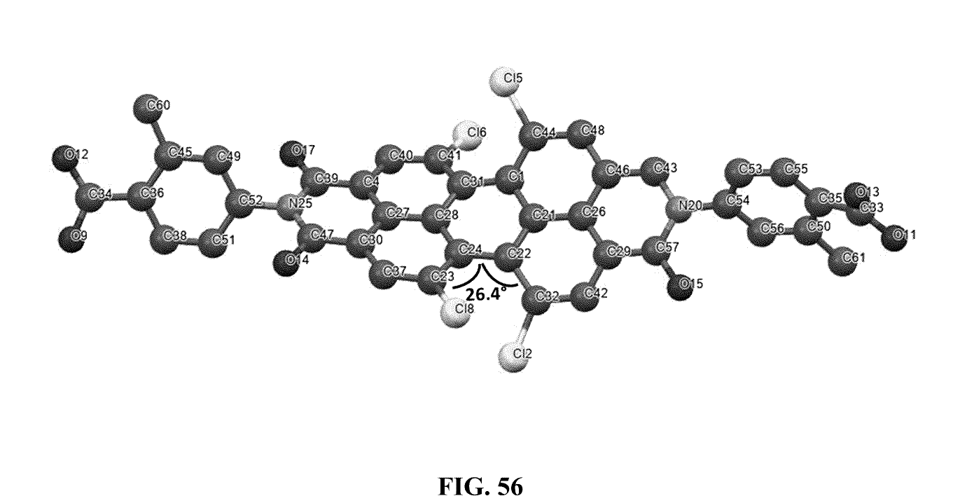

Increasing binding affinity for longer-chain perfluoroalkyl carboxylic acids (PFCAs) compared to short chain PFCAs was observed in U-1. A combined experimental and theoretical study was undertaken to understand the molecular-level interactions between MOF and PFOA, aiming to gain insights into the sensing mechanism. It is known that PFOA exhibits significantly stronger acidity than its corresponding fatty acid, implying that the interaction between PFOA and MOF could entail a covalent interaction. The shift in Bragg diffraction peak positions towards lower 20 values after PFOA sensing was confirmed by PXRD analysis (FIG. 53). This low-angle shift suggests an increase in cell volume after PFOA interaction.⁴⁸ To confirm this change, a Pawley refinement was attempted post-sensing. The new model was constructed by replacing coordinating TFA molecules with PFOA. The asymmetric unit of PFOA@U-1 contains two crystallographically independent Zr atoms, one L²⁻ ligand molecule, one PFOA molecule and two μ₃-O atoms (FIG. 54). The results of Pawley refinement revealed that the simulated and experimental PXRD data were identical for the PFOA@U-1 system (FIG. 55 and Table 6), with the cell volume expanded to 2.7%. Notably, the highly twisted PDI backbone with a dihedral twisting angle ∠(C—C—C—C) of 36.9° was drastically reduced to 26.4° after the incorporation of PFOA (FIG. 56). The PFOA-induced fluorescence turn-on can be explained by the relaxation of the twisted structure of the perylene core. The increased dihedral angle of the H₂L ligand found in U-1 MOF results in a reduced effectiveness of the overlap between the p orbital and the sp² hybridized orbital. This decreased overlap with the p orbital leads to a breakdown of conjugation within the ligand, ultimately resulting in fluorescence quenching. Conversely, after the coordinating interaction of PFOA with U-1, the MOF underwent structural changes at the molecular level and the perylene core attained more planarity in geometry. Through this, a more conjugated state is achieved. Such a change in the perylene core is likely responsible for the restoration of fluorescence and the observed turn-on response.

TABLE 6
Unit cell parameters of U-1 and U-1-PFOA

| Compound name | U-1-PFOA | U-1 |
|---|---|---|
| Space group | I 41/a | I 41/a |
| Crystal system | Tetragonal | Tetragonal |
| a = b ≠ c (Å) | a = b = 19.4763 (11), | a = b = 19.2570(6), |
| | and c = 83.1779 (16) | and c = 83.1554(7) |
| | V = 31714.2 Å³ | V = 30836.7 Å³ |

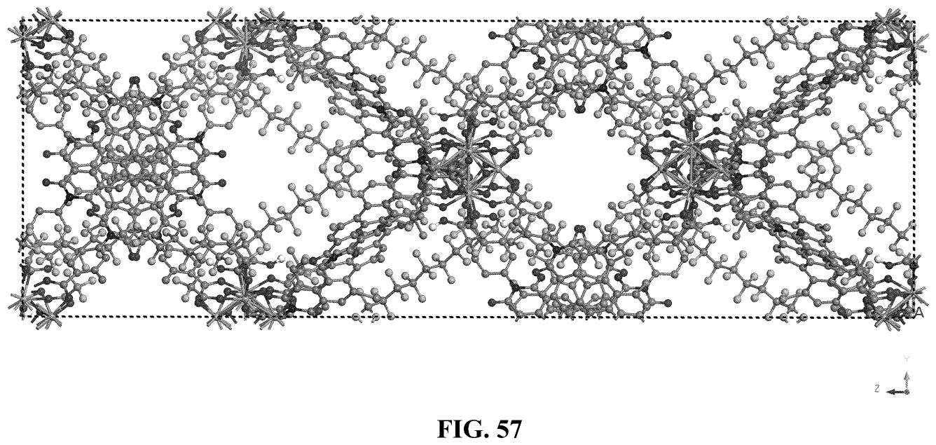

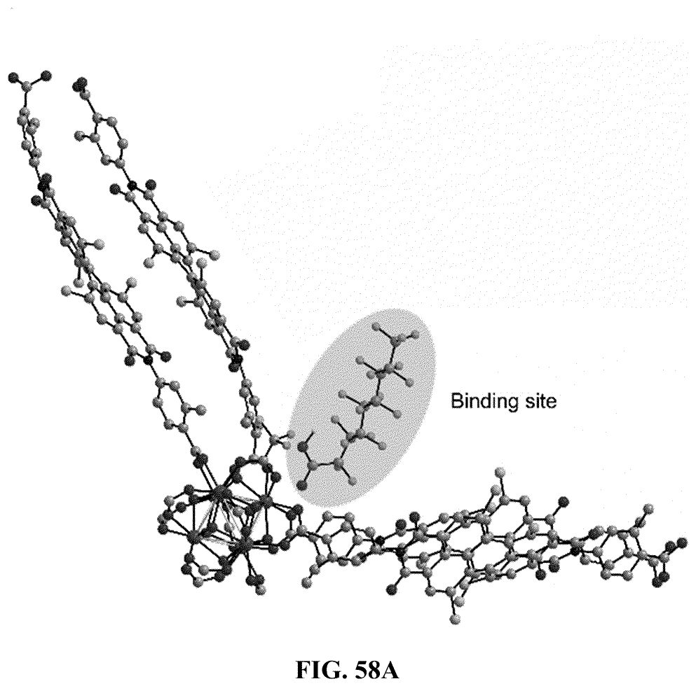

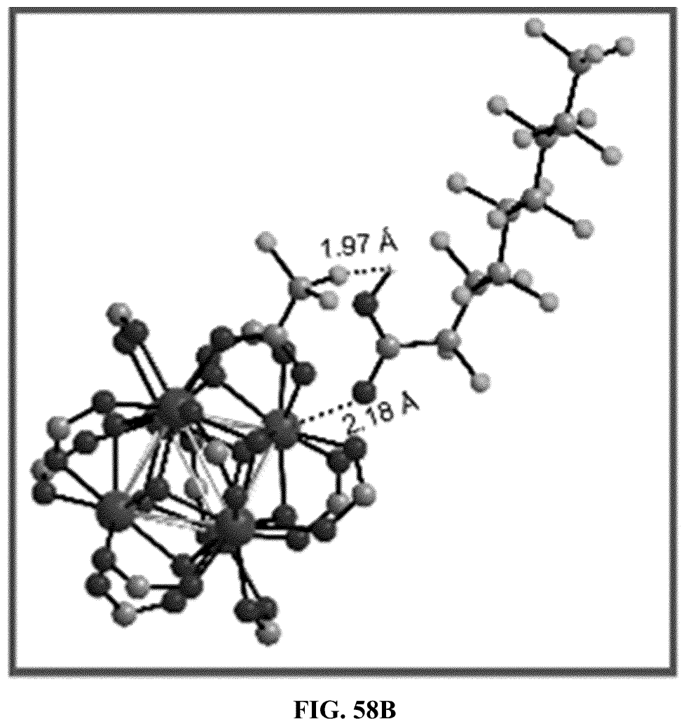

DFT calculations were further employed to investigate the origin of PFOA interactions with MOF. PFOA molecules were introduced at the adsorption sites (Zr₆ SBU) in a model of U-1 that was previously identified and confirmed by XPS and X-ray structure refinement. The resulting optimized structure of PFOA@U-1 is displayed in FIG. 57. The Zr₆ node at the adsorption site features a Zr-coordinated TFA molecule that enables hydrogen bonding interactions with PFOA via —CF₃ functional groups, while Zr sites promote additional coordinative bonding. The adsorption of PFOA near the Zr₆ node is visualized in FIG. 58A-B, showing the PFOA@U-1 system is stabilized by strong H-bonding interactions (RCOOH . . . F—C, 1.97 Å), accompanied by strong coordinate bonding (RCOOH—Zr, 2.18 Å). Such strong coordinate bonding is in line with the proposed complex formation mechanism as derived from experimental data.

### CONCLUSION

Described herein is the synthesis and systematic exploration of robust, fluorescent PDI-based MOF, namely U-1, for the detection of PFOA through a fluorescence turn-on response. The tetrachloro-substitution increases the solubility of the PDI ligand during MOF synthesis, whereas the judicious incorporation of methyl groups increases the hydrophobicity around the Zr₆ SBUs in the MOF. This minimizes the interaction with water molecules, which in turn helps enhance the stability of the MOF in water. Zirconium metal ion were selected for the MOF synthesis because of its binding affinity towards PFAS molecules, like PFOA, that contain carboxylic groups. The PDI-based MOF showed a rapid fluorescence turn-on response towards PFOA in aqueous solutions. Although similar in structure, PFOS did not demonstrate a significant turn-on response compared to PFOA. This reusable MOF sensor has the ability detect the presence of PFOA in real water samples, including tap and drinking water. The rapid turn-on sensing via a MOF-PFOA complex formation mechanism may soon lead to this material being an efficient dual sensor and absorber for PFAS.

The results described herein reveal that U-1 MOF exhibits outstanding stability in various chemical environments. The fluorescent sensing of U-1 MOF is relatively selective toward PFOA as tested in solution phase. The same fluorescence turn-on response was also observed for the solid phase by depositing the MOF onto a filter paper. Built upon the effective SPE effect, the LOD of the film sensor was pushed down to 3.1 nM. Detailed structural analysis suggests that the fluorescence turn-on is predominated by two cooperative interactions, the complexation between the carboxylic group of PFOA and the zirconium metal center, and the hydrophobic interaction between the fluorinated alkyl chain of PFOA and the PDI ligand. The work presented herein showcases the great potential of PDI-based MOFs as fluorescent probes for the sensitive and selective detection of PFAS, for which the detection selectivity can be further improved by tuning the ligand structure and the porosity of MOFs.

### Example 2: UiO-66-Based Cationic MOFs for Detection of PFAS

High adsorption capacity for perfluoroalkyl or polyfluoroalkyl substances (PFAS) is crucial for the dual functionality of MOF of detecting and removing PFAS. Described herein is the design and synthesis of a non-fluorinated MOFs contain UiO-66-based bridging ligands that display adsorption with enhanced adsorption capacity as compared to commercial granular activated carbon (GAC), a common type of sorbent for PFAS and other water pollutants.

### Exemplary Synthesis

UiO-66-N(CH₃)₃⁺. UiO-66-NH₂ was performed by the literature procedure, as reported by Dalapati, R. and Biswas, S. (Dalapati, R. and Biswas, S., Sensors and Actuators B: Chemical, 2017, 239, 759-767). UiO-66-NH₂ (0.5 g) was added in a mixture of CH₃I (3 mL) and toluene (15 mL). The mixture was transferred into Teflon lined autoclave and heated at 80° C. for overnight for the partial quaternization process. The resulting dark yellow product, denoted as UiO-66—N(CH₃)₃⁺, was subsequently recovered through filtration, subjected to methanol washing, and ultimately dried at 60° C. This process substantially diminishes the reaction duration in comparison to earlier reported methodologies, which typically required a period of three days.

MOF UiO-66-N(CH₃)₃⁺. UiO-66-NH₂ MOF was further modified via a previously reported procedure with modifications (Xiaocong, T. et al., Chem. Commun., 2023, 59, 4507-4510). UiO-66-NH₂ (0.5 g) was added in a mixture of CH₃I (3 mL) and toluene (15 mL). The mixture was transferred into Teflon lined auto-clave and heated at 80° C. for overnight for the partial quaternization process. The resulting dark yellow product, denoted as UiO-66-N(CH₃)₃⁺, was subsequently recovered through filtration, subjected to methanol washing, and ultimately dried at 60° C.

A completely dried sample (50 mg) of UiO-66-N(CH₃)₃⁺ was soaked in the 5 mM aqueous solution (50 mL) of sulforhodamine B (SRB) dye for 1 h. The resulting solid was washed vigorously with water until no fluorescence was observed in supernatant solution and isolated via centrifugation.

### Quantitative Analysis of PFOA Adsorption

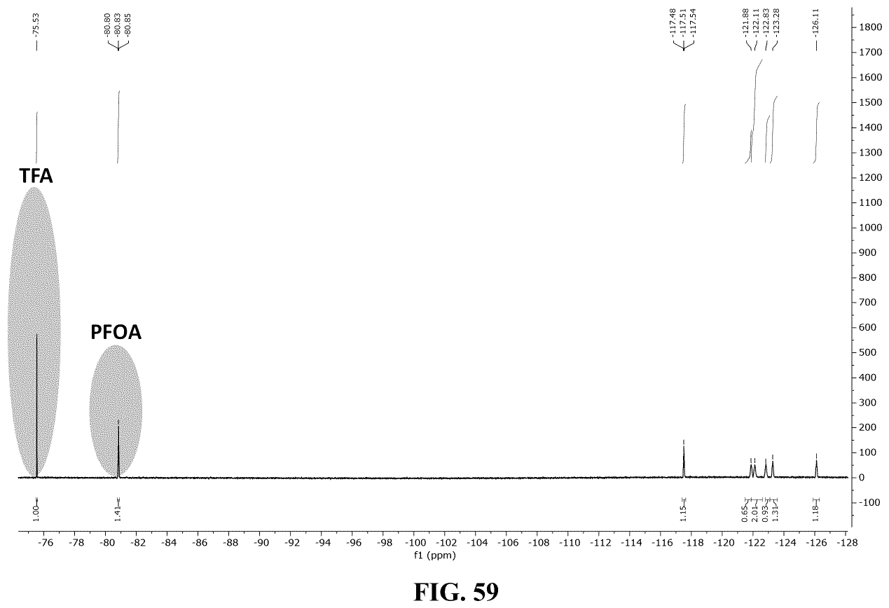

Adsorption studies were performed by adding ˜10 mg of UiO-66-N(CH₃)₃⁺ to 20 mL of 1000 ppm PFOA solution in a polypropylene centrifuge tube at room temperature. The solution was centrifuged after 30 min of stirring and 700 μL of solution was transferred to NMR tube. A 50 μL TFA/D₂O was added as internal standard to the NMR tube before analysis. Quantitative ¹⁹F NMR (FIG. 59) utilizing a 15 s relaxation delay to ensure reliable integration coupled with eq 1 was used to determine the concentration of PFOA before and after adsorption.

$$[ PFOA ] = \frac{{I}_{PFOA}}{{I}_{TFA}} \frac{{N}_{TFA}}{{N}_{PFOA}} [ TFA ] \qquad ( 1 )$$

Here, [PFOA] and [TFA] are the respective concentrations, I_{PFOA} and I_{TFA} are the integrals from the ¹⁹F-NMR spectrum, respectively, and N_{TFA} and N_{PFOA} are the number of F atoms that give rise to each NMR signal, respectively. The resonance at −75.33 ppm is due to the —CF₃ group of TFA, while the resonance at −80.82 ppm arises from the —CF₃ group of PFOA. The difference in PFOA concentration before and after adsorption is assumed to be adsorbed by UiO-66-N(CH₃)₃⁺.

### MOF Structural Analysis

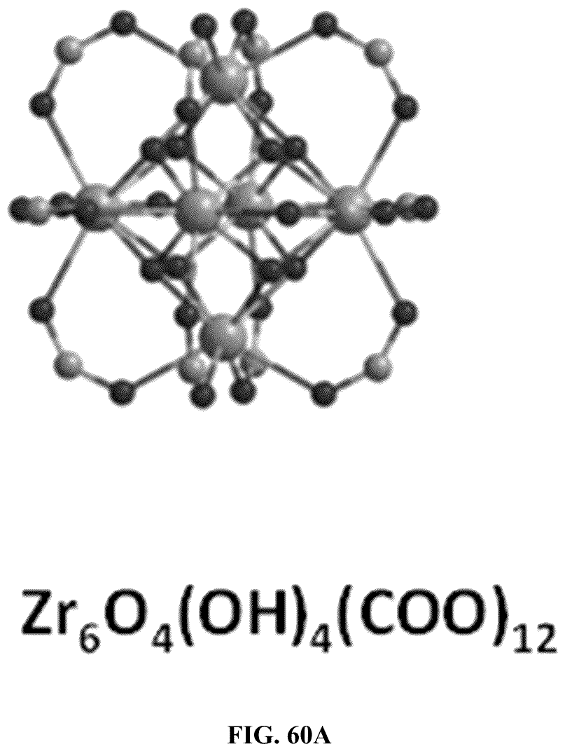

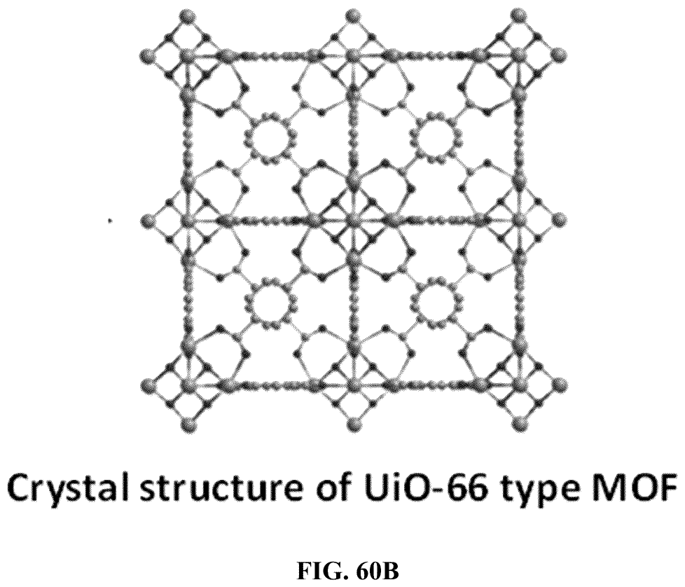

Current post-synthetically modified UiO-66-N(CH₃)₃⁺ MOF has similar structure like parent UIO-66-NH2. The structure consists of an inner Zr6O4(OH)4 core in which the triangular faces of the Zr₆-octahedron are alternatively capped by μ₃-O and μ₃-OH groups. All the edges of the polyhedron are connected by carboxylate groups (μ2-(CO2)) from dicarboxylic acids, forming a Zr6O4(OH)4(CO2)12 cluster. Each zirconium atom is thus eight-coordinated, adopting a square-antiprismatic coordination geometry. The ligands are bonded exclusively through oxygen atoms. One square face of the antiprism is constituted by oxygen atoms from the carboxylate groups, while the other square face comprises oxygen atoms from the μ3-O and μ3-OH groups. The structure is shown by FIG. 60A-B.

### Characterization of UiO-66-NH2 MOF

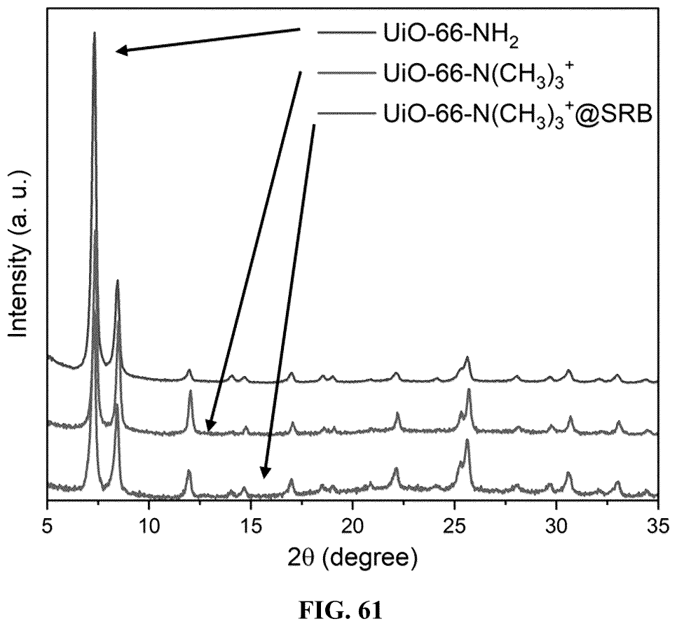

The synthesis of UiO-66-NH₂ MOF was first confirmed by powder XRD analysis. Further modification of MOFs was also confirmed by XRD analysis. As shown in FIG. 61, MOF can retain its crystallinity after modification, which confirms that MOF is stable enough for further experiments.

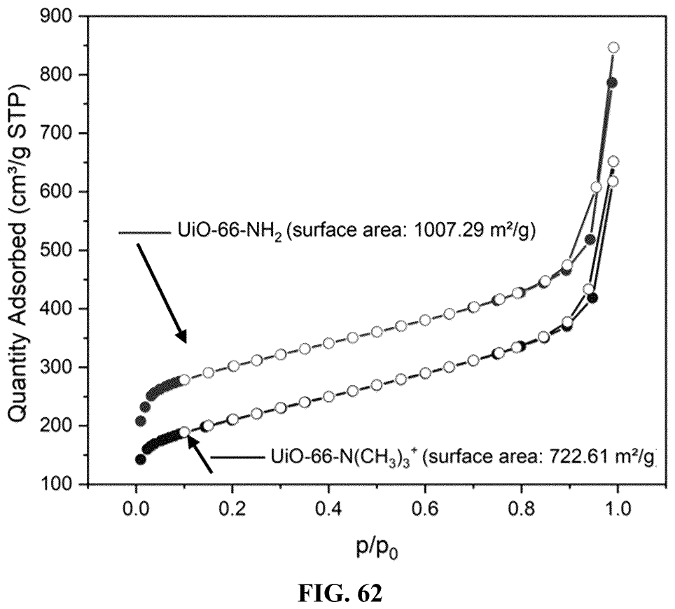

The porosity of post synthetically modified cationic MOF was also measured by BET surface area analysis. Data shown in FIG. 62, shows that cationic UiO-66-N(CH₃)₃⁺ MOF shows a less surface area of 722 m²/g compared to unmodified UiO-66-NH₂ MOF. Such reduction of surface area can be explained by the introduction of bulky methyl functional group in the MOF by post-synthetic modification.

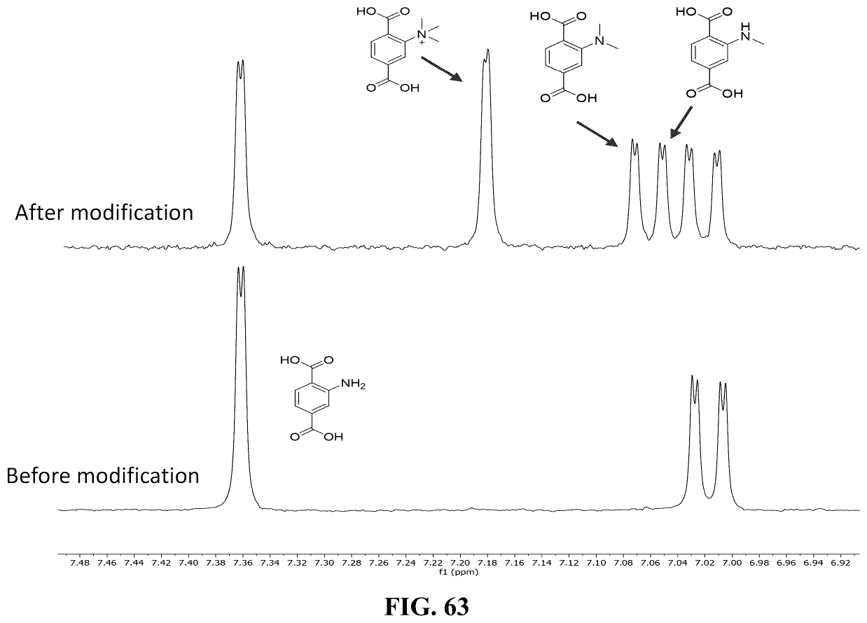

Characterization by ¹H NMR found that UiO-66-N(CH₃)₃⁺ contains three types of methylated ligands, including secondary, tertiary, and quaternary amines. As the ¹H NMR spectrum of UiO-66-NH₂ depicted in FIG. 63, proton peaks at 7.00, 7.02 and 7.36 ppm were attributed to 2-aminoterephthalate ligand. The newly appearing proton peaks (δ=7.05 for secondary amine, δ=7.07 for tertiary amine and δ=7.18 for quaternary amine of UiO-66-N(CH₃)₃⁺ suggests the successful functionalization of UiO-66-amine.

### Adsorption Testing Results of PFOA

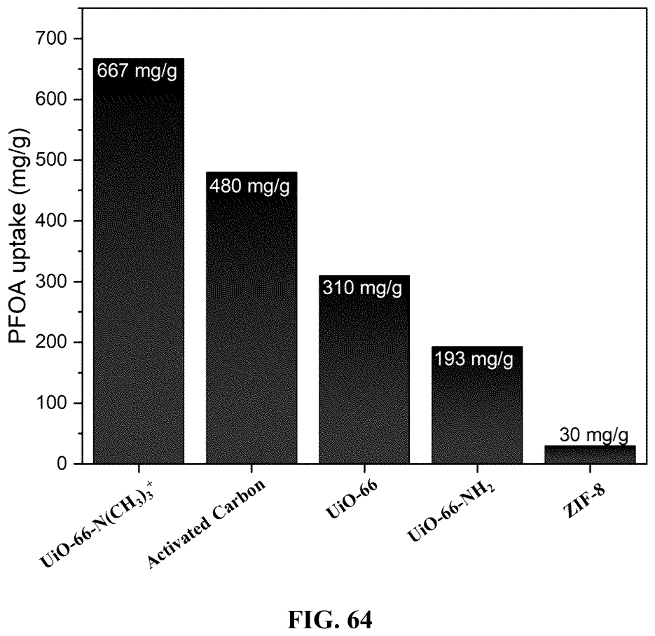

The adsorption capacity of PFOA was tested by cation modified MOF for the first time. The adsorption capacity was quantified by ¹⁹F-NMR spectra analysis as described earlier. In addition to cationic MOF, other well-known MOFs and commercially available activated carbon were also used to compare the PFOA uptake capacity (FIG. 64). It was observed that, catatonically modified UiO-66-N(CH₃)₃⁺ MOF can uptake highest PFOA (667 mg/g), which is almost 3.4 times higher than UiO-66-NH₂ (193 mg/g). It is worth mentioning that UiO-66-NH₂ MOF has a higher surface for interaction with PFOA area compared to UiO-66-N(CH₃)₃⁺ MOF. Despite this fact, cationic modified MOF interacts with a greater number of PFOA molecules than unmodified and non-cationic MOFs. This confirms that cationic modification of MOF has a great impact on PFOA uptake. The UiO-66-N(CH₃)₃⁺ MOF shows a ˜1.4 times and 2.1 times higher PFOA uptake compared to activated carbon material and well known UiO-66 MOF, respectively. Another known MOF, ZIF-8 does not show any promising PFOA uptake.

What is claimed:

1. A composition comprising at least one metal-organic framework (MOF), or salt thereof, the metal-organic framework comprising:
a plurality of metal-containing secondary building units (SBU) linked together by one or more bridging ligands,
wherein said bridging ligands, or salts thereof, have a formula (I): 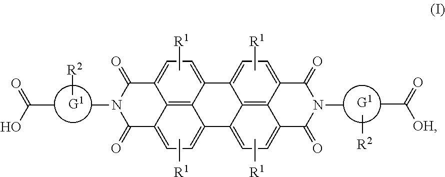
wherein
R¹, at each occurrence, is independently hydrogen, C₁₋₈alkyl, halo, —O(R^{1a}), —N(R^{x})ₘ, or —S(O)₂OH;
R², at each occurrence, is independently hydrogen, C₁₋₈alkyl, —O(R^{1a}), or C₁₋₈alkyl-OH and wherein every R² is not hydrogen;
G¹, at each occurrence, is independently phenyl, a 6- to 12-membered aryl, or a 5- to 12-membered heteroaryl, wherein the 5- to 12-membered heteroaryl is optionally substituted at a heteroatom with hydrogen or C₁₋₈alkyl;
R^{1a}, at each occurrence, is independently hydrogen or C₁₋₈alkyl, or phenyl;
R^{x}, at each occurrence, is independently hydrogen or C₁₋₃alkyl; and
m=2-3.

2. The composition of claim 1, wherein G¹, at each occurrence, is phenyl.

3. The composition of claim 1, wherein the bridging ligands, or salts thereof, have a formula (Ia):

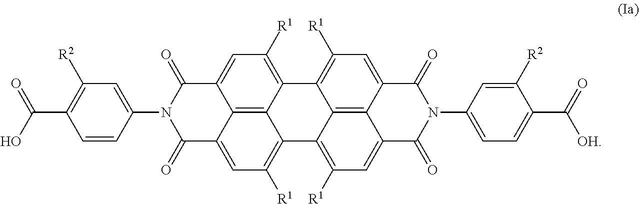

4. The composition of claim 1, wherein R¹, at each occurrence, is halo.

5. The composition of claim 1, wherein R¹, at each occurrence, is chloro.

6. The composition of claim 1, wherein R², at each occurrence, is C₁₋₈alkyl.

7. The composition of claim 1, wherein R², at each occurrence, is C₁alkyl.

8. The composition of claim 1, wherein the bridging ligands, or salts thereof, are selected from:

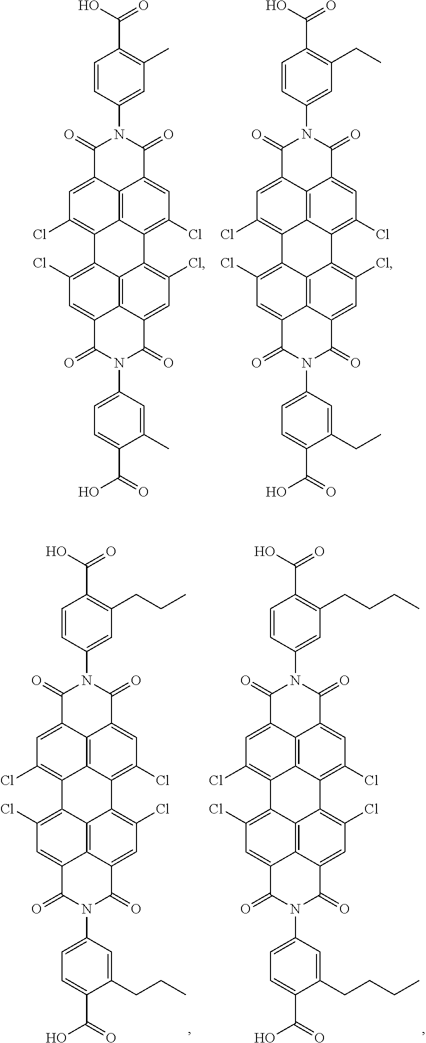

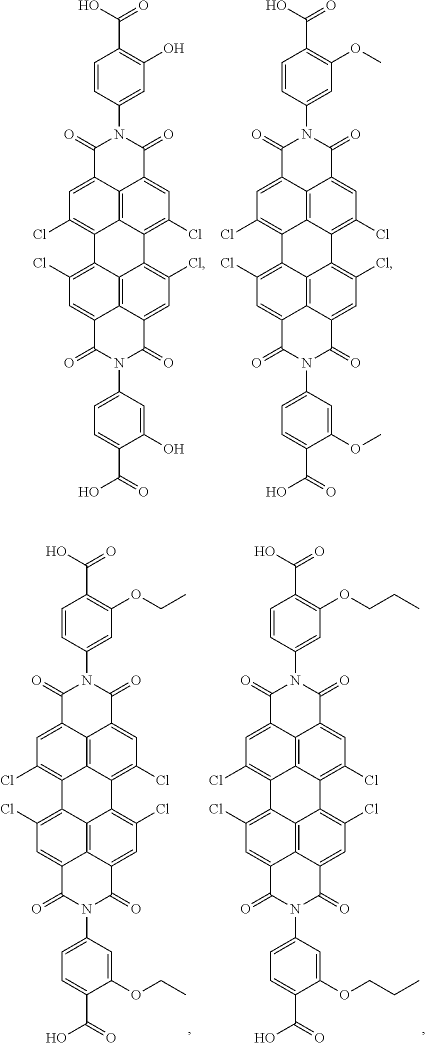

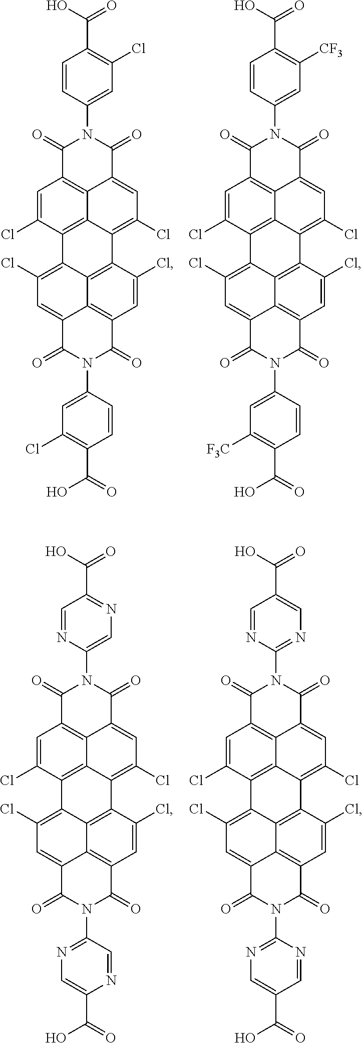

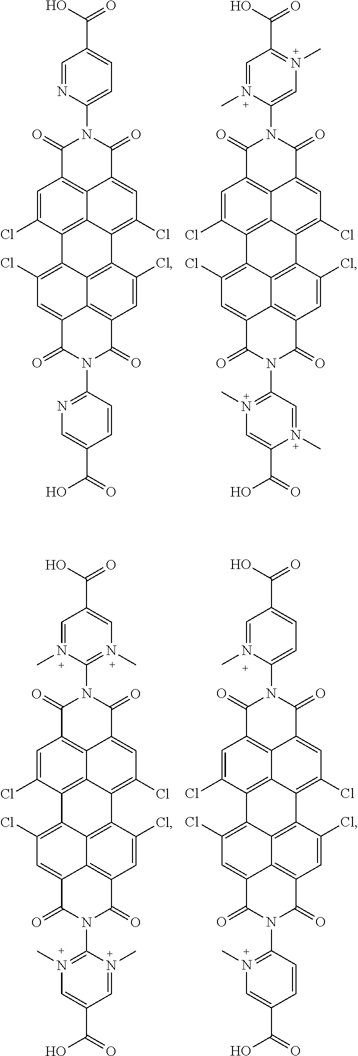

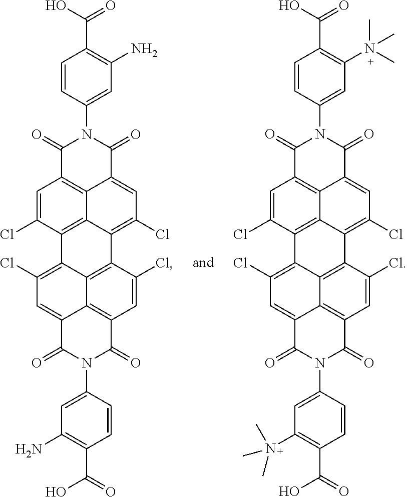

9. The composition of claim 1, wherein the secondary building units comprise one or more metal atoms and one or more carboxylates.

10. The composition of claim 9, wherein the metal atoms comprise zirconium(IV) or hafnium(IV).

11. The composition of claim 1, wherein the composition further comprises one or more modulators.

12. The composition of claim 11, wherein the modulators are trifluoroacetic acid.

13. A method for detecting or removing perfluoroalkyl or polyfluoroalkyl substances in a sample, the method comprising:
contacting the sample with a composition comprising at least one metal-organic framework (MOF), or salt thereof, having a plurality of metal-containing secondary building units (SBU) linked together by one or more bridging ligands,
wherein said bridging ligands, or salts thereof, have a formula (I): 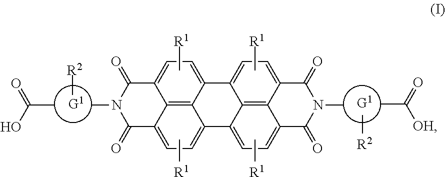
wherein
R¹, at each occurrence, is independently hydrogen, C₁₋₈alkyl, or halo;
R², at each occurrence, is independently hydrogen, C₁₋₈alkyl, —O(R^{1a}), or C₁₋₈alkyl-OH; and
R^{1a}, at each occurrence, is independently hydrogen or C₁₋₈alkyl; and
detecting a change in fluorescence intensity.

14. The method of claim 13, wherein the bridging ligands, or salts thereof, have a formula (Ia):

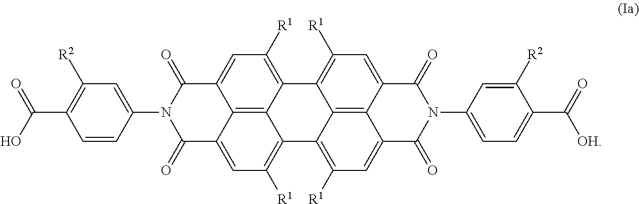

15. The method of claim 13, wherein R¹, at each occurrence, is halo.

16. The method of claim 13, wherein R¹, at each occurrence, is chloro.

17. The method of claim 13, wherein R² at each occurrence, is C₁₋₈alkyl.

18. The method of claim 13, wherein R², at each occurrence, is C₁alkyl.

19. The method of claim 13, wherein R², at each occurrence, is hydrogen.

20. The method of claim 13, wherein the bridging ligands, or salts thereof, are selected from:

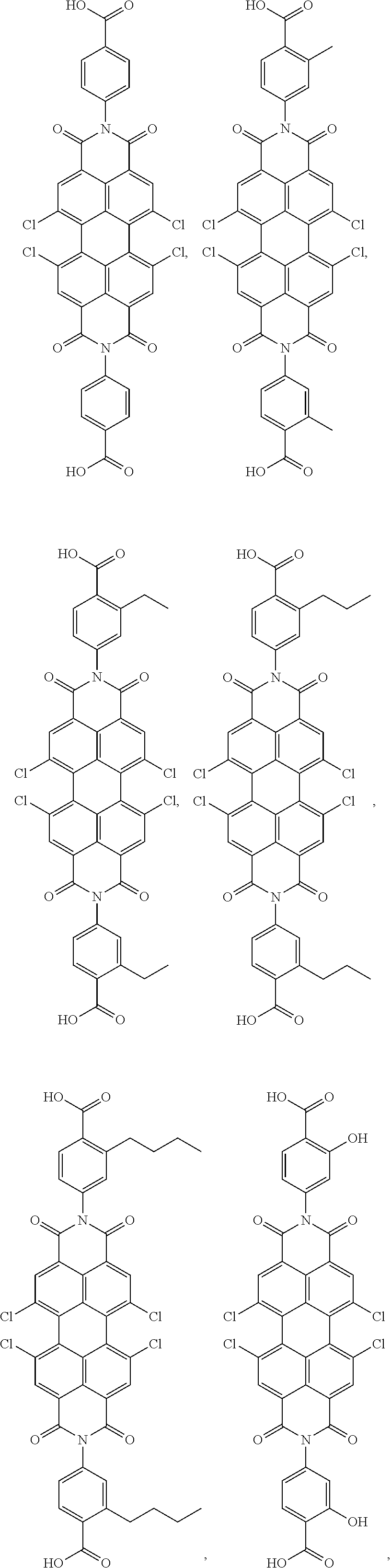

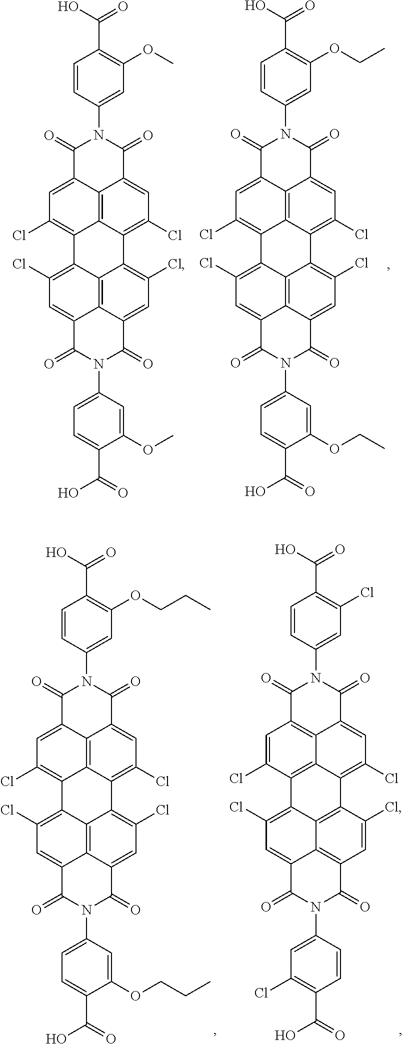

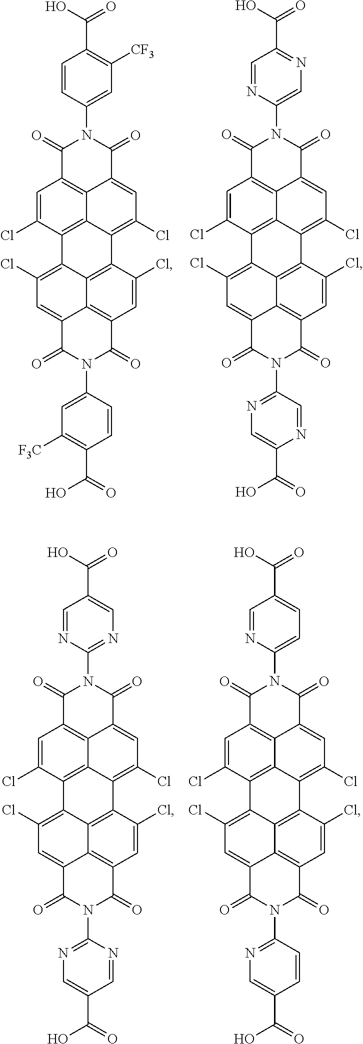

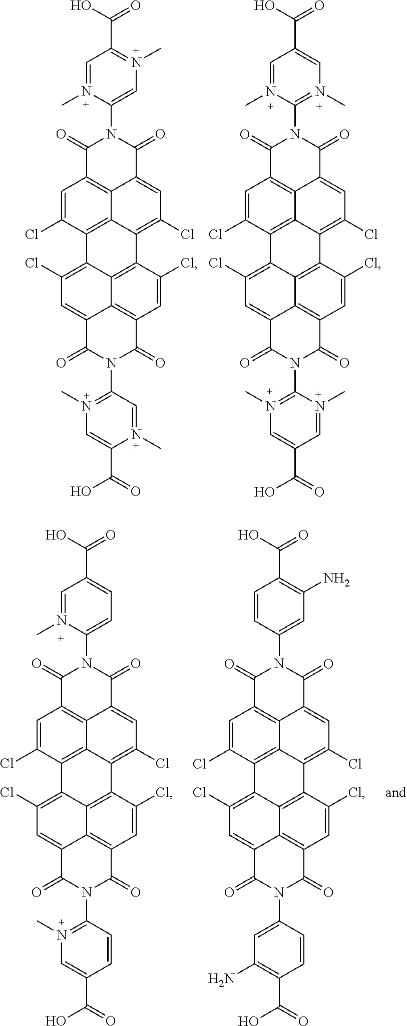

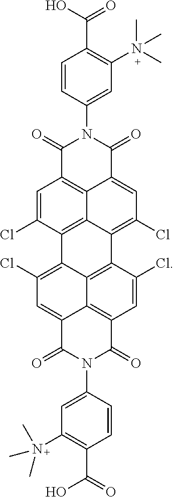

21. The method of claim 13, wherein the secondary building units comprise one or more metal atoms or one or more carboxylates.

22. The method of claim 21, wherein the metal atoms comprise zirconium(IV) or hafnium(IV).

23. The method off claim 13, wherein the composition further comprises one or more modulators.

24. The method of claim 23, wherein the one or more modulators are trifluoroacetic acid.

25. The method of claim 13, wherein the one or more perfluoroalkyl or polyfluoroalkyl substances is perfluooctanoic acid (PFOA).

26. The method of claim 13, wherein the composition is noncovalently adhered or covalently attached to a surface.

27. The method of claim 13, wherein the surface comprises cellulose, silica, a polymer, a copolymer, a resin, a filter, or a combination thereof.

28. The method of claim 13, wherein the composition is dissolved in a solvent.

29. The method of claim 13, wherein the change is an increase in fluorescence intensity.

30. The method of claim 13, wherein the change in fluorescence intensity indicate removal of the perfluoroalkyl or polyfluoroalkyl substances from the sample.

31. The method of claim 13, wherein the composition has a limit of detection of about 1-50 ppb.

32. A method for detecting or removing perfluoroalkyl or polyfluoroalkyl substances in a sample, the method comprising:
contacting the sample with a composition comprising at least one metal-organic framework (MOF), or salt thereof, having a plurality of metal-containing secondary building units (SBU) linked together by one or more bridging ligands,
wherein said bridging ligands, or salts thereof, have a formula (II): 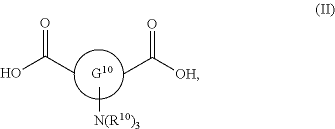
wherein
R¹⁰, at each occurrence, is independently hydrogen or C₁₋₈alkyl; and
G¹⁰ is independently a 6- to 12-membered aryl or a 5- to 12-membered heteroaryl.
detecting a change in fluorescence intensity.

33. The method of claim 32, wherein the bridging ligands have a formula (IIa):

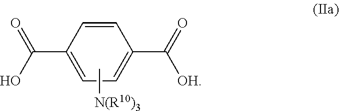

34. The method of claim 32, wherein R¹⁰ at each occurrence is C₁alkyl.

35. The method of claim 32, wherein the bridging ligands, or salts thereof, are selected from:

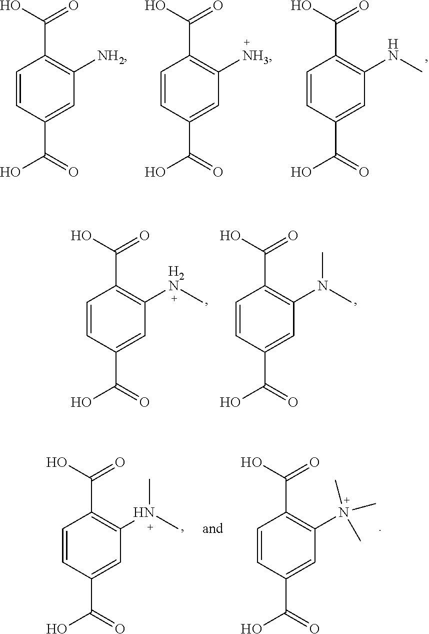

36. The method of claim 32, wherein the secondary building units comprise one or more metal atoms and one or more carboxylates.

37. The method of claim 36, wherein the metal atoms comprise zirconium(IV) or hafnium(IV).

38. The method of claim 32, wherein the one or more perfluoroalkyl or polyfluoroalkyl substances is perfluooctanoic acid (PFOA).

39. The method of claim 32, wherein the composition is noncovalently adhered or covalently attached to a surface.

40. The method of claim 32, wherein the surface comprises cellulose, silica, a polymer, a copolymer, a resin, a filter, or a combination thereof.

41. The method of claim 32, wherein the composition is dissolved in a solvent.

42. The method of claim 32, wherein the change is an increase in fluorescence intensity.

43. The method of claim 32, wherein the change in fluorescence intensity indicates removal of perfluoralkyl or polyfluoralkyl substances from the sample.

44. The method of claim 32, wherein the composition has a limit of detection of about 1-50 ppb.
